(19) United States
(12) Patent Application Publication
(10) Pub. No.: US 2021/0299265 A1
(43) Pub. Date: Sep. 30, 2021

(54) EFFICIENT PROCESS FOR PREPARING CELL-BINDING AGENT-CYTOTOXIC AGENT CONJUGATES

(71) Applicant: IMMUNOGEN, INC.

(72) Inventors: Katharine C. Lai, Littleton, MA (US); Robert W. Herbst, Braintree, MA (US); Scott Alan Hilderbrand, Swampscott, MA (US)

(73) Assignees: IMMUNOGEN, INC., Waltham, MA (US); IMMUNOGEN, INC., Waltham, MA (US)

(21) Appl. No.: 16/075,263

(22) PCT Filed: Feb. 3, 2017
(86) PCT No.: PCT/US2017/016344

§ 371 Date: Aug. 3, 2018

Related U.S. Application Data
(60) Provisional application No. 62/292,018, filed on Feb. 5, 2016.

(51) Int. Cl. A61K 47/68; A61K 31/5513; A61K 47/65

(52) U.S. Cl. CPC A61K 47/6803; A61K 47/65; A61K 31/5513

(57) ABSTRACT

The present invention provides a novel method for preparing a cell-binding agent cytotoxic agent conjugate. The method comprises the step of reacting a cell-binding agent with a cytotoxic agent or a cytotoxic agent-linker compound having a reactive group capable of forming a covalent bond with the cell-binding agent at a pH between 4 to 9 in the presence of a buffer solution with high ionic strength, wherein the cell-binding agent comprises a lysine ε-NH₂ group that forms a covalent bond with the cytotoxic agent or the cytotoxic agent-linker compound having an amine-reactive group. The cell-binding agent-cytotoxic agent conjugates prepared according to the methods described herein are also included in the present invention.

### RELATED APPLICATION

This application claims the benefit of the filing date under 35 U.S.C. § 119(e), of U.S. Provisional Application No. 62/292,018, filed on Feb. 5, 2016, the entire contents of each of which, including all drawings, formulae, specifications, and claims, are incorporated herein by reference.

### BACKGROUND OF THE INVENTION

Antibody-drug conjugates (ADCs) of indolinobenzodiazepine dimer compounds have been shown to have high potency and/or high therapeutic index (ratio of maximum tolerated dose to minimum effective dose) in vivo. Indolinobenzodiazepine dimer compounds are generally hydrophobic and may affect the stability of the antibody during the conjugation reaction. Under certain circumstances, the conjugation reaction has very low reaction yield, which is undesirable for large scale production of ADCs.

In view of the foregoing, there is an unmet need to develop efficient processes for preparing cell-binding agent-cytotoxic agent conjugates that are suitable for large scale productions.

### SUMMARY OF THE INVENTION

The present invention provides novel and efficient methods for preparing cell-binding agent-cytotoxic agent conjugates.

In one embodiment, the methods of the present invention comprises the step of reacting a cell-binding agent with a cytotoxic agent or a cytotoxic agent-linker compound having a reactive group (e.g., an amine-reactive group) capable of forming a covalent bond with the cell-binding agent at a pH between 4 to 9 in the presence of a buffer solution with high ionic strength.

In another embodiment, the method of the present invention comprises the step of reacting a cell-binding agent with a cytotoxic agent or a cytotoxic agent-linker compound having a reactive group (e.g., an amine-reactive group) capable of forming a covalent bond with the cell-binding agent in a buffer solution having a pH of 7.3 to 8.4.

In yet another embodiment, the methods of the present invention comprises the step of reacting a cell-binding agent with a cytotoxic agent or a cytotoxic agent-linker compound having a reactive group (e.g., an amine-reactive group) capable of forming a covalent bond with the cell-binding agent at a pH between 4 to 9 in the presence of a high concentration buffer solution.

It has been surprisingly found that when the conjugation reaction of an indolinobenzodiazepine dimer compound and an antibody is carried out in a buffer solution with high ionic strength at a pH between 7.3 and 8.4, the conjugation reaction is significantly more efficient compared to when the conjugation reaction is carried out with a buffer solution having low ionic strength at a higher pH. The methods of the present invention provide cell-binding agent-cytotoxic agent conjugates with high purity and/or stability.

The present invention is also directed to the cell-binding agent cytotoxic agent conjugates prepared using the methods described herein.

### DETAILED DESCRIPTION OF THE INVENTION

The present invention provides novel methods for preparing a cell-binding agent-cytotoxic agent conjugate.

In a first embodiment, the method of the present invention comprises the step of reacting a cell-binding agent with a cytotoxic agent or a cytotoxic agent-linker compound having a reactive group (e.g., an amine-reactive group) capable of forming a covalent bond with the cell-binding agent at a pH between 4 to 9 in the presence of a buffer solution with high ionic strength.

As used herein, “ionic strength” of a solution is the concentration of ions in the solution. It is a function of the concentration of all ions present in the solution. The ionic strength (I) can be calculated using the following equation:

I=½ΣCᵢzᵢ²

Cᵢ is the molar concentration of ion i present in the solution, zᵢ is its charge number, and the sum is taken over all ions in the solution. When the cation and anion of the solution carry +1 and −1 charge respectively, the ionic strength is equal to the concentration of the solution.

In one embodiment, the ionic strength of the buffer solution is between 20 mM and 500 mM, preferably between 20 mM and 200 mM, between 25 mM and 150 mM, between 50 mM and 150 mM, between 50 mM and 100 mM, or between 100 mM and 200 mM. In another embodiment, the ionic strength of the buffer solution is between 60 mM and 90 mM, or between 70 mM and 80 mM. In yet another embodiment, the ionic strength of the buffer solution is 75 mM. In another embodiment, the ionic strength of the buffer solution is 100 mM to 160 mM or between 120 mM and 140 mM. In yet another embodiment, the ionic strength of the buffer solution is 130 mM.

In another embodiment, the pH of the buffer solution is between 7.1 and 8.7, preferably between 7.3 and 8.7, between 7.1 and 8.5, between 7.3 and 8.4, between 7.6 and 8.4, between 7.7 and 8.3, between 7.8 and 8.2. In one embodiment, the pH of the buffer solution is between 7.9 and 8.1. In another embodiment, the pH of the buffer solution is at 8.0. In one embodiment, the pH of the buffer solution is between 8.5 and 8.9. In another embodiment, the pH of the buffer solution is between 8.6 and 8.8. In yet another embodiment, the pH of the buffer solution is 8.7.

In a second embodiment, the method of the present invention comprises the step of reacting a cell-binding agent with a cytotoxic agent or a cytotoxic agent-linker compound having a reactive group (e.g., an amine-reactive group) capable of forming a covalent bond with the cell-binding agent in a buffer solution having a pH of 7.3 to 9.0.

In one embodiment, the pH of the buffer solution is between 7.3 and 8.4, between 7.6 and 8.4, between 7.7 and 8.3, or between 7.8 and 8.2. In another embodiment, the pH of the buffer solution is between 7.9 and 8.1. In another embodiment, the pH of the buffer solution is at 8.0. In one embodiment, the pH of the buffer solution is between between 8.5 and 8.9. In another embodiment, the pH of the buffer solution is between 8.6 and 8.8. In yet another embodiment, the pH of the buffer solution is 8.7.

In a 1^{st} specific embodiment, for the method described in the first or the second embodiment, the buffer solution has an ionic strength between 20 mM and 200 mM and a pH between 7.1 and 8.5.

In a 2^{nd} specific embodiment, for the method described in the first or the second embodiment, the buffer solution has an ionic strength between 50 mM and 150 mM and a pH between 7.6 and 8.4.

In a 3^{rd} specific embodiment, for the method described in the first or the second embodiment, the buffer solution has an ionic strength between 50 mM and 100 mM and a pH between 7.7 and 8.3.

In a 4^{th} specific embodiment, for the method described in the first or the second embodiment, the buffer solution has an ionic strength between 60 mM and 90 mM and a pH between 7.8 and 8.2.

In a 5^{th} specific embodiment, for the method described in the first or the second embodiment, the buffer solution has an ionic strength between 70 mM and 80 mM and a pH between 7.9 and 8.1.

In a 6^{th} specific embodiment, for the method described in the first or the second embodiment, the buffer solution has an ionic strength of 75 mM and a pH of 8.0.

In a 7^{th} specific embodiment, for the method described in the first or the second embodiment, the buffer solution has an ionic strength between 50 mM and 200 mM and a pH between 7.8 and 8.9.

In a 8^{th} specific embodiment, for the method described in the first or the second embodiment, the buffer solution has an ionic strength between 110 mM and 150 mM and a pH between 8.5 and 8.9.

In a 9^{th} specific embodiment, for the method described in the first or the second embodiment, the buffer solution has an ionic strength between 120 mM and 140 mM and a pH between 8.6 and 8.8.

In a 10^{th} specific embodiment, for the method described in the first or the second embodiment, the buffer solution has an ionic strength of 130 mM and a pH of 8.7.

Any suitable buffer solution known in the art can be used in the methods of the present invention. Suitable buffer solutions include, for example, but are not limited to, a citrate buffer, an acetate buffer, a succinate buffer, and a phosphate buffer.

In a 11^{th} specific embodiment, for the method described in the first or the second embodiment, or the 1^{st}, 2^{nd}, 3^{rd}, 4^{th}, 5^{th}, 6^{th}, 7^{th}, 8^{th}, 9^{th}, or 10^{th} specific embodiment, the buffer solution is selected from the group consisting of MES ((2-(N-morpholino)ethanesulfonic acid)) buffer, bis-tris methane (2-[Bis(2-hydroxyethyl)amino]-2-(hydroxymethyl)propane-1,3-diol) buffer, ADA (N-(2-Acetamido)iminodiacetic acid) buffer, ACES (N-2-aminoethanesulfonic acid) buffer, PIPES (piperazine-N,N′-bis(2-ethanesulfonic acid)), MOPSO (β-Hydroxy-4-morpholinepropanesulfonic acid) buffer, bis-tris propane (1,3-bis(tris(hydroxymethyl)methylamino)propane) buffer, BES (N,N-bis(2-hydroxyethyl)-2-aminoethanesulfonic acid), TES (N-[tris(hydroxymethyl)methyl]-2-aminoethanesulfonic acid) buffer, HEPES (4-(2-hydroxyethyl)piperazine-1-ethanesulfonic acid) buffer, DIPSO, (3-(N,N-Bis[2-hydroxyethyl]amino)-2-hydroxypropanesulfonic acid or N,N-Bis(2-hydroxyethyl)-3-amino-2-hydroxypropanesulfonic acid), MOBS (4-(N-morpholino)butanesulfonic acid) buffer, TAPSO (3-[[1,3-dihydroxy-2-(hydroxymethyl)propan-2-yl]amino]-2-hydroxypropane-1-sulfonic acid) buffer, trizma (Tris or 2-Amino-2-(hydroxymethyl)-1,3-propanediol) buffer, HEPPSO (N-(2-hydroxyethyl)piperazine-N′-(2-hydroxypropanesulfonic acid)) buffer, POPSO (piperazine-1,4-bis-(2-hydroxy-propane-sulfonic acid) dehydrate) buffer, EPPS (4-(2-hydroxyethyl)piperazine-1-propanesulfonic acid) buffer, tricine (N-(2-Hydroxy-1,1-bis(hydroxymethyl)ethyl)glycine) bufer, gly-gly, bicine (2-(Bis(2-hydroxyethyl)amino)acetic acid) buffer, HEPBS (N-(2-Hydroxyethyl)piperazine-N′-(4-butanesulfonic acid)) buffer, TAPS (3-[[1,3-dihydroxy-2-(hydroxymethyl)propan-2-yl]amino]propane-1-sulfonic acid) buffer, AMPD (2-amino-2-methyl-1,3-propanediol) buffer, TABS (N-tris(Hydroxymethyl)methyl-4-aminobutanesulfonic acid) buffer, AMPSO (N-(1,1-Dimethyl-2-hydroxyethyl)-3-amino-2-hydroxypropanesulfonic acid) buffer and a combination thereof.

In one embodiment, the buffer is selected from the group consisting of HEPPSO (N-(2-hydroxyethyl)piperazine-N′-(2-hydroxypropanesulfonic acid)) buffer, POPSO (piperazine-1,4-bis-(2-hydroxy-propane-sulfonic acid) dehydrate) buffer, HEPES (4-(2-hydroxyethyl)piperazine-1-ethanesulfonic acid) buffer, EPPS (4-(2-hydroxyethyl)piperazine-1-propanesulfonic acid) buffer, TES (N-[tris(hydroxymethyl)methyl]-2-aminoethanesulfonic acid) buffer, MES (2-(N-morpholino)ethanesulfonic acid) buffer and a combination thereof.

In a 12^{th} specific embodiment, for the method described in the first or the second embodiment, or the 1^{st}, 2^{nd}, 3^{rd}, 4^{th}, 5^{th}, 6^{th}, 7^{th}, 8^{th}, 9^{th} or 10^{th} specific embodiment, the buffer solution is a EPPS buffer. In a preferred embodiment, the buffer solution is a 75 mM EPPS buffer.

In a 13^{th} specific embodiment, for the method described in the first or the second embodiment, the buffer solution is 50 mM to 200 mM EPPS buffer having a pH between 7.8 and 8.9.

In a 14^{th} specific embodiment, for the method described in the first or the second embodiment, the buffer solution is 60 mM to 90 mM EPPS buffer having a pH between 7.8 and 8.2.

In a 15^{th} specific embodiment, for the method described in the first or the second embodiment, the buffer solution is 70 mM to 80 mM EPPS buffer having a pH between 7.9 and 8.1.

In a 16^{th} specific embodiment, for the method described in the first or the second embodiment, the buffer solution is 75 mM EPPS buffer having a pH of 8.0.

In a 17^{th} specific embodiment, for the method described in the first or the second embodiment, the buffer solution is 110 mM to 150 mM EPPS buffer having a pH between 8.5 and 8.9.

In a 18^{th} specific embodiment, for the method described in the first or the second embodiment, the buffer solution is 120 mM to 140 mM EPPS buffer having a pH between 8.6 and 8.8.

In a 19^{th} specific embodiment, for the method described in the first or the second embodiment, the buffer solution is 130 mM EPPS buffer having a pH of 8.7.

In a third embodiment, the method of the present invention comprises the step of reacting a cell-binding agent with a cytotoxic agent or a cytotoxic agent-linker compound having a reactive group (e.g., an amine-reactive group) capable of forming a covalent bond with the cell-binding agent at a pH between 4 to 9 in the presence of a high concentration buffer.

In one embodiment, the concentration of the buffer is between 20 mM and 750 mM. In another embodiment, the concentration of the buffer is between 20 mM and 500 mM, between 20 mM and 200 mM, between 25 mM and 150 mM, between 50 mM and 150 mM, between 50 mM and 100 mM, between 100 mM and 200 mM, or between 100 mM and 150 mM.

In one embodiment, the pH of the buffer solution is between 7.3 and 8.9, between 7.3 and 8.4, between 7.6 and 8.4, between 7.7 and 8.3, or between 7.8 and 8.2. In another embodiment, the pH of the buffer solution is between 7.9 and 8.1. In another embodiment, the pH of the buffer solution is at 8.0. In one embodiment, the pH of the buffer solution is between between 8.5 and 8.9. In another embodiment, the pH of the buffer solution is between 8.6 and 8.8. In yet another embodiment, the pH of the buffer solution is 8.7.

In a 20^{th} specific embodiment, for the method described in the third embodiment, the buffer solution has a concentration between 20 mM and 200 mM and a pH between 7.1 and 8.5.

In a 21^{st} specific embodiment, for the method described in the third embodiment, the buffer solution has a concentration between 50 mM and 150 mM and a pH between 7.6 and 8.4.

In a 22^{nd} specific embodiment, for the method described in the first or the second embodiment, the buffer solution has a concentration between 50 mM and 100 mM and a pH between 7.7 and 8.3.

In a 23^{rd} specific embodiment, for the method described in the third embodiment, the buffer solution has a concentration between 60 mM and 90 mM and a pH between 7.8 and 8.2.

In a 24^{th} specific embodiment, for the method described in the third embodiment, the buffer solution has a concentration between 70 mM and 80 mM and a pH between 7.9 and 8.1.

In a 25^{th} specific embodiment, for the method described in the third embodiment, the buffer solution has a concentration of 75 mM and a pH of 8.0.

In a 26^{th} specific embodiment, for the method described in the third embodiment, the buffer solution has a concentration between 50 mM and 200 mM and a pH between 7.8 and 8.9.

In a 27^{th} specific embodiment, for the method described in the third embodiment, the buffer solution has a concentration between 110 mM and 150 mM and a pH between 8.5 and 8.9.

In a 28^{th} specific embodiment, for the method described in the third embodiment, the buffer solution has a concentration between 120 mM and 140 mM and a pH between 8.6 and 8.8.

In a 29^{th} specific embodiment, for the method described in the third embodiment, the buffer solution has a concentration of 130 mM and a pH of 8.7.

In one embodiment, the buffer solution used in the methods of the present invention may further comprise an inert salt to maintain the ionic strength of the buffer. In one embodiment, the buffer solution further comprises sodium chloride.

In one embodiment, for the methods of the present invention described above, the reaction between the cell-binding agent and the cytotoxic agent or the cytotoxic agent-linker compound is carried out in the presence of small amount of organic solvent. More specifically, the organic solvent is dimethylacetamide (DMA). The organic solvent (e.g., DMA) can be present in the amount of 1%-20%, 1-15%, 2-15%, 5-15%, 8-12%, or 10-20% by volume of the total volume of the buffer solution and the organic solvent. In one embodiment, the organic solvent (e.g., DMA) is present in the amount of 10% by volume of the total volume of the buffer solution and the organic solvent. In another embodiment, the organic solvent (e.g., DMA) is present in the amount of 15% by volume of the total volume of the buffer solution and the organic solvent.

In one embodiment, for the methods of the present invention described above, the reaction is allowed to proceed for 2 minutes to 1 week, 1 hour to 48 hours, 1 hour to 36 hours, 1 hour to 24 hours, 1 hour to 12 hours, 1 hours to 8 hours, 5 hours to 15 hours, 6 hours to 14 hours, 4 hours to 8 hours, 5 hours to 7 hours, 1 hours to 5 hours, 1 hours to 4 hours, 1 hours to 2 hours, 30 minutes to 2 hour, 5 minutes to 30 minutes, or 2 hours to 5 hours. In one embodiment, the reaction is allowed to proceed for 2 hours to 6 hours or 3 hours to 5 hours. In one embodiment, the reaction is allowed to proceed for 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, etc. In another embodiment, the reaction is allowed to proceed for 4 hours.

The reaction between the cell-binding agent and the cytotoxic agent or the cytotoxic agent-linker compound can be carried out at any suitable temperature. In one embodiment, the reaction can be carried out at a temperature from 10° C. to 50° C., from 10° C. to 40° C., or from 10° C. to 30° C. In another embodiment, the reaction can be carried out at a temperature from 15° C. to 30° C., 20° C. to 30° C., 15° C. to 25° C., from 16° C. to 24° C., from 17° C. to 23° C., from 18° C. to 22° C. or from 19° C. to 21° C. In yet another embodiment, the reaction can be carried out at 15° C., 16° C., 17° C., 18° C., 19° C., 20° C., 21° C., 22° C., 23° C., 24° C. or 25° C.

The conjugate formed from the conjugation reaction between the cell-binding agent and the cytotoxic agent or the cytotoxic agent-linker compound may have a tendency to form high molecular weight species upon storage or during the time between the completion of the conjugation reaction and the purification step. To mitigate the formation of the high molecular weight species, a quenching solution may be added after the conjugation reaction to stabilize the conjugate.

In a 30^{th} specific embodiment, the method described in the first, second or third embodiment above (e.g., in the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, or 29^{th} specific embodiment) further comprises the step of adding a quenching solution with high ionic strength after the reaction of the cytotoxic agent or the cytotoxic agent-linker compound with the cell-binding agent.

Also provided in the 30^{th} specific embodiment, the method further comprises the step of adding a quenching solution comprising a high concentration buffer after the reaction of the cytotoxic agent or the cytotoxic agent-linker compound with the cell-binding agent.

In one embodiment, the quenching solution has an ionic strength between 200 mM and 3000 mM, between 200 mM and 2000 nM, between 200 mM and 1000 mM, 500 mM and 1000 mM, between 550 mM and 1000 mM, or between 600 mM and 1000 mM. In another embodiment, the quenching solution has an ionic strength between 700 mM and 1000 mM. In another embodiment, the quenching solution has an ionic strength of 900 mM.

In another embodiment, the quenching solution comprises a buffer with a concentration between 200 mM and 3000 mM, between 200 mM and 2000 mM, between 200 mM and 1000 mM, 500 mM and 1000 mM, between 550 mM and 1000 mM, or between 600 mM and 1000 mM. In another embodiment, the quenching solution has a buffer with a concentration between 700 mM and 1000 mM. In another embodiment, the quenching solution has a buffer with a concentration of 750 mM.

In another embodiment, the quenching solution was mixed with the reaction mixture after the reaction of the cytotoxic agent or the cytotoxic agent-linker compound with the cell-binding agent and subsequent to the mixing, the final concentration for the buffer is between 150 mM and 750 mM, between 150 mM and 600 mM, between 200 mM and 500 nM, between 200 mM and 400 nM, between 250 mM and 350 mM.

In some embodiments, the buffer in the quenching solution is the same as the buffer used in the conjugation reaction of the cytotoxic agent or the cytotoxic agent-linker compound with the cell-binding agent.

The quenching solution described herein can comprise a buffer, a salt or a combination therefore. Any suitable buffer or salt can be used. Exemplary buffers include, but are not limited to, MES ((2-(N-morpholino)ethanesulfonic acid)) buffer, bis-tris methane (2-[Bis(2-hydroxyethyl)amino]-2-(hydroxymethyl)propane-1,3-diol) buffer, ADA (N-(2-Acetamido)iminodiacetic acid) buffer, ACES (N-2-aminoethanesulfonic acid) buffer, PIPES (piperazine-N,N′-bis(2-ethanesulfonic acid)), MOPSO (β-Hydroxy-4-morpholinepropanesulfonic acid) buffer, bis-tris propane (1,3-bis(tris(hydroxymethyl)methylamino)propane) buffer, BES (N,N-bis(2-hydroxyethyl)-2-aminoethanesulfonic acid), TES (N-[tris(hydroxymethyl)methyl]-2-aminoethanesulfonic acid) buffer, HEPES (4-(2-hydroxyethyl)piperazine-1-ethanesulfonic acid) buffer, DIPSO, (3-(N,N-Bis[2-hydroxyethyl]amino)-2-hydroxypropanesulfonic acid or N,N-Bis(2-hydroxyethyl)-3-amino-2-hydroxypropanesulfonic acid), MOBS (4-(N-morpholino)butanesulfonic acid) buffer, TAPSO (3-[[1,3-dihydroxy-2-(hydroxymethyl)propan-2-yl]amino]-2-hydroxypropane-1-sulfonic acid) buffer, trizma (Tris or 2-Amino-2-(hydroxymethyl)-1,3-propanediol) buffer, HEPPSO (N-(2-hydroxyethyl)piperazine-N′-(2-hydroxypropanesulfonic acid)) buffer, POPSO (piperazine-1,4-bis-(2-hydroxy-propane-sulfonic acid) dehydrate) buffer, EPPS (4-(2-hydroxyethyl)piperazine-1-propanesulfonic acid) buffer, tricine (N-(2-Hydroxy-1,1-bis(hydroxymethyl)ethyl)glycine) bufer, gly-gly, bicine (2-(Bis(2-hydroxyethyl)amino)acetic acid) buffer, HEPBS (N-(2-Hydroxyethyl)piperazine-N′-(4-butanesulfonic acid)) buffer, TAPS (3-[[1,3-dihydroxy-2-(hydroxymethyl)propan-2-yl]amino]propane-1-sulfonic acid) buffer, AMPD (2-amino-2-methyl-1,3-propanediol) buffer, TABS (N-tris(Hydroxymethyl)methyl-4-aminobutanesulfonic acid) buffer, AMPSO (N-(1,1-Dimethyl-2-hydroxyethyl)-3-amino-2-hydroxypropanesulfonic acid) buffer and a combination thereof. In one embodiment, the buffer is selected from the group consisting of HEPPSO (N-(2-hydroxyethyl)piperazine-N′-(2-hydroxypropanesulfonic acid)) buffer, POPSO (piperazine-1,4-bis-(2-hydroxy-propane-sulfonic acid) dehydrate) buffer, HEPES (4-(2-hydroxyethyl)piperazine-1-ethanesulfonic acid) buffer, EPPS (4-(2-hydroxyethyl)piperazine-1-propanesulfonic acid) buffer, TES (N-[tris(hydroxymethyl)methyl]-2-aminoethanesulfonic acid) buffer, MES (2-(N-morpholino)ethanesulfonic acid) buffer and a combination thereof. Exemplary salts include, but are not limited, NaCl, KCl, and histidine hydrochloride. In one embodiment, the quenching solution comprises EPPS. In another embodiment, the quenching solution comprises EPPS and histidine hydrochloride.

In one embodiment, the quenching solution has a pH between 5 and 9, between 5 and 7 or between 5 and 6. In another embodiment, the quenching solution has a pH of 5.5.

In one embodiment, the quenching solution before mixing with the reaction mixture comprises 750 mM EPPS and 150 mM of histidine hydrochloride.

In one embodiment, the quenching solution comprises EPPS and histidine hydrochloride and subsequent to mixing the quenching solution with the reaction mixture, the resulting mixture comprises 200 mM to 400 mM EPPS and 40 to 60 mM histidine hydrochloride. In one embodiment, the resulting mixture comprises 250 mM to 350 mM EPPS and 40 to 60 mM histidine hydrochloride. In yet another embodiment, the resulting mixture comprises 300 mM to 350 mM EPPS and 45 mM to 55 mM histidine hydrochloride.

In one embodiment, the cell-binding agent-cytotoxic agent conjugate prepared according to the methods of the present invention is subjected to a purification step. In this regard, the cell-binding agent-cytotoxic agent conjugate can be purified from the other components of the mixture (e.g., free cytotoxic agent or cytotoxic agent-linker compound and reaction by-products) using tangential flow filtration (TFF), which is a membrane-based tangential flow filtration process, non-adsorptive chromatography, adsorptive chromatography, adsorptive filtration, selective precipitation, or any other suitable purification process, as well as combinations thereof.

In one embodiment of the invention, the cell-binding agent-cytotoxic agent conjugate is purified using a single purification step (e.g., TFF). Preferably, the conjugate is purified and exchanged into the appropriate formulation using a single purification step (e.g., TFF). In another embodiment of the invention, the cell-binding agent cytotoxic agent conjugate is purified using two sequential purification steps. For example, the conjugate can be first purified by selective precipitation, adsorptive filtration, absorptive chromatography or non-absorptive chromatography, followed by purification with TFF. One of ordinary skill in the art will appreciate that purification of the cell-binding agent-cytotoxic agent conjugate enables the isolation of a stable conjugate comprising the cell-binding agent chemically coupled to the cytotoxic agent.

Any suitable TFF systems may be utilized for purification, including a Pellicon type system (Millipore, Billerica, Mass.), a Sartocon Cassette system (Sartorius AG, Edgewood, N.Y.), TangenX cassette (TangenX Technology Corporation, Shrewsbury, Mass.) and a Centrasette type system (Pall Corp., East Hills, N.Y.)

Any suitable adsorptive chromatography resin may be utilized for purification, wherein the resin may retain either the cell-binding agent-cytotoxic agent conjugate and permit elution of the impurities or retain the impurities and permit elution of the cell-binding agent-cytotoxic agent conjugate. Preferred adsorptive chromatography resins include hydroxyapatite chromatography, hydrophobic charge induction chromatography (HCIC), hydrophobic interaction chromatography (HIC), ion exchange chromatography, mixed mode ion exchange chromatography, immobilized metal affinity chromatography (IMAC), dye ligand chromatography, affinity chromatography, reversed phase chromatography, and combinations thereof. Examples of suitable hydroxyapatite resins include ceramic hydroxyapatite (CHT Type I and Type II, Bio-Rad Laboratories, Hercules, Calif.), HA Ultrogel hydroxyapatite (Pall Corp., East Hills, N.Y.), and ceramic fluoroapatite (CFT Type I and Type II, Bio-Rad Laboratories, Hercules, Calif.). An example of a suitable HCIC resin is MEP Hypercel resin (Pall Corp., East Hills, N.Y.). Examples of suitable HIC resins include Butyl-Sepharose, Hexyl-Sepaharose, Phenyl-Sepharose, and Octyl Sepharose resins (all from GE Healthcare, Piscataway, N.J.), as well as Macro-prep Methyl and Macro-Prep t-Butyl resins (Biorad Laboratories, Hercules, Calif.). Examples of suitable ion exchange resins include SP-Sepharose, CM-Sepharose, and Q-Sepharose resins (all from GE Healthcare, Piscataway, N.J.), and Unosphere S resin (Bio-Rad Laboratories, Hercules, Calif.). Examples of suitable mixed mode ion exchangers include Bakerbond ABx resin (JT Baker, Phillipsburg N.J.) Examples of suitable IMAC resins include Chelating Sepharose resin (GE Healthcare, Piscataway, N.J.) and Profinity IMAC resin (Bio-Rad Laboratories, Hercules, Calif.). Examples of suitable dye ligand resins include Blue Sepharose resin (GE Healthcare, Piscataway, N.J.) and Affi-gel Blue resin (Bio-Rad Laboratories, Hercules, Calif.). Examples of suitable affinity resins include Protein A Sepharose resin (e.g., MabSelect, GE Healthcare, Piscataway, N.J.), where the cell-binding agent is an antibody, and lectin affinity resins, e.g. Lentil Lectin Sepharose resin (GE Healthcare, Piscataway, N.J.), where the cell-binding agent bears appropriate lectin binding sites. Alternatively an antibody specific to the cell-binding agent may be used. Such an antibody can be immobilized to, for instance, Sepharose 4 Fast Flow resin (GE Healthcare, Piscataway, N.J.). Examples of suitable reversed phase resins include C4, C8, and C18 resins (Grace Vydac, Hesperia, Calif.).

Any suitable non-adsorptive chromatography resin may be utilized for purification. Examples of suitable non-adsorptive chromatography resins include, but are not limited to, SEPHADEX™ G-25, G-50, G-100, SEPHACRYL™ resins (e.g., S-200 and S-300), SUPERDEX™ resins (e.g., SUPERDEX™ 75 and SUPERDEX™ 200), BIO-GEL® resins (e.g., P-6, P-10, P-30, P-60, and P-100), and others known to those of ordinary skill in the art.

The conjugate prepared by the methods described herein can be formulated in a suitable formulation buffer.

The cell-binding agent-cytotoxic agent conjugates prepared by the processes of the present invention have substantially high purity and stability. In one aspect of the invention, a cell-binding agent-cytotoxic agent conjugate of substantially high purity has one or more of the following features: (a) less than 25%, less than 20%, less than 15% (e.g., less than or equal to 15%, 14%, 13%, 12%, 11%, 10%, 9%, 8%, 7%, 6%, 5%, 4%, 3%, 2% or 1%) of antibody fragmentation, (b) greater than 90% (e.g., greater than or equal to 91%, 92%, 93%, 94%, 95%, 96%, 97%, 98%, 99%, or 100%), preferably greater than 95%, of conjugate species are monomeric, (c) unconjugated linker level in the conjugate preparation is less than about 10% (e.g., less than or equal to about 9%, 8%, 7%, 6%, 5%, 4%, 3%, 2%, 1%, or 0%) (relative to total linker), (d) less than 10% of conjugate species are crosslinked (e.g., less than or equal to about 9%, 8%, 7%, 6%, 5%, 4%, 3%, 2%, 1%, or 0%), (e) the level of free cytotoxic agent or cytotoxic agent-linker compound in the conjugate preparation is less than about 2% (e.g., less than or equal to about 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, or 0%) (mol/mol relative to total cytotoxic agent), (f) less than about 10%, less than about 5% (e.g., less than or equal to about 4%, 3%, 2%, 1% or 0%) of high molecular weight species; and/or (g) no substantial increase in the level of free cytotoxic agent upon storage (e.g., after about 1 week, about 2 weeks, about 3 weeks, about 1 month, about 2 months, about 3 months, about 4 months, about 5 months, about 6 months, about 1 year, about 2 years, about 3 years, about 4 years, or about 5 years). “Substantial increase” in the level of free cytotoxic agent means that after certain storage time (e.g., about 1 week, about 2 weeks, about 3 weeks, about 1 month, about 2 months, about 3 months, about 4 months, about 5 months, about 6 months, about 1 year, about 2 years, about 3 years, about 4 years, or about 5 years), the increase in the level of free cytotoxic agent is less than about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.2%, about 2.5%, about 2.7%, about 3.0%, about 3.2%, about 3.5%, about 3.7%, or about 4.0%.

As used herein, the term “unconjugated linker” refers to the cell-binding agent that is covalently linked with the bifunctional crosslinking reagent, wherein the cell-binding agent is not covalently coupled to the cytotoxic agent through the linker of the bifunctional crosslinking reagent (i.e., the “unconjugated linker” can be represented by CBA-L, wherein CBA represents the cell-binding agent and L represents the bifunctional crosslinking reagent. In contrast, the cell-binding agent cytotoxic agent conjugate can be represented by CBA-L-D, wherein D represents the cytotoxic agent).

As used herein, the term “high molecular weight species” or “HMW” refers to antibody-containing or conjugate-containing species that are high in molecular weight. The high molecular weight species can be dimer, trimer, other higher order oligomers formed by aggregation of the antibody or conjugate or the combination thereof. The high molecular weight species can be identified and its amount determined by SEC-HPLC.

In one embodiment, the average molar ratio of the cytotoxic agent to the cell-binding agent (i.e., DAR) in the cell-binding agent-cytotoxic agent conjugate is from 1 to 15, 1 to 10, 1 to 9, 1 to 8, 1 to 7, 1 to 6, 1 to 7, 1 to 6, 1 to 5, 1 to 4, 1 to 3, 1 to 2, from 1.5 to 5, from 2 to 7, or from 3 to 5. In another embodiment the DAR is from 1.5 to 3.5, from 2 to 3, from 2.1 to 2.9, from 2.2 to 2.8, from 2.3 to 2.7, or from 2.4 to 2.6. In another embodiment, the DAR for the conjugates prepared by the methods of the present invention is 2.0, 2.1, 2.2, 2.3, 2.4, 2.5, 2.5, 2.7, 2.8, 2.9 or 3.0. In one embodiment, the DAR is 2.5. In another embodiment, the DAR is 2.7.

The DAR value can be determined by any methods known in the art. In one embodiment, the DAR value can be determined by UV/Vis spectroscopy using the absorbance values at wavelengths for antibodies and cytotoxic agent, respectively. Alternatively, the DAR value can be determined by mass spectrometry and/or HPLC.

### Cell-Binding Agent

For use in the methods of the present invention, the cell-binding agent can be any suitable agent that binds to a cell, typically and preferably an animal cell (e.g., a human cell). The cell-binding agent preferably is a peptide or a polypeptide. Suitable cell-binding agents include, for example, antibodies (e.g., monoclonal antibodies and fragments thereof), interferons (e.g. alpha., beta., gamma.), lymphokines (e.g., IL-2, IL-3, IL-4, IL-6), hormones (e.g., insulin, TRH (thyrotropin releasing hormone), MSH (melanocyte-stimulating hormone), steroid hormones, such as androgens and estrogens), growth factors and colony-stimulating factors such as EGF, TGF-alpha, FGF, VEGF, G-CSF, M-CSF and GM-CSF (Burgess, Immunology Today 5:155-158 (1984)), nutrient-transport molecules (e.g., transferrin), vitamins (e.g., folate) and any other agent or molecule that specifically binds a target molecule on the surface of a cell.

Where the cell-binding agent is an antibody, it binds to an antigen that is a polypeptide or a glycotope and may be a transmembrane molecule (e.g., receptor) or a ligand such as a growth factor. Exemplary antigens include molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor vmc, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin, such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-beta.; platelet-derived growth factor (PDGF); fibroblast growth factor such as aFGF and bFGF; epidermal growth factor (EGF); transforming growth factor (TGF) such as TGF-alpha and TGF-beta, including TGF-.beta.1, TGF-.beta.2, TGF-.beta.3, TGF-.beta.4, or TGF-.beta.5; insulin-like growth factor-I and -II (IGF-I and IGF-II); des(1-3)-IGF-I (brain IGF-I); insulin-like growth factor binding proteins; EpCAM; GD3; FLT3; PSMA; PSCA; MUC1; MUC16; STEAP; CEA; TENB2; EphA receptors; EphB receptors; folate receptor; FOLR1; mesothelin; crypto; αᵥβ₆; integrins; VEGF, VEGFR; EGFR; fibroblast growth factor receptor (FGFR) (e.g., FGFR1, FGFR2, FGFR3, FGFR4); transferrin receptor; IRTA1; IRTA2; IRTA3; IRTA4; IRTA5; CD proteins such as CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80. CD81, CD103, CD105, CD123, CD134, CD137, CD138, CD152, guanylyl cyclase C (GCC), or an antibody which binds to one or more tumor-associated antigens or cell-surface receptors disclosed in U.S. Patent Application Publication No. 2008/0171040 or U.S. Patent Application Publication No. 2008/0305044 and are incorporated in their entirety by reference; erythropoietin; osteoinductive factors; immunotoxins; a bone morphogenetic protein (BMP); an interferon, such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the HIV envelope; transport proteins; homing receptors; addressins; regulatory proteins; integrins, such as CD11a, CD11b, CD11c, CD18, an ICAM, VLA-4 and VCAM; a tumor associated antigen such as HER2, HER3, or HER4 receptor; endoglin; c-Met; IGF1R; prostate antigens such as PCA3, PSA, PSGR, NGEP, PSMA, PSCA, TMEFF2, and STEAP1; LGR5; B7H4; and fragments of any of the above-listed polypeptides. In one embodiment, the antigen is not GCC.

Additionally, GM-CSF, which binds to myeloid cells can be used as a cell-binding agent to diseased cells from acute myelogenous leukemia. IL-2 which binds to activated T-cells can be used for prevention of transplant graft rejection, for therapy and prevention of graft-versus-host disease, and for treatment of acute T-cell leukemia. MSH, which binds to melanocytes, can be used for the treatment of melanoma, as can antibodies directed towards melanomas. Folic acid can be used to target the folate receptor expressed on ovarian and other tumors. Epidermal growth factor can be used to target squamous cancers such as lung and head and neck. Somatostatin can be used to target neuroblastomas and other tumor types.

Cancers of the breast and testes can be successfully targeted with estrogen (or estrogen analogues) or androgen (or androgen analogues) respectively as cell-binding agents.

The term “antibody,” as used herein, refers to any immunoglobulin, any immunoglobulin fragment, such as Fab, Fab′, F(ab′).sub.2, dsFv, sFv, minibodies, diabodies, tribodies, tetrabodies, probodies (Parham, J. Immunol., 131: 2895-2902 (1983); Spring et al. J. Immunol., 113: 470-478 (1974); Nisonoff et al. Arch. Biochem. Biophys., 89: 230-244 (1960), Kim et al., Mol. Cancer Ther., 7: 2486-2497 (2008), Carter, Nature Revs., 6: 343-357 (2006), U.S. Pat. No. 8,399,219), or immunoglobulin chimera, which can bind to an antigen on the surface of a cell (e.g., which contains a complementarity determining region (CDR)). Any suitable antibody can be used as the cell-binding agent. One of ordinary skill in the art will appreciate that the selection of an appropriate antibody will depend upon the cell population to be targeted. In this regard, the type and number of cell surface molecules (i.e., antigens) that are selectively expressed in a particular cell population (typically and preferably a diseased cell population) will govern the selection of an appropriate antibody for use in the inventive composition. Cell surface expression profiles are known for a wide variety of cell types, including tumor cell types, or, if unknown, can be determined using routine molecular biology and histochemistry techniques.

The antibody can be polyclonal or monoclonal, but is most preferably a monoclonal antibody. As used herein, “polyclonal” antibodies refer to heterogeneous populations of antibody molecules, typically contained in the sera of immunized animals. “Monoclonal” antibodies refer to homogenous populations of antibody molecules that are specific to a particular antigen. Monoclonal antibodies are typically produced by a single clone of B lymphocytes (“B cells”). Monoclonal antibodies may be obtained using a variety of techniques known to those skilled in the art, including standard hybridoma technology (see, e.g., Kohler and Milstein, Eur. J. Immunol., 5: 511-519 (1976), Harlow and Lane (eds.), Antibodies: A Laboratory Manual, CSH Press (1988), and C. A. Janeway et al. (eds.), Immunobiology, 5.sup.th Ed., Garland Publishing, New York, N.Y. (2001)). In brief, the hybridoma method of producing monoclonal antibodies typically involves injecting any suitable animal, typically and preferably a mouse, with an antigen (i.e., an “immunogen”). The animal is subsequently sacrificed, and B cells isolated from its spleen are fused with human myeloma cells. A hybrid cell is produced (i.e., a “hybridoma”), which proliferates indefinitely and continuously secretes high titers of an antibody with the desired specificity in vitro. Any appropriate method known in the art can be used to identify hybridoma cells that produce an antibody with the desired specificity. Such methods include, for example, enzyme-linked immunosorbent assay (ELISA), Western blot analysis, and radioimmunoas say. The population of hybridomas is screened to isolate individual clones, each of which secretes a single antibody species to the antigen. Because each hybridoma is a clone derived from fusion with a single B cell, all the antibody molecules it produces are identical in structure, including their antigen binding site and isotype. Monoclonal antibodies also may be generated using other suitable techniques including EBV-hybridoma technology (see, e.g., Haskard and Archer, J. Immunol. Methods, 74(2): 361-67 (1984), and Roder et al., Methods Enzymol., 121: 140-67 (1986)), bacteriophage vector expression systems (see, e.g., Huse et al., Science, 246: 1275-81 (1989)), or phage display libraries comprising antibody fragments, such as Fab and scFv (single chain variable region) (see, e.g., U.S. Pat. Nos. 5,885,793 and 5,969,108, and International Patent Application Publications WO 92/01047 and WO 99/06587).

The monoclonal antibody can be isolated from or produced in any suitable animal, but is preferably produced in a mammal, more preferably a mouse or human, and most preferably a human. Methods for producing an antibody in mice are well known to those skilled in the art and are described herein. With respect to human antibodies, one of ordinary skill in the art will appreciate that polyclonal antibodies can be isolated from the sera of human subjects vaccinated or immunized with an appropriate antigen. Alternatively, human antibodies can be generated by adapting known techniques for producing human antibodies in non-human animals such as mice (see, e.g., U.S. Pat. Nos. 5,545,806, 5,569,825, and 5,714,352, and U.S. Patent Application Publication No. 2002/0197266 A1).

While being the ideal choice for therapeutic applications in humans, human antibodies, particularly human monoclonal antibodies, typically are more difficult to generate than mouse monoclonal antibodies. Mouse monoclonal antibodies, however, induce a rapid host antibody response when administered to humans, which can reduce the therapeutic or diagnostic potential of the antibody-cytotoxic agent conjugate. To circumvent these complications, a monoclonal antibody preferably is not recognized as “foreign” by the human immune system.

To this end, phage display can be used to generate the antibody. In this regard, phage libraries encoding antigen-binding variable (V) domains of antibodies can be generated using standard molecular biology and recombinant DNA techniques (see, e.g., Sambrook et al. (eds.), Molecular Cloning, A Laboratory Manual, 3.sup.rd Edition, Cold Spring Harbor Laboratory Press, New York (2001)). Phages encoding a variable region with the desired specificity are selected for specific binding to the desired antigen, and a complete human antibody is reconstituted comprising the selected variable domain. Nucleic acid sequences encoding the reconstituted antibody are introduced into a suitable cell line, such as a myeloma cell used for hybridoma production, such that human antibodies having the characteristics of monoclonal antibodies are secreted by the cell (see, e.g., Janeway et al., supra, Huse et al., supra, and U.S. Pat. No. 6,265,150). Alternatively, monoclonal antibodies can be generated from mice that are transgenic for specific human heavy and light chain immunoglobulin genes. Such methods are known in the art and described in, for example, U.S. Pat. Nos. 5,545,806 and 5,569,825, and Janeway et al., supra.

Most preferably the antibody is a humanized antibody. As used herein, a “humanized” antibody is one in which the complementarity-determining regions (CDR) of a mouse monoclonal antibody, which form the antigen binding loops of the antibody, are grafted onto the framework of a human antibody molecule. Owing to the similarity of the frameworks of mouse and human antibodies, it is generally accepted in the art that this approach produces a monoclonal antibody that is antigenically identical to a human antibody but binds the same antigen as the mouse monoclonal antibody from which the CDR sequences were derived. Methods for generating humanized antibodies are well known in the art and are described in detail in, for example, Janeway et al., supra, U.S. Pat. Nos. 5,225,539, 5,585,089 and 5,693,761, European Patent No. 0239400 B1, and United Kingdom Patent No. 2188638. Humanized antibodies can also be generated using the antibody resurfacing technology described in U.S. Pat. No. 5,639,641 and Pedersen et al., J. Mol. Biol., 235: 959-973 (1994). While the antibody employed in the conjugate of the inventive composition most preferably is a humanized monoclonal antibody, a human monoclonal antibody and a mouse monoclonal antibody, as described above, are also within the scope of the invention.

Antibody fragments that have at least one antigen binding site, and thus recognize and bind to at least one antigen or receptor present on the surface of a target cell, also are within the scope of the invention. In this respect, proteolytic cleavage of an intact antibody molecule can produce a variety of antibody fragments that retain the ability to recognize and bind antigens. For example, limited digestion of an antibody molecule with the protease papain typically produces three fragments, two of which are identical and are referred to as the Fab fragments, as they retain the antigen binding activity of the parent antibody molecule. Cleavage of an antibody molecule with the enzyme pepsin normally produces two antibody fragments, one of which retains both antigen-binding arms of the antibody molecule, and is thus referred to as the F(ab′).sub.2 fragment. Reduction of a F(ab′).sub.2 fragment with dithiothreitol or mercaptoethylamine produces a fragment referred to as a Fab′ fragment. A single-chain variable region fragment (sFv) antibody fragment, which consists of a truncated Fab fragment comprising the variable (V) domain of an antibody heavy chain linked to a V domain of a light antibody chain via a synthetic peptide, can be generated using routine recombinant DNA technology techniques (see, e.g., Janeway et al., supra). Similarly, disulfide-stabilized variable region fragments (dsFv) can be prepared by recombinant DNA technology (see, e.g., Reiter et al., Protein Engineering, 7: 697-704 (1994)). Antibody fragments in the context of the invention, however, are not limited to these exemplary types of antibody fragments. Any suitable antibody fragment that recognizes and binds to a desired cell surface receptor or antigen can be employed. Antibody fragments are further described in, for example, Parham, J. Immunol., 131: 2895-2902 (1983), Spring et al., J. Immunol., 113: 470-478 (1974), and Nisonoff et al., Arch. Biochem. Biophys., 89: 230-244 (1960). Antibody-antigen binding can be assayed using any suitable method known in the art, such as, for example, radioimmunoas say (RIA), ELISA, Western blot, immunoprecipitation, and competitive inhibition assays (see, e.g., Janeway et al., supra, and U.S. Patent Application Publication No. 2002/0197266 A1).

In addition, the antibody can be a chimeric antibody or an antigen binding fragment thereof. By “chimeric” it is meant that the antibody comprises at least two immunoglobulins, or fragments thereof, obtained or derived from at least two different species (e.g., two different immunoglobulins, such as a human immunoglobulin constant region combined with a murine immunoglobulin variable region). The antibody also can be a domain antibody (dAb) or an antigen binding fragment thereof, such as, for example, a camelid antibody (see, e.g., Desmyter et al., Nature Struct. Biol., 3: 752, (1996)), or a shark antibody, such as, for example, a new antigen receptor (IgNAR) (see, e.g., Greenberg et al., Nature, 374: 168 (1995), and Stanfield et al., Science, 305: 1770-1773 (2004)).

Any suitable antibody can be used in the context of the invention. For example, the monoclonal antibody J5 is a murine IgG2a antibody that is specific for Common Acute Lymphoblastic Leukemia Antigen (CALLA) (Ritz et al., Nature, 283: 583-585 (1980)), and can be used to target cells that express CALLA (e.g., acute lymphoblastic leukemia cells). The monoclonal antibody MY9 is a murine IgG1 antibody that binds specifically to the CD33 antigen (Griffin et al., Leukemia Res., 8: 521 (1984)), and can be used to target cells that express CD33 (e.g., acute myelogenous leukemia (AML) cells). In certain embodiments, the MY9 antibody has the N-terminal or C-terminal residue removed.

Similarly, the monoclonal antibody anti-B4 (also referred to as B4) is a murine IgG1 antibody that binds to the CD19 antigen on B cells (Nadler et al., J. Immunol., 131: 244-250 (1983)), and can be used to target B cells or diseased cells that express CD19 (e.g., non-Hodgkin's lymphoma cells and chronic lymphoblastic leukemia cells). N901 is a murine monoclonal antibody that binds to the CD56 (neural cell adhesion molecule) antigen found on cells of neuroendocrine origin, including small cell lung tumor, which can be used in the conjugate to target drugs to cells of neuroendocrine origin. The J5, MY9, and B4 antibodies preferably are resurfaced or humanized prior to their use as part of the conjugate. Resurfacing or humanization of antibodies is described in, for example, Roguska et al., Proc. Natl. Acad. Sci. USA, 91: 969-73 (1994).

In addition, the monoclonal antibody C242 binds to the CanAg antigen (see, e.g., U.S. Pat. No. 5,552,293), and can be used to target the conjugate to CanAg expressing tumors, such as colorectal, pancreatic, non-small cell lung, and gastric cancers. HuC242 is a humanized form of the monoclonal antibody C242 (see, e.g., U.S. Pat. No. 5,552,293). The hybridoma from which HuC242 is produced is deposited with ECACC identification Number 90012601. HuC242 can be prepared using CDR-grafting methodology (see, e.g., U.S. Pat. Nos. 5,585,089, 5,693,761, and 5,693,762) or resurfacing technology (see, e.g., U.S. Pat. No. 5,639,641). HuC242 can be used to target the conjugate to tumor cells expressing the CanAg antigen, such as, for example, colorectal, pancreatic, non-small cell lung, and gastric cancer cells.

To target ovarian cancer and prostate cancer cells, an anti-MUC1 antibody can be used as the cell-binding agent in the conjugate. Anti-MUC1 antibodies include, for example, anti-HMFG-2 (see, e.g., Taylor-Papadimitriou et al., Int. J. Cancer, 28: 17-21 (1981)), hCTMO1 (see, e.g., van H of et al., Cancer Res., 56: 5179-5185 (1996)), and DS6. Prostate cancer cells also can be targeted with the conjugate by using an anti-prostate-specific membrane antigen (PSMA) as the cell-binding agent, such as J591 (see, e.g., Liu et al., Cancer Res., 57: 3629-3634 (1997)). Moreover, cancer cells that express the Her2 antigen, such as breast, prostate, and ovarian cancers, can be targeted with the conjugate by using anti-Her2 antibodies, e.g., trastuzumab, as the cell-binding agent. Cells that express epidermal growth factor receptor (EGFR) and variants thereof, such as the type III deletion mutant, EGFRvIII, can be targeted with the conjugate by using anti-EGFR antibodies. Anti-EGFR antibodies are described in International Patent Application Nos. PCT/US11/058,385 and PCT/US11/058,378. Anti-EGFRvIII antibodies are described in U.S. Pat. Nos. 7,736,644 and 7,628,986 and U.S. Application Publications 2010/0111979, 2009/0240038, 2009/0175887, 2009/0156790, and 2009/0155282. Anti-IGF-IR antibodies that bind to insulin-like growth factor receptor, such as those described in U.S. Pat. No. 7,982,024, also can be used in the conjugate. Antibodies that bind to CD27L, Cripto, CD138, CD38, EphA2, integrins, CD37, folate, CD20, PSGR, NGEP, PSCA, TMEFF2, STEAP1, endoglin, and Her3 also can be used in the conjugate.

In one embodiment, the antibody is selected from the group consisting of huN901, huMy9-6, huB4, huC242, an anti-HER2 antibody (e.g., trastuzumab), bivatuzumab, sibrotuzumab, rituximab, huDS6, anti-mesothelin antibodies described in International Patent Application Publication WO 2010/124797 (such as MF-T), anti-cripto antibodies described in U.S. Patent Application Publication 2010/0093980 (such as huB3F6), anti-CD138 antibodies described in U.S. Patent Application Publication 2007/0183971 (such as huB-B4), anti-EGFR antibodies described in International Patent Application Nos. PCT/US11/058,385 and PCT/US11/058,378 (such as EGFR-7), anti-EGFRvIII antibodies described U.S. Pat. Nos. 7,736,644 and 7,628,986 and U.S. Patent Application Publications 2010/0111979, 2009/0240038, 2009/0175887, 2009/0156790 and 2009/0155282, humanized EphA2 antibodies described in International Patent Application Publications WO 2011/039721 and WO 2011/039724 (such as 2H11R35R74); anti-CD38 antibodies described in International Patent Application Publication WO 2008/047242 (such as hu38SB19), anti-folate antibodies described in International Patent Application Publication WO 2011/106528, and U.S. Patent Application Publication 2012/0009181 (e.g., huMov19); anti-IGF1R antibodies described in U.S. Pat. Nos. 5,958,872, 6,596,743, and 7,982,024; anti-CD37 antibodies described in U.S. Patent Application Publication 2011/0256153 (e.g., huCD37-3); anti-integrin αᵥβ₆ antibodies described in U.S. Application Publication 2006/0127407 (e.g., CNTO95); and anti-Her3 antibodies described in International Patent Application Publication WO 2012/019024. In one embodiment, the cell-binding agent is an antibody or an antigen binding fragment thereof that binds to FGFR2 (e.g., those described in US2014/030820, the entire teachings of which is incorporated herein by reference). In another embodiment, the cell-binding agent is an antibody or an antigen binding fragment thereof that binds to FGFR2 and FGFR4 (e.g., those described in US 2014/301946, the entire teachings of which is incorporated herein by reference).

Particularly preferred antibodies are humanized monoclonal antibodies described herein. Examples include, but are not limited to, huN901, huMy9-6, huB4, huC242, a humanized monoclonal anti-Her2 antibody (e.g., trastuzumab), bivatuzumab, sibrotuzumab, CNTO95, huDS6, and rituximab (see, e.g., U.S. Pat. Nos. 5,639,641 and 5,665,357, U.S. Provisional Patent Application No. 60/424,332 (which is related to U.S. Pat. No. 7,557,189), International (PCT) Patent Application Publication WO 02/16401, Pedersen et al., supra, Roguska et al., supra, Liu et al., supra, Nadler et al., supra, Colomer et al., Cancer Invest., 19: 49-56 (2001), Heider et al., Eur. J. Cancer, 31A: 2385-2391 (1995), Welt et al., J. Clin. Oncol., 12: 1193-1203 (1994), and Maloney et al., Blood, 90: 2188-2195 (1997)). Other humanized monoclonal antibodies are known in the art and can be used in connection with the invention.

In one embodiment, the cell-binding agent is huMy9-6, or other related antibodies, which are described in U.S. Pat. Nos. 7,342,110 and 7,557,189 (incorporated herein by reference).

In another embodiment, the cell-binding agent is an anti-folate receptor antibody described in U.S. Pat. Nos. 8,557,966 and 9,133,275. The teachings of each of these patents is incorporated herein by reference in its entirety.

In another embodiment, the cell-binding agent is an humanized anti-folate antibody or antigen binding fragment thereof that specifically binds a human folate receptor 1 (FOLR1), wherein the antibody comprises: (a) a heavy chain CDR1 comprising GYFMN (SEQ ID NO:1); a heavy chain CDR2 comprising RIHPYDGDTFYNQXaaᵢFXaa₂Xaa₃ (SEQ ID NO:2); and a heavy chain CDR3 comprising YDGSRAMDY (SEQ ID NO:3); and (b) a light chain CDR1 comprising KASQSVSFAGTSLMH (SEQ ID NO:4); a light chain CDR2 comprising RASNLEA (SEQ ID NO:5); and a light chain CDR3 comprising QQSREYPYT (SEQ ID NO:6); wherein Xaaᵢ is selected from K, Q, H, and R; Xaa₂ is selected from Q, H, N, and R; and Xaa₃ is selected from G, E, T, S, A, and V. Preferably, the heavy chain CDR2 sequence comprises RIHPYDGDTFYNQKFQG (SEQ ID NO:7).

In another embodiment, the anti-folate antibody is a humanized antibody or antigen binding fragment thereof that specifically binds the human folate receptor 1 comprising the heavy chain having the amino acid sequence of

(SEQ ID NO: 8)
QVQLVQSGAEVVKPGASVKISCKASGYTFTGYFMNWVKQSPGQSLEWIGR
IHPYDGDTFYNQKFQGKATLTVDKSSNTAHMELLSLTSEDFAVYYCTRYD
GSRAMDYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK.

In another embodiment, the anti-folate antibody is a humanized antibody or antigen binding fragment thereof encoded by the plasmid DNA deposited with the ATCC on Apr. 7, 2010 and having ATCC deposit nos. PTA-10772 and PTA-10773 or 10774.

In another embodiment, the anti-folate antibody is a humanized antibody or antigen binding fragment thereof comprising a heavy chain variable domain at least about 90%, 95%, 99% or 100% identical to QVQLVQSGAEVVKPGASVKISCKASGYTFTGYFMNWVKQSPGQSLEWIGRIHPYDG DTFYNQKFQGKATLTVDKSSNTAHMELLSLTSEDFAVYYCTRYDGSRAMDYWGQG TTVTVSS (SEQ ID NO:24), and a light chain variable domain at least about 90%, 95%, 99% or 100% identical to

(SEQ ID NO: 9)
DIVLTQSPLSLAVSLGQPAIISCKASQSVSFAGTSLMHWYHQKPGQQPRL
LIYRASNLEAGVPDRFSGSGSKTDFTLNISPVEAEDAATYYCQQSREYPY
TFGGGTKLEIKR;
or
(SEQ ID NO: 10)
DIVLTQSPLSLAVSLGQPAIISCKASQSVSFAGTSLMHWYHQKPGQQPRL
LIYRASNLEAGVPDRFSGSGSKTDFTLTISPVEAEDAATYYCQQSREYPY
TFGGGTKLEIKR.

In one embodiment, the cell-binding agent is an antibody or an antigen binding fragment thereof that specifically binds to GCC. In one embodiment, the antibody or an antigen binding fragment thereof comprises CDR sequences of SEQ ID NOs: 11-16. In one embodiment, the anti-GCC antibody has VH and VL sequences that are at least 95% identical to SEQ ID NO:17 and SEQ ID NO:18, respectively. In another embodiment, the anti-GCC antibody has VH and VL sequences that are SEQ ID NO:17 and SEQ ID NO:18, respectively. In yet another embodiment, the anti-GCC antibody comprises a heavy chain amino acid sequence of SEQ ID NO:19 and a light chain amino acid sequence of SEQ ID NO:20. In one embodiment, the anti-GCC antibody comprises a heavy chain amino acid sequence that replace ELLG in the heavy chain of IgG1 (SEQ ID NO:19), which are important for binding FcγRIIIb, with PVA; and a light chain amino acid sequence of SEQ ID NO:20,

| | | |
|---|---|---|
| VHCDR1 | SEQ ID NO: 11 | GYYWS |
| VHCDR2 | SEQ ID NO: 12 | EINHRGNTNDNPSLKS |
| VHCDR3 | SEQ ID NO: 13 | ERGYTYGNFDH |
| VLCDR1 | SEQ ID NO: 14 | RASQSVSRNLA |
| VLCDR2 | SEQ ID NO: 15 | GASTRAT |
| VLCDR3 | SEQ ID NO: 16 | QQYKTWPRT |
| 5F9 VH | SEQ ID NO: 17 | QVQLQQWGAGLLKPSETLSLTCAVFGGSFSGYYWSWIR |
| | | QPPGKGLEWIGEINHRGNTNDNPSLKSRVTISVDTSKNQF |
| | | ALKLSSVTAADTAVYYCARERGYTYGNFDHWGQGTLV |
| | | TVSS |
| 5F9 VL | SEQ ID NO: 18 | EIVMTQSPATLSVSPGERATLSCRASQSVSRNLAWYQQK |
| | | PGQAPRLLIYGASTRATGIPARFSGSGSGTEFTLTIGSLQS |
| | | EDFAVYYCQQYKTWPRTFGQGTNVEIK |
| 5F9/hIgG1 | SEQ ID NO: 19 | MGWSCIILFLVATATGVHSQVQLQQWGAGLLKPSETLSLTC |
| heavy chain | | AVFGGSFS [symbol] WIRQPPGKGLEWIG [symbol] |
| | | [symbol] RVTISVDTSKNQFALKLSSVTAADTAVYYCAR [symbol] |
| | | [symbol] WGQGTLVTVSSASTKGPSVFPLAPSSKSTSGGTAALGCLV |
| | | KDYFPEPVTVSWNSGALTSGVHTFPAVLQSSGLYSLSSVVTVP |
| | | SSSLGTQTYICNVNHKPSNTKVDKKVEPKSCDKTHTCPPCPAP |
| | | ELLGGPSVFLFPPKPKDTLMISRTPEVTCVVVDVSHEDPEVKFN |
| | | WYVDGVEVHNAKTKPREEQYNSTYRVVSVLTVLHQDWLNG |
| | | KEYKCKVSNKALPAPIEKTISKAKGQPREPQVYTLPPSRDELTK |
| | | NQVSLTCLVKGFYPSDIAVEWESNGQPENNYKTTPPVLDSDGS |
| | | FFLYSKLTVDKSRWQQGNVFSCSVMHEALHNHYTQKSLSLSP |
| | | GK |
| 5F9/hKappa | SEQ ID NO: 20 | MGWSCIILFLVATATGVHSEIVMTQSPATLSVSPGERATLSC [symbol] |
| light chain | | [symbol] WYQQKPGQAPRLLIY [symbol] GIPARFSGSG |
| | | SGTEFTLTIGSLQSEDFAVYYC [symbol] FGQGTNVEIKR |
| | | TVAAPSVFIFPPSDEQLKSGTASVVCLLNNFYPREAKVQWKVD |
| | | NALQSGNSQESVTEQDSKDSTYSLSSTLTLSKADYEKHKVYA |
| | | CEVTHQGLSSPVTKSFNRGEC |

In one embodiment, the cell-binding agent is not an anti-GCC antibody or an antigen binding fragment thereof.

While the cell-binding agent preferably is an antibody, the cell-binding agent also can be a non-antibody molecule. Suitable non-antibody molecules include, for example, interferons (e.g., alpha, beta, or gamma interferon), lymphokines (e.g., interleukin 2 (IL-2), IL-3, IL-4, or IL-6), hormones (e.g., insulin), growth factors (e.g., EGF, TGF-alpha, FGF, and VEGF), colony-stimulating factors (e.g., G-CSF, M-CSF, and GM-CSF (see, e.g., Burgess, Immunology Today, 5: 155-158 (1984)), somatostatin, and transferrin (see, e.g., O'Keefe et al., J. Biol. Chem., 260: 932-937 (1985)). For example, GM-CSF, which binds to myeloid cells, can be used as a cell-binding agent to target acute myelogenous leukemia cells.

In addition, IL-2, which binds to activated T-cells, can be used for prevention of transplant graft rejection, for therapy and prevention of graft-versus-host disease, and for treatment of acute T-cell leukemia. Epidermal growth factor (EGF) can be used to target squamous cancers such as lung cancer and head and neck cancer. Somatostatin can be used to target neuroblastoma cells and other tumor cell types.

In certain embodiments, the cell-binding agent (e.g., antibody) that can be used in the methods of the present invention comprises a free amine —NH₂ group (e.g., epsilon amino group on one or more lysine residues) that can form a covalent bond with the cytotoxic agent or the cytotoxic agent-linker compound having an amine-reactive group.

### Cytotoxic Agent or Cytotoxic Agent-Linker Compounds

A “cytotoxic agent,” as used herein, refers to any compound that results in the death of a cell, induces cell death, or decreases cell viability. In one embodiment, the cytotoxic agent is a benzodiazepine dimer compound. In another embodiment, the cytotoxic agent is a indolinobenzodiazepine dimer compound. Preferably, the indolinobenzodiazepine dimer compound has an amine-reactive group that can form a covalent bond with the amine group on the cell-binding agent (e.g., lysine amine group).

In certain embodiments, the cytotoxic agent can react with a linker having an amine-reactive group to form the cytotoxic agent-linker compound having the amine-reactive group attached thereto. The resulting cytotoxic agent-linker compound can then react with the cell-binding agent to form the cell-binding agent-cytotoxic agent conjugate.

As used herein, the term “amine-reactive group” refers to functional group that can readily react with an amine group to form a covalent bond. In one embodiment, the amine-reactive group is a reactive ester group. Examples of reactive ester groups include, but are not limited to, N-hydroxysuccinimde ester, N-hydroxy sulfosuccinimide ester, nitrophenyl (e.g., 2 or 4-nitrophenyl) ester, dinitrophenyl (e.g., 2,4-dinitrophenyl) ester, sulfo-tetraflurophenyl (e.g., 4-sulfo-2,3,5,6-tetrafluorophenyl) ester, and pentafluorophenyl ester.

In one embodiment, the reactive ester group is N-hydroxysuccinimide ester or N-hydroxysulfosuccinimide ester.

In a 31^{st} specific embodiment, for methods of present invention described herein (e.g., the method described in the first, second or third embodiment or the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th} or 30^{th} specific embodiment), the cytotoxic agent or the cytotoxic agent-linker compound is represented by one of the following structural formulas:

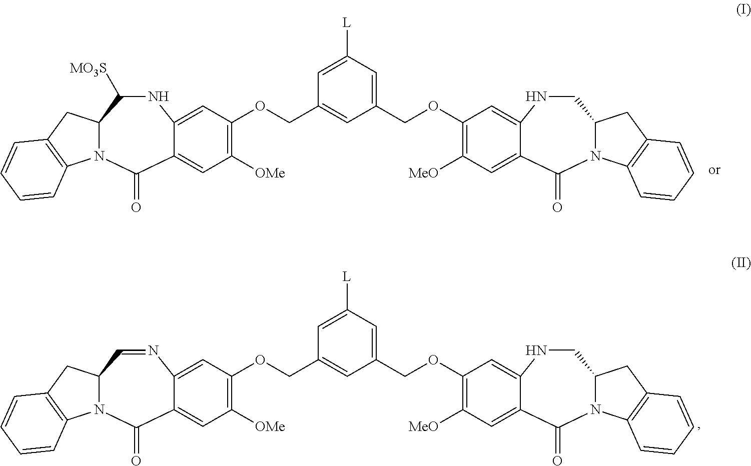

or a pharmaceutically acceptable salt thereof, wherein:
L is represented by the following formula:

—NR₅—P—C(═O)—(CRₐR_{b})ₘ—C(═O)E (A1); or

—NR₅—P—C(═O)—(CRₐR_{b})ₘ—S—Z^{s1} (A3);
wherein:
   R₅ is —H or a (C₁-C₃)alkyl;
   P is an amino acid residue or a peptide containing between 2 to 20 amino acid residues;
   Rₐ and R_{b}, for each occurrence, are each independently —H, (C₁-C₃)alkyl, or a charged substituent or an ionizable group Q (preferably Q is —SO₃M);
   m is an integer from 1 to 6; and
   Z^{s1} is selected from any one of the following formulas:

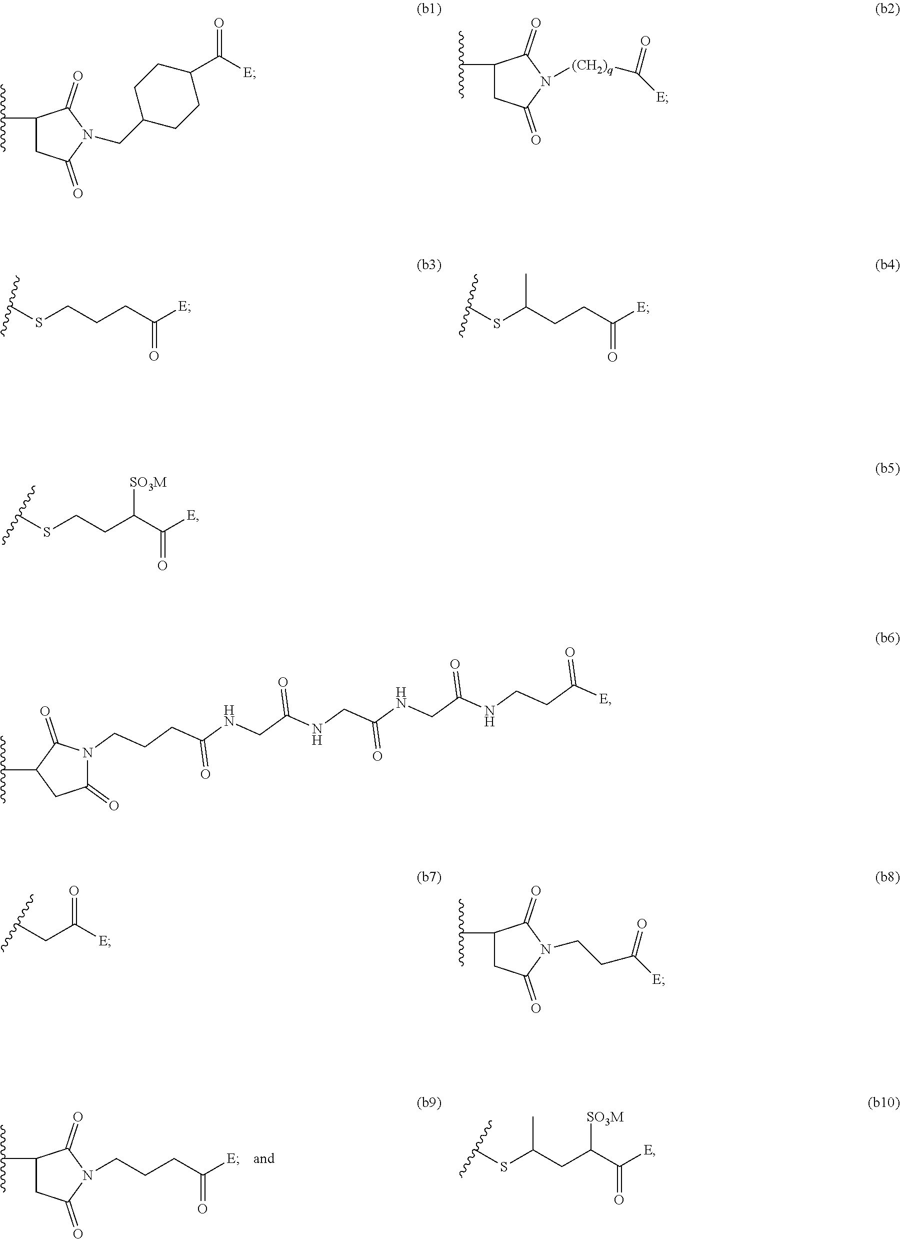

wherein:
q is an integer from 1 to 5;
M is —H or a cation; and
—C(═O)E represents a reactive ester group.

In a 32^{nd} specific embodiment, for compounds of formula (I) or (II) described above, Rₐ and R_{b} are both H; and R₅ is H or Me; and the remainder variables are as described in the 31^{st} specific embodiment.

In a 33^{rd} specific embodiment, for compounds of formula (I) or (II) described above, P is a peptide containing 2 to 5 amino acid residues; and the remainder variables are as described in the 31^{st} or 32^{nd} specific embodiment. In one embodiment, the peptide is cleavable by a protease, preferably cleavable by a protease expressed in tumor tissue. In another embodiment, P is selected from Gly-Gly-Gly, Ala-Val, Val-Ala, Val-Cit, Val-Lys, Phe-Lys, Lys-Lys, Ala-Lys, Phe-Cit, Leu-Cit, Ile-Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO:21), β-Ala-Leu-Ala-Leu (SEQ ID NO:22), Gly-Phe-Leu-Gly (SEQ ID NO:23), Val-Arg, Arg-Val, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, and Met-Ala. Preferably, P is Gly-Gly-Gly, Ala-Val, Ala-Ala, Ala-D-Ala, D-Ala-Ala, or D-Ala-D-Ala.

In a 34^{th} specific embodiment, for methods of present invention described herein (e.g., the method described in the first, second or third embodiment or the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27th 28^{th}, 29th or 30th specific embodiment), the cytotoxic agent or the cytotoxic agent-linker compound is represented by one of the following structural formulas:

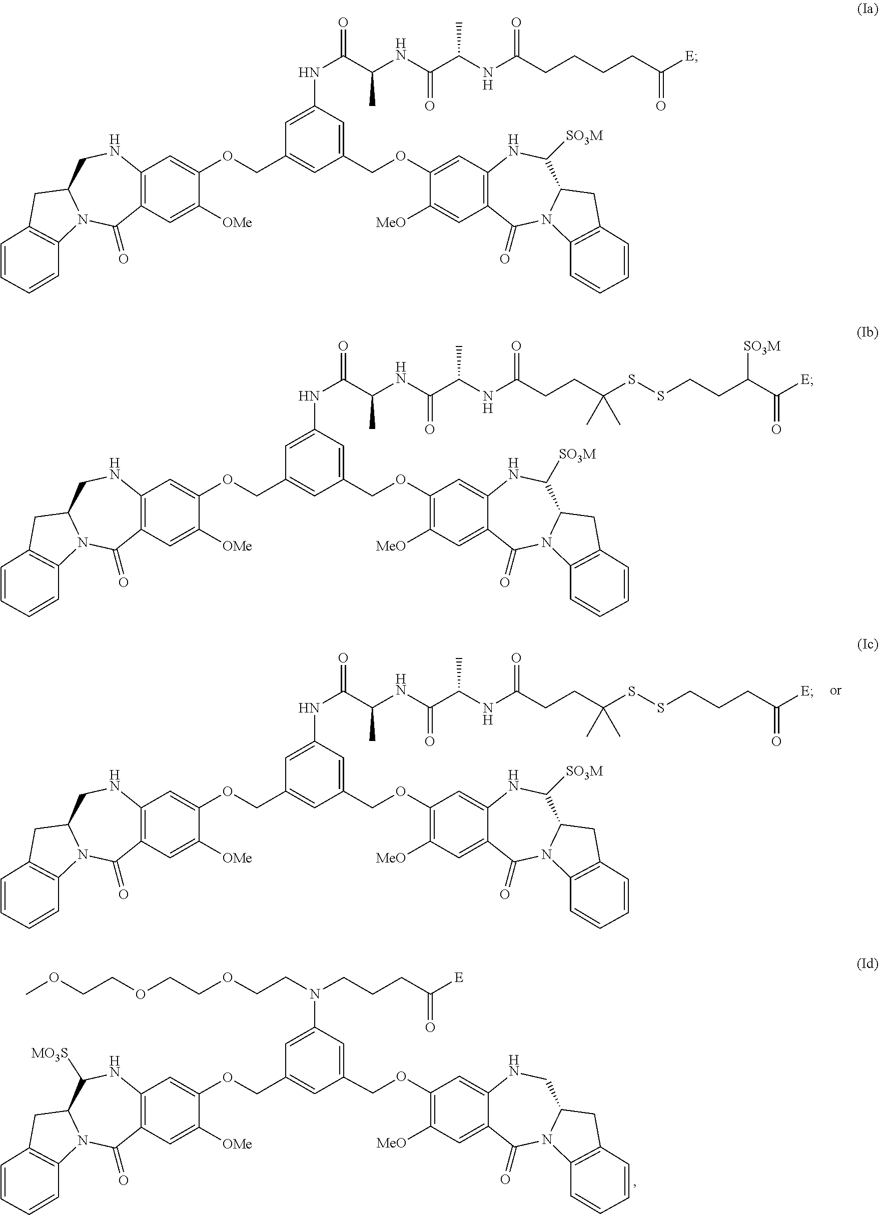

or a pharmaceutically acceptable salt thereof.

In a 35^{th} specific embodiment, for methods of present invention described herein (e.g., the method described in the first, second or third embodiment or the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th} or 30^{th} specific embodiment), the cytotoxic agent or the cytotoxic agent-linker compound is represented by one of the following structural formulas:

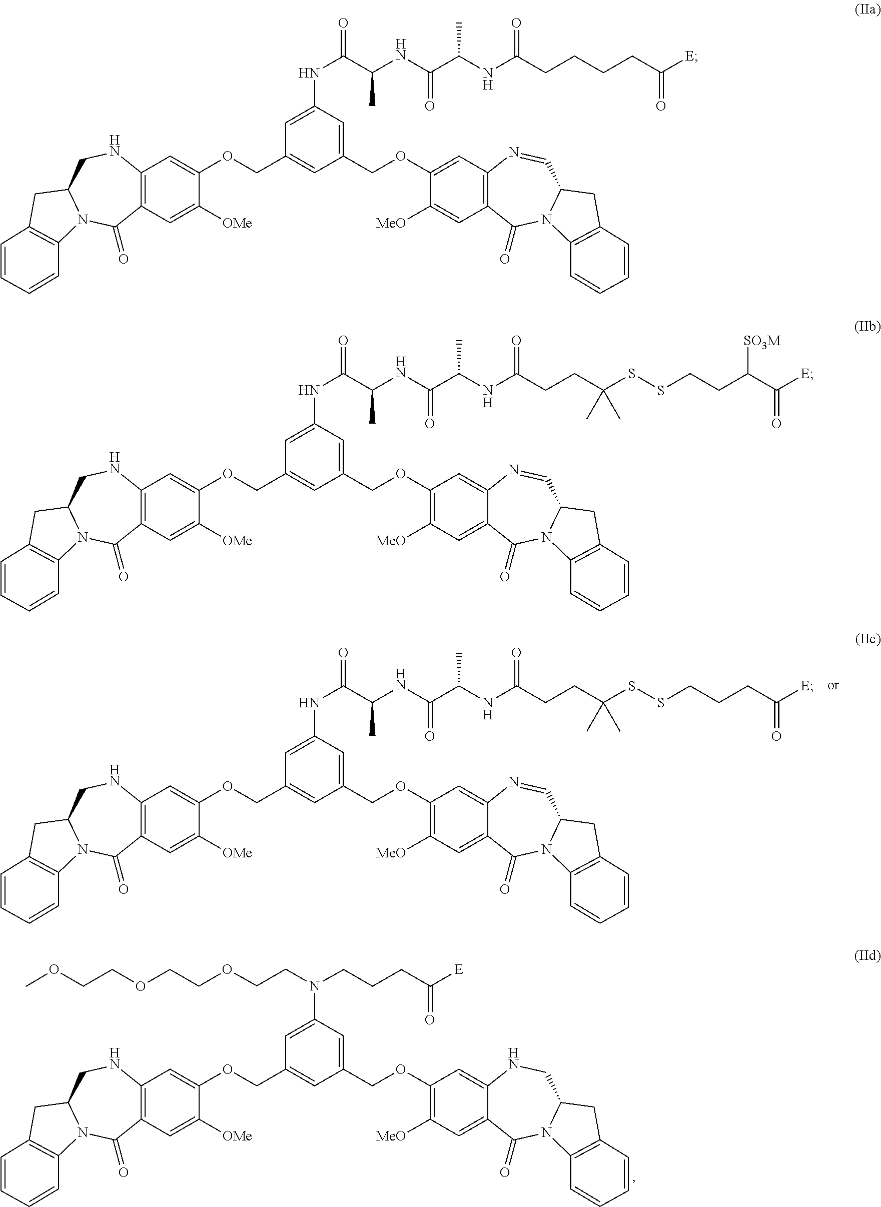

or a pharmaceutically acceptable salt thereof.

In one embodiment, for compounds described herein (e.g., the compounds described in the 31^{st}, 32^{nd}, 33^{rd}, 34^{th} or 35^{th} specific embodiment), the reactive ester group represented by —C(═O)E selected from N-hydroxysuccinimide ester, N-hydroxy sulfosuccinimide ester, nitrophenyl (e.g., 2 or 4-nitrophenyl) ester, dinitrophenyl (e.g., 2,4-dinitrophenyl) ester, sulfo-tetraflurophenyl (e.g., 4-sulfo-2,3,5,6-tetrafluorophenyl) ester, and pentafluorophenyl ester. More specifically, the reactive ester group is represented by the following formula:

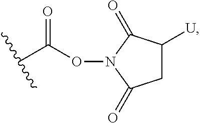

wherein U is H or —SO₃M. Even more specifically, the reactive ester group is represented by the following formula:

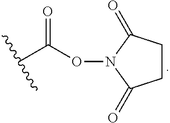 [SMILES: *C(=O)ON1C(=O)CCC1=O]

In a 36^{th} specific embodiment, for methods of present invention described herein (e.g., the method described in the first, second or third embodiment or the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th} or 30^{th} specific embodiment), the cytotoxic agent or the cytotoxic agent-linker compound is represented by the following structural formula:

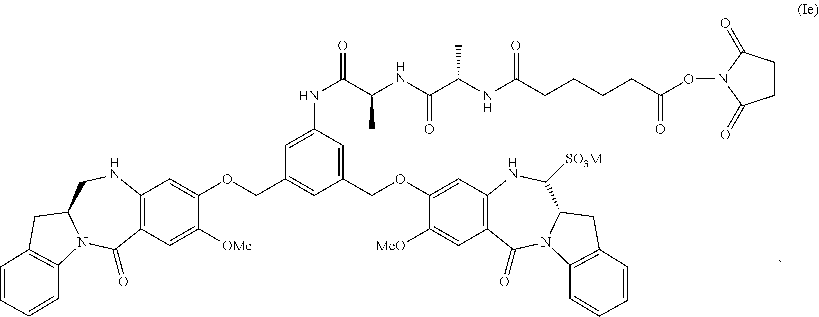

or a pharmaceutically acceptable salt thereof.

In one embodiment, for the method described in the 36^{th} embodiment, the compound of structural formula (Ie) is prepared by reacting the compound of (IIe) with a sulfonating agent. In a specific embodiment, the sulfonating agent is NaHSO₃ or KHSO₃. In another specific embodiment, for the method described in the 36^{th} embodiment, the compound of structural formula (Ie) is prepared by reacting the compound of (IIe) with a sulfonating agent in situ without purification before the the compound of structural formula (Ie) is reacted with the cell-binding agent. In one embodiment, the sulfonation reaction between the compound of formula (IIe) and the sulfonating agent (e.g., NaHSO₃ or KHSO₃) is carried out in an aqueous solution at a pH of 1.9 to 5.0, 2.9 to 4.0, 2.9 to 3.7, 3.1 to 3.5, 3.2 to 3.4. In a specific embodiment, the sulfonation reaction is carried out in an aqueous solution at pH 3.3. In one embodiment, the sulfonation reaction is carried out in dimethylacetamide (DMA) and water.

In a 37^{th} specific embodiment, for methods of present invention described herein (e.g., the method described in the first, second or third embodiment or the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th} or 30^{th} specific embodiment), the cytotoxic agent or the cytotoxic agent-linker compound is represented by the following structural formula:

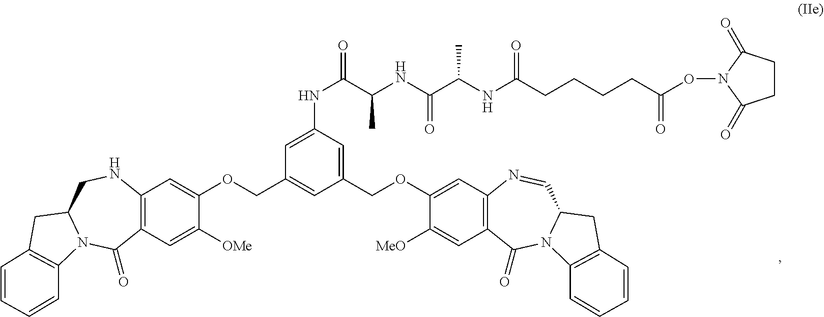 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)ON3C(=O)CCC3=O)c1)N=C[C@@H]1Cc3ccccc3N1C2=O]

or a pharmaceutically acceptable salt thereof.

In a 38^{th} specific embodiment, for methods of present invention described herein (e.g., the method described in the first, second or third embodiment or the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th} or 30^{th} specific embodiment), the cytotoxic agent or the cytotoxic agent-linker compound is represented by one of the following structural formulas:

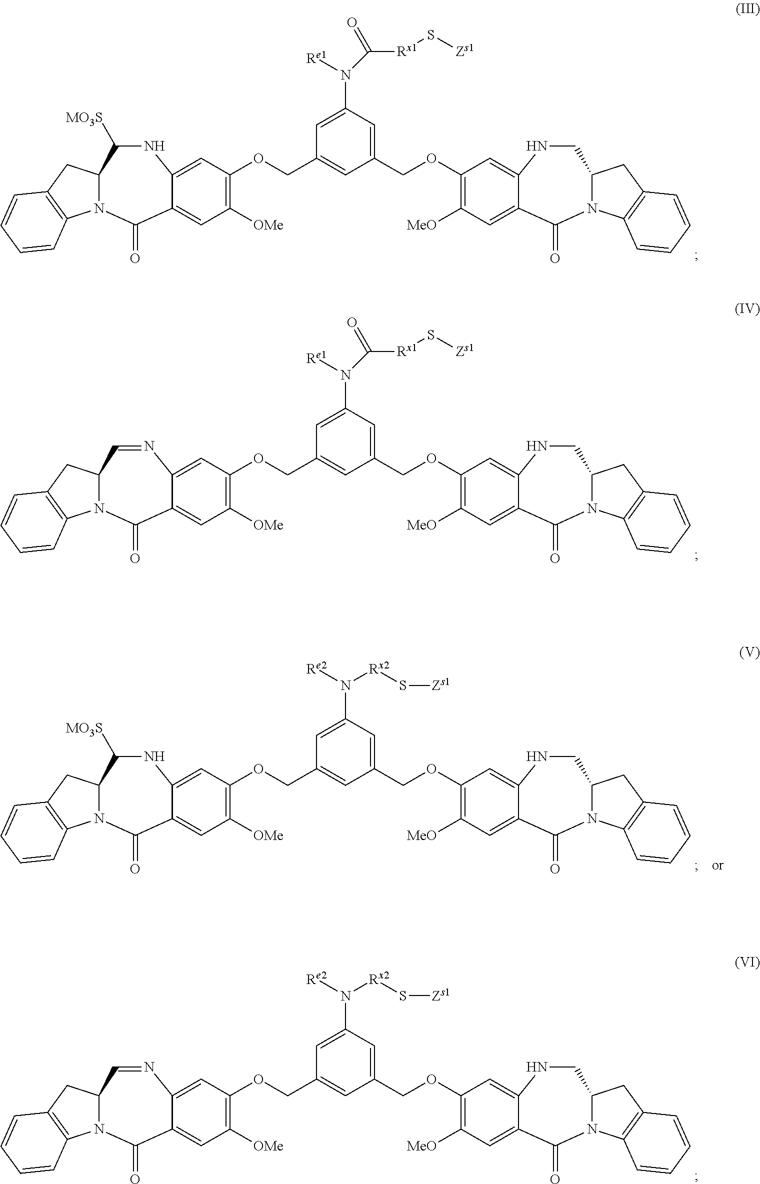

or a pharmaceutically acceptable salt thereof, wherein:
R^{x1} and R^{x2} are independently (C₁-C₆)alkyl;
R^{e1} is —H or a (C₁-C₆)alkyl;
R^{e2} is —(CH₂—CH₂—O)ₙ—R^{k};
n is an integer from 2 to 6;
R^{k} is —H or -Me;
Z^{s1} is selected from any one of the following formulas:

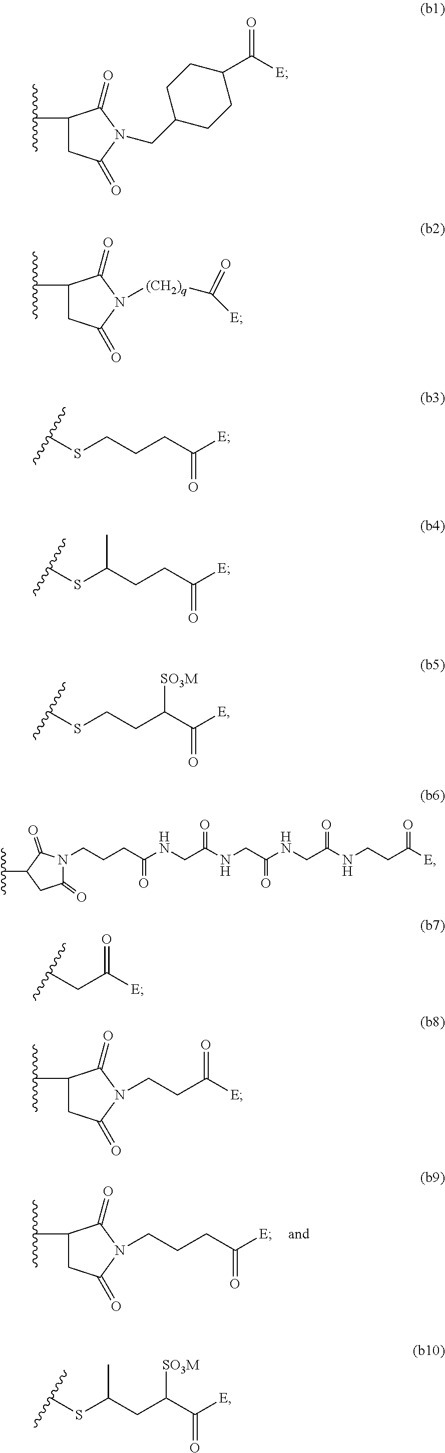

wherein:
q is an integer from 1 to 5;
M is —H or a cation; and
—C(═O)E represents a reactive ester group.

In a 39^{th} specific embodiment, for the compound represented by structural formulas (III), (IV), (V) and (VI), R^{e1} is H or Me; R^{x1} and R^{x2} are independently —(CH₂)ₚ—(CR^{f}R^{g})—, wherein R^{f} and R^{g} are each independently —H or a (C₁-C₄)alkyl; and p is 0, 1, 2 or 3; and the remaining variables are as described above in the 38^{th} specific embodiment. Preferably, R^{f} and R^{g} are the same or different, and are selected from —H and -Me.

In a 40^{th} specific embodiment, for methods of present invention described herein (e.g., the method described in the first, second or third embodiment or the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th} or 30^{th} specific embodiment), the cytotoxic agent or the cytotoxic agent-linker compound is represented by one of the following formulas:

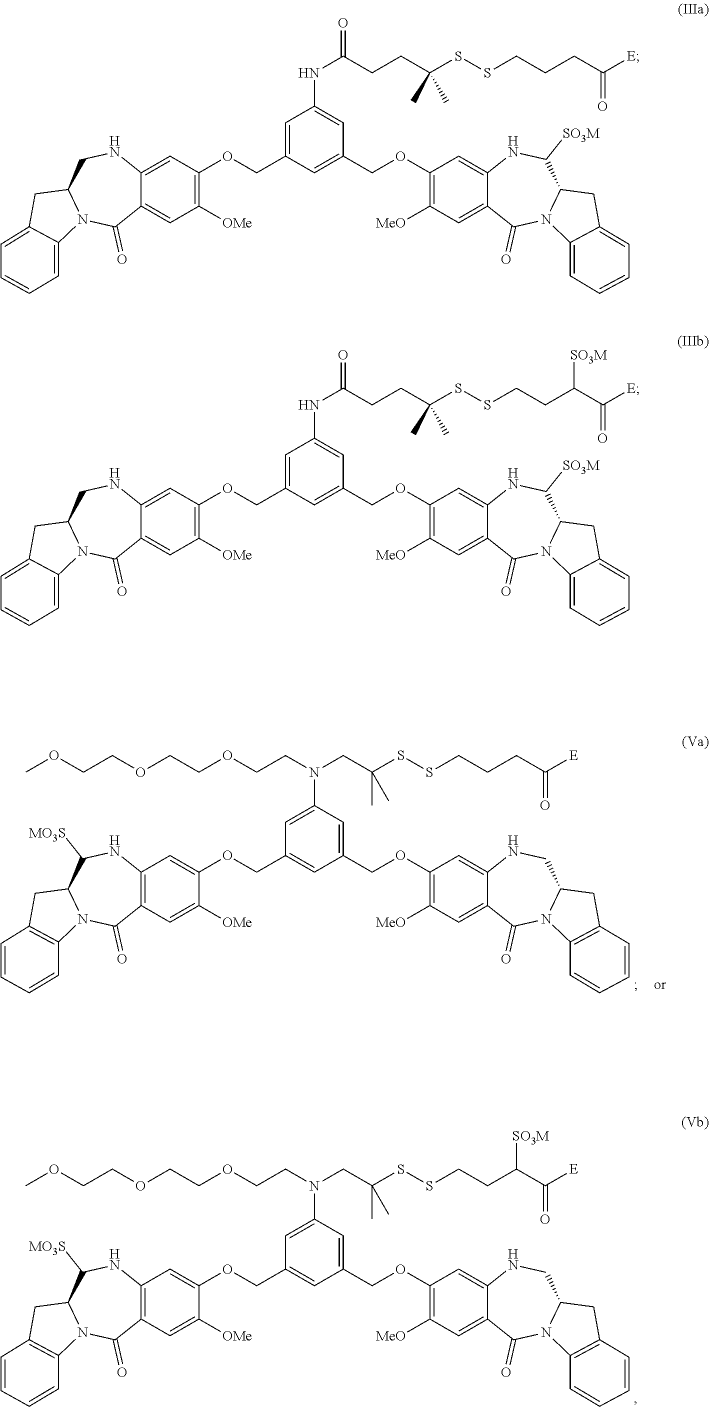

or a pharmaceutically acceptable salt thereof.

In a 41^{st} specific embodiment, for methods of present invention described herein (e.g., the method described in the first, second or third embodiment or the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th} or 30^{th} specific embodiment), the cytotoxic agent or the cytotoxic agent-linker compound is represented by one of the following formulas:

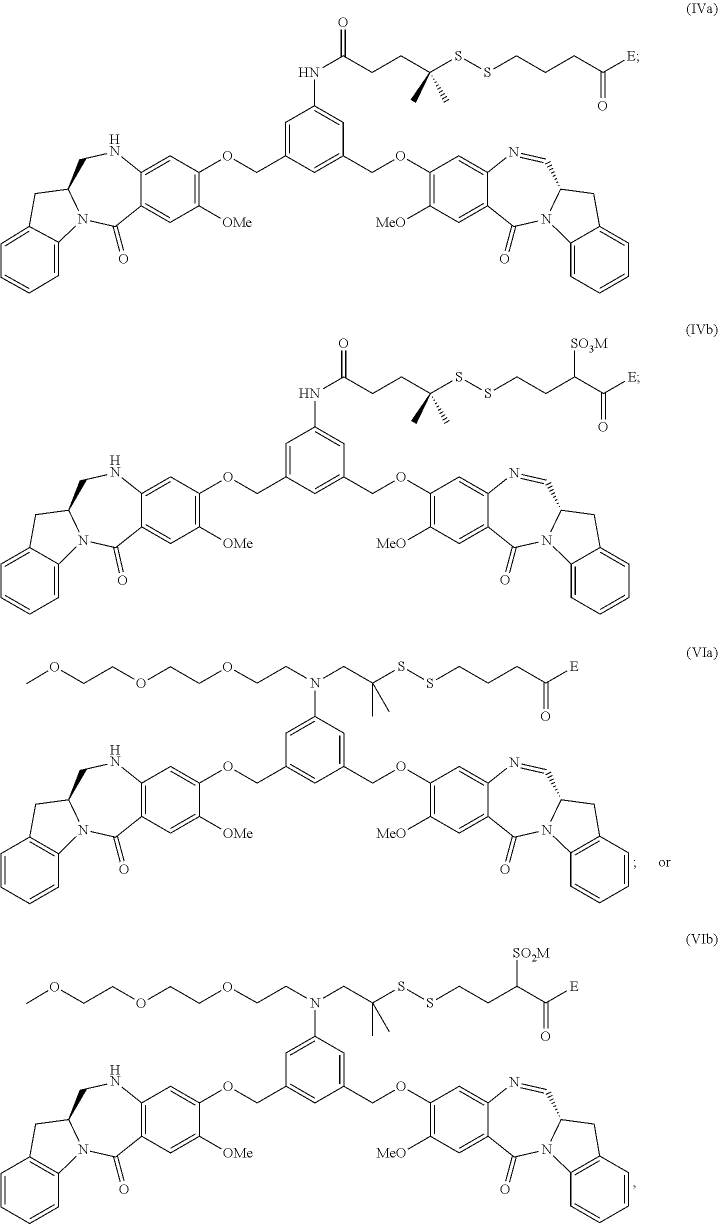

or a pharmaceutically acceptable salt thereof.

In a 42^{nd} specific embodiment, for methods of present invention described herein (e.g., the method described in the first, second or third embodiment or the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th} or 30^{th} specific embodiment), the cytotoxic agent or the cytotoxic agent-linker compound is represented by one of the following formulas:

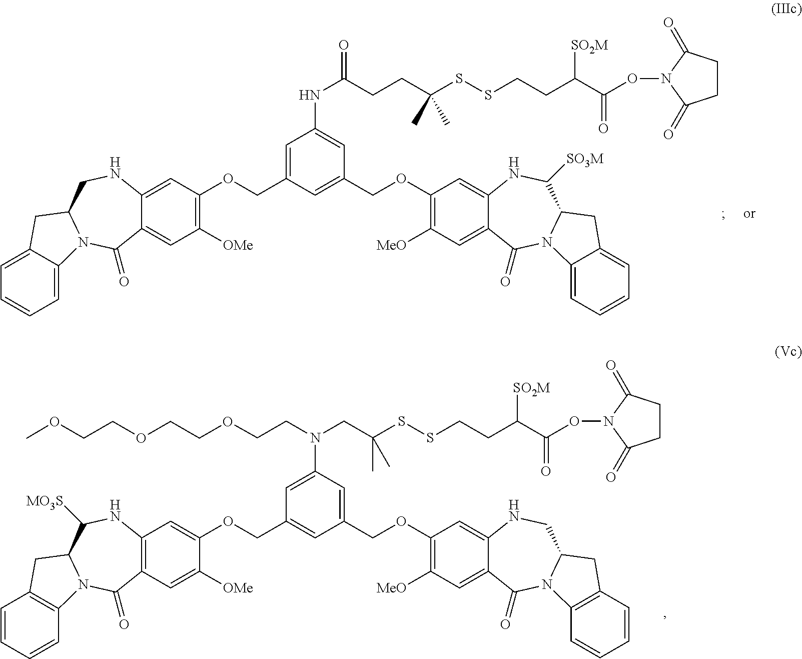

or a pharmaceutically acceptable salt thereof.

In a 43^{rd} specific embodiment, for methods of present invention described herein (e.g., the method described in the first, second or third embodiment or the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th} or 30^{th} specific embodiment), the cytotoxic agent or the cytotoxic agent-linker compound is represented by one of the following formulas:

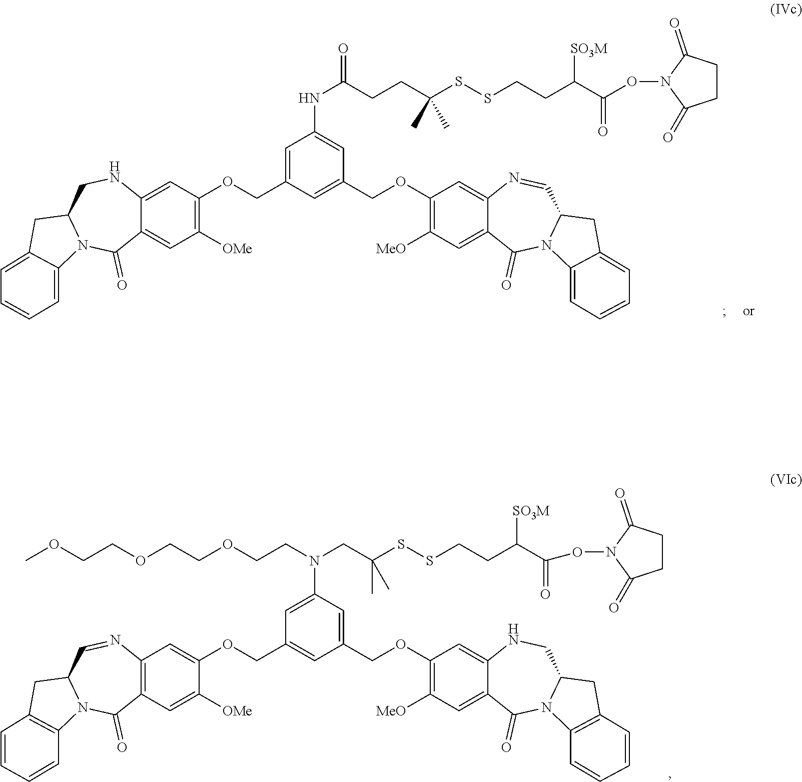

or a pharmaceutically acceptable salt thereof

In certain embodiments, the compounds represented by structural formula (I), (III) or (V) described above is prepared by reacting the compound of structural formula (II), (IV) or (VI) described above, respectively, with a sulfonating reagent.

As used herein, a “sulfonating reagent” is a reagent that can effect the following transformation.

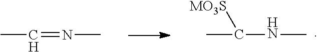

In one embodiment, the sulfonating reagent is NaHSO₃.

In certain embodiments, the compounds represented by structural formulas (Ia), (Ib), (Ic), (Id) or (Ie) are prepared by reacting the compound represented by structural formulas (IIa), (IIb), (IIc), (IId) and (IIe), respectively, with a sulfonating reagent.

In certain embodiments, the compounds represented by structural formulas (IIIa), (IIIb) or (IIIc) are prepared by reacting the compound represented by structural formulas (IVa), (IVb) or (IVc), respectively, with a sulfonating reagent.

In certain embodiments, the compounds represented by structural formulas (Va), (Vb) or (Vc) are prepared by reacting the compound represented by structural formulas (VIa), (VIb) or (VIc), respectively, with a sulfonating reagent.

In certain embodiments, for methods of the present invention described above (e.g., the method described in the first, second or the third embodiment or the 1^{st}, 2^{nd}, 3^{rd}, 4^{th}, 5^{th}, 6^{th}, 7^{th}, 8^{th}, 9^{th}, 10^{th}, 11^{th}, 13^{th}, 15^{th}, 16^{th}, 17^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 37^{th}, 38^{th}, 39^{th}, 40^{th}, 41^{st}, or 43^{rd} specific embodiment), the cytotoxic agent or the cytotoxic agent-linker compound is represented by structural formula (II), (IIa), (IIb), (IIc), (IId), (IIe), (IV), (IVa), (IVb), (IVc), (VIa), (VIb) or (VIc), and the method further comprises reacting the cell-binding agent-cytotoxic agent conjugate with a sulfonating reagent. In one embodiment, the sulfonating reagent is NaHSO₃ or KHSO₃.

In certain embodiments, for methods of the present invention described above (e.g., the method described in the first, second or third embodiment or the 1^{st}, 2^{nd}, 3^{rd}, 4^{th}, 5^{th}, 6^{th}, 7^{th}, 8^{th}, 9^{th}, 10^{th}, 11^{th}, 13^{th}, 15^{th}, 16^{th}, 17^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 37^{th}, 38^{th}, 39^{th}, 40^{th}, 41^{st}, or 43^{rd} specific embodiment), the cytotoxic agent or the cytotoxic agent-linker compound is represented by structural formula (II), (IIa), (IIb), (IIc), (IId), (lie), (IV), (IVa), (IVb), (IVc), (VIa), (VIb) or (VIc), the method comprises reacting the cell-binding agent with the cytotoxic agent or the cytotoxic agent-linker compound represented by structural formula (II), (IIa), (IIb), (IIc), (IId), (lie), (IV), (IVa), (IVb), (IVc), (VIa), (VIb) or (VIc), in the presence of a sulfonating reagent. In one embodiment, the sulfonating reagent is NaHSO₃ or KHSO₃.

In certain embodiments, the compounds represented by structural formula (IIIa), (Mb), (Va) or (Vb) are prepared by reacting a compound represented by one of the following structural formulas:

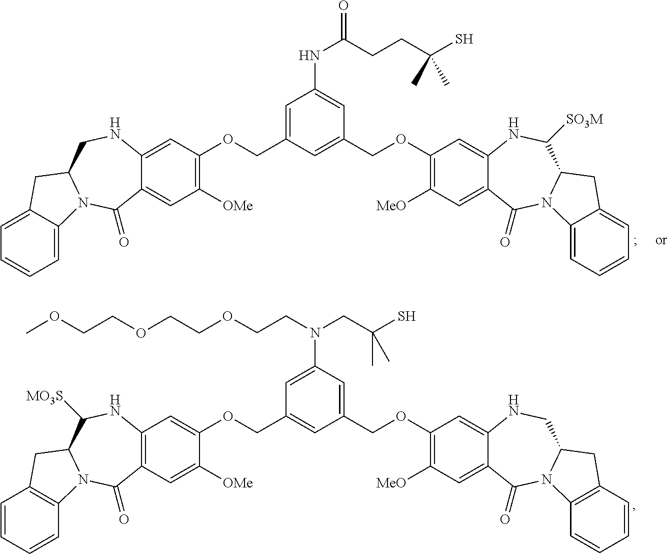

or a pharmaceutically acceptable salt thereof, with a linker compound represented by one of the following structural formulas:

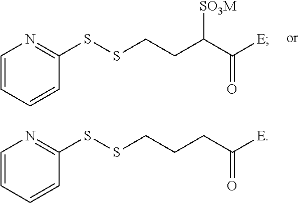

In certain embodiment, the compound of structural formula (IIIc) or (Vc) is prepared by reacting a compound represented by the following structural formula:

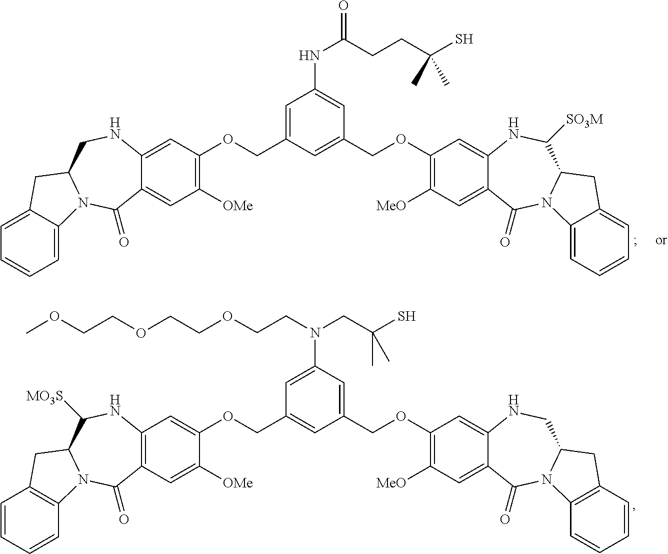

or a pharmaceutically acceptable salt thereof, with a linker compound of the following structural formula:

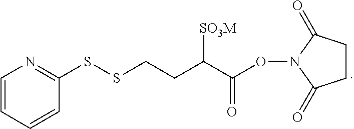

In one embodiment, for compounds described herein (e.g., compounds of formula (I), (Ia), (Ib), (Ic), (Id), (Ie), (II), (IIa), (IIb), (IIc), (IId), (IIe), (III), (IIIa), (IIIb), (IIIc), (IV), (IVa), (IVb), (IVc), (V), (Va), (Vb), (Vc), (VI), (VIa), (VIb), or (VIc)), M is —H, Na⁺ or K. In one embodiment, M is Na⁺ or K⁺. In another embodiment, M is Na⁺. In yet another embodiment, M is K⁺.

Other suitable cytotoxic agents include, for example, maytansinoids and conjugatable ansamitocins (see, for example, International Patent Application No. PCT/US11/59131, filed Nov. 3, 2011 and U.S. Pat. No. 9,090,629), taxoids, CC-1065 and CC-1065 analogs, and dolastatin and dolastatin analogs. In a specific embodiment of the invention, the cytotoxic agent is a maytansinoid, including maytansinol and maytansinol analogs. Maytansinoids are compounds that inhibit microtubule formation and are highly toxic to mammalian cells. Examples of suitable maytansinol analogues include those having a modified aromatic ring and those having modifications at other positions. Such maytansinoids are described in, for example, U.S. Pat. Nos. 4,256,746, 4,294,757, 4,307,016, 4,313,946, 4,315,929, 4,322,348, 4,331,598, 4,361,650, 4,362,663, 4,364,866, 4,424,219, 4,371,533, 4,450,254, 5,475,092, 5,585,499, 5,846,545, and 6,333,410.

Examples of maytansinol analogs having a modified aromatic ring include: (1) C-19-dechloro (U.S. Pat. No. 4,256,746) (prepared by LAH reduction of ansamytocin P2), (2) C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH), and (3) C-20-demethoxy, C-20-acyloxy (—OCOR), +/−dechloro (U.S. Pat. No. 4,294,757) (prepared by acylation using acyl chlorides).

Examples of maytansinol analogs having modifications of positions other than an aromatic ring include: (1) C-9-SH (U.S. Pat. No. 4,424,219) (prepared by the reaction of maytansinol with H.sub.2S or P.sub.2S.sub.5), (2) C-14-alkoxymethyl (demethoxy/CH.sub.20R) (U.S. Pat. No. 4,331,598), (3) C-14-hydroxymethyl or acyloxymethyl (CH.sub.20H or CH.sub.2OAc) (U.S. Pat. No. 4,450,254) (prepared from Nocardia), (4) C-15-hydroxy/acyloxy (U.S. Pat. No. 4,364,866) (prepared by the conversion of maytansinol by Streptomyces), (5) C-15-methoxy (U.S. Pat. Nos. 4,313,946 and 4,315,929) (isolated from Trewia nudiflora), (6) C-18-N-demethyl (U.S. Pat. Nos. 4,362,663 and 4,322,348) (prepared by the demethylation of maytansinol by Streptomyces), and (7) 4,5-deoxy (U.S. Pat. No. 4,371,533) (prepared by the titanium trichloride/LAH reduction of maytansinol).

In a specific embodiment of the invention, the cytotoxic agent can be used in the processes of the present invention is the thiol-containing maytansinoid DM1, also known as N^{2′}-deacetyl-N^{1′}-(3-mercapto-1-oxopropyl)-maytansine. The structure of DM1 is shown below:

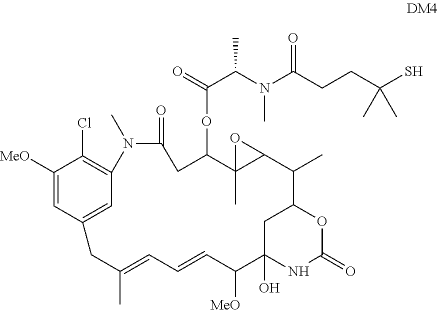 [SMILES: COc1cc2cc(c1Cl)N(C)C(=O)CC(OC(=O)[C@H](C)N(C)C(=O)CCC(C)(C)S)C1(C)OC1C(C)C1CC(O)(NC(=O)O1)C(OC)/C=C/C=C(\C)C2]

In another specific embodiment of the invention, the cytotoxic agent can be used in the processes of the present invention is the thiol-containing maytansinoid DM1, also known as N^{2′}-deacetyl-N^{2′}-(4-methyl-4-mercapto-1-oxopentyl)-maytansine. The structure of DM4 shown below:

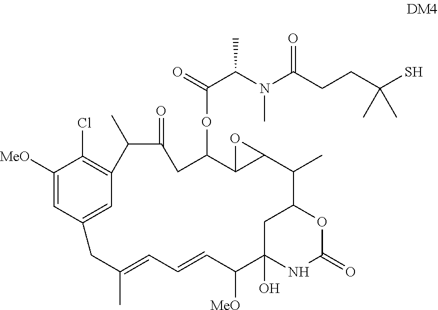 [SMILES: COc1cc2cc(c1Cl)C(C)C(=O)CC(OC(=O)[C@H](C)N(C)C(=O)CCC(C)(C)S)C1OC1C(C)C1CC(O)(NC(=O)O1)C(OC)/C=C/C=C(\C)C2]

Other maytansinoids may be used in the context of the invention, including, for example, thiol and disulfide-containing maytansinoids bearing a mono or di-alkyl substitution on the carbon atom bearing the sulfur atom. Particularly preferred is a maytansinoid having at the C-3 position (a) C-14 hydroxymethyl, C-15 hydroxy, or C-20 desmethyl functionality, and (b) an acylated amino acid side chain with an acyl group bearing a hindered sulfhydryl group, wherein the carbon atom of the acyl group bearing the thiol functionality has one or two substituents, said substituents being a linear or branched alkyl or alkenyl having from 1 to 10 carbon atoms, cyclic alkyl or alkenyl having from 3 to 10 carbon atoms, phenyl, substituted phenyl, or heterocyclic aromatic or heterocycloalkyl radical, and further wherein one of the substituents can be H, and wherein the acyl group has a linear chain length of at least three carbon atoms between the carbonyl functionality and the sulfur atom.

In order that the invention described herein may be more fully understood, the following examples are set forth. It should be understood that these examples are for illustrative purposes only and are not to be construed as limiting this invention in any manner.

Also included in the present invention is the cell-binding agent-cytotoxic agent conjugates prepared by any methods described herein (e.g., method described in the first, second or third embodiment or the 1^{st}, 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd} specific embodiment).

In one embodiment, the conjugates prepared by methods of the present invention is represented by one of the following structural formulas:

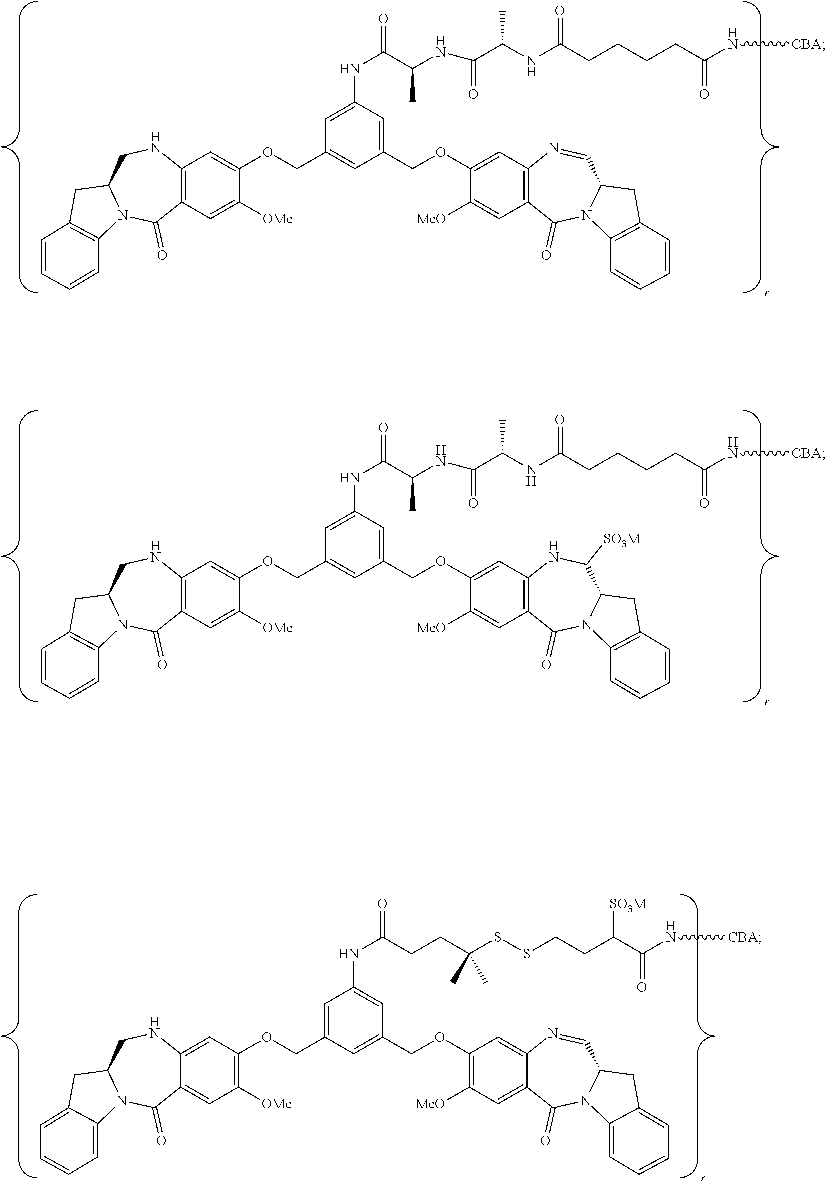

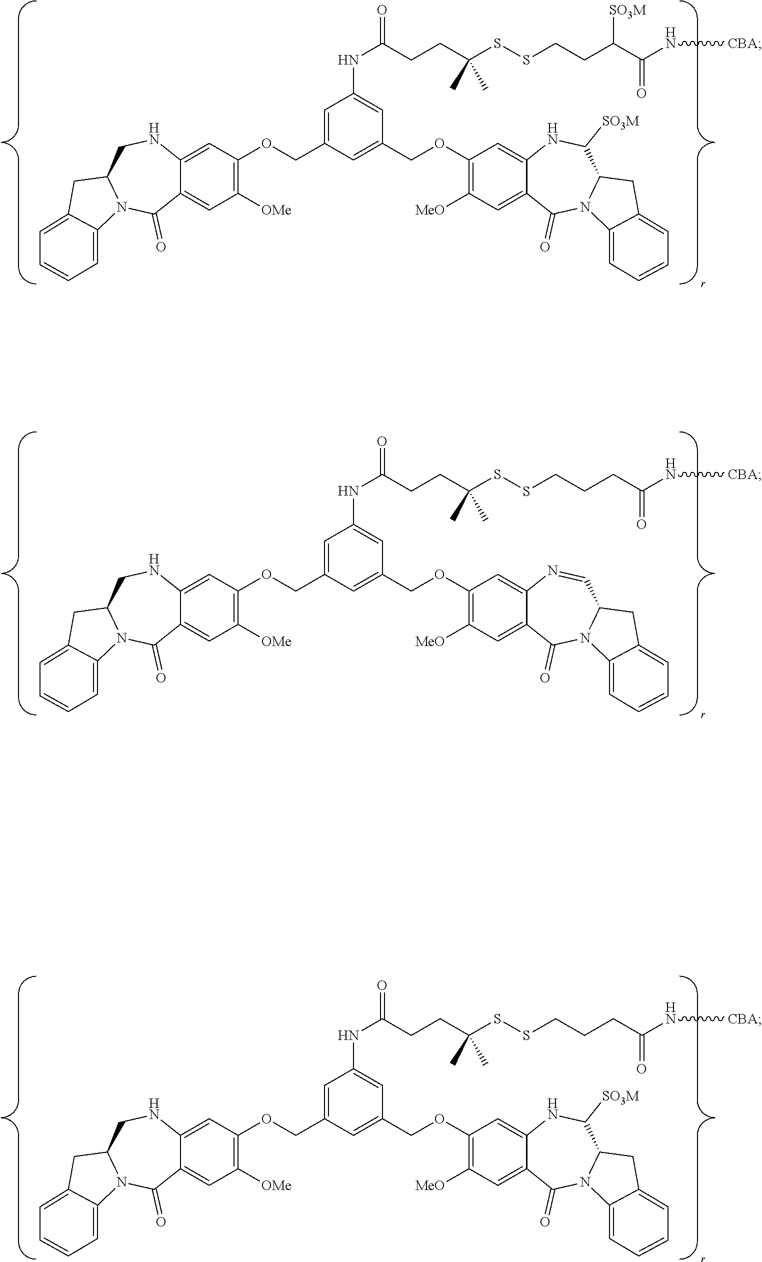

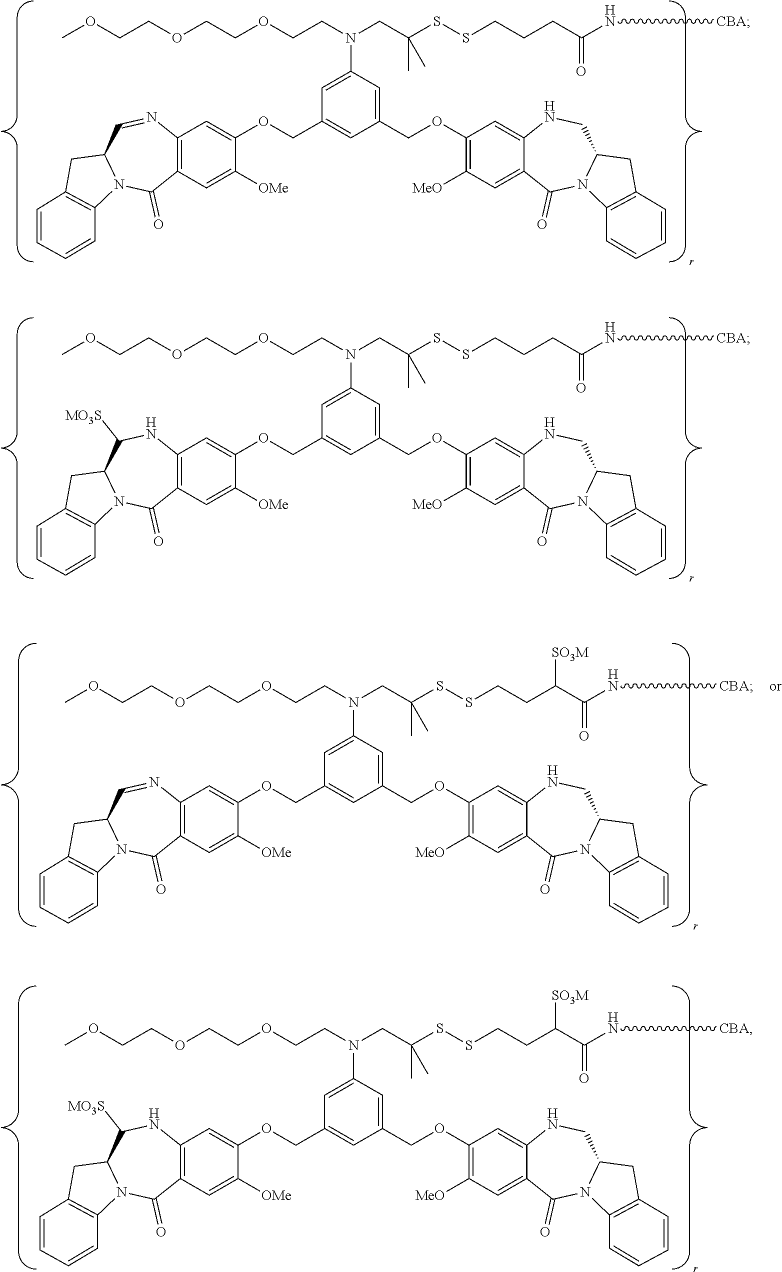

or a pharmaceutically acceptable salt thereof, wherein CBA-NH₂ is the cell-binding agent; M is —H or a pharmaceutically acceptable cation, such as Na⁺ or K⁺; and r is an integer from 1 to 10.

### EXAMPLES

### Example 1

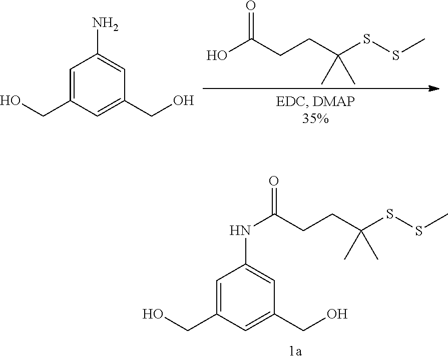 [SMILES: Nc1cc(CO)cc(CO)c1 ; CSSC(C)(C)CCC(=O)O ; CSSC(C)(C)CCC(=O)Nc1cc(CO)cc(CO)c1]

### Compound 1a:

To a stirred solution of (5-amino-1,3-phenylene)dimethanol (1.01 g, 6.59 mmol) in anhydrous dimethylformamide (16.48 mL) and anhydrous tetrahydrofuran (16.48 ml) was added 4-methyl-4-(methyldisulfanyl)pentanoic acid (1.281 g, 6.59 mmol), N-β-dimethylaminopropyl)-N′-ethylcarbodiimide hydrochloride (2.53 g, 13.19 mmol), and 4-dimethylaminopyridine (0.081 g, 0.659 mmol). The resulting mixture was stirred for 18 hours at room temperature. The reaction was quenched with saturated ammonium chloride solution and extracted with ethyl acetate (3×50 mL). The organic extracts were washed with water and brine, then dried over anhydrous sodium sulfate. The solution was filtered and concentrated in vacuo and the resulting residue was purified by silica gel chromatography (Ethyl acetate/Hexanes) to obtain compound 1a as a white solid (0.70 g, 32% yield). ¹H NMR (400 MHz, DMSO-d6: δ 9.90 (s, 1H), 7.43 (s, 2H), 6.93 (s, 1H), 5.16 (t, 2H, J=5.7 Hz), 4.44 (d, 4H, J=5.7 Hz), 2.43 (s, 3H), 2.41-2.38 (m, 2H), 1.92-1.88 (m, 2H), 1.29 (s, 6H). MS (m/z), found 330.0 (M+1)⁺.

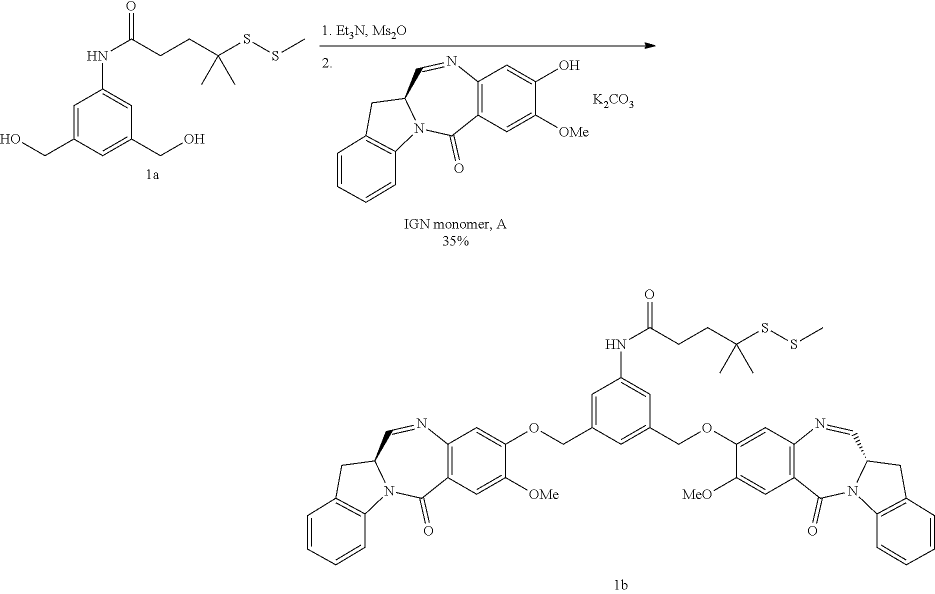

### Compound 1b:

To a cooled (−10° C.) solution of compound 1a (219 mg, 0.665 mmol) in anhydrous dichloromethane (6.65 mL) was added triethylamine (463 μl, 3.32 mmol) followed by dropwise addition of methanesulfonic anhydride (298 mg, 1.662 mmol). The mixture stirred at −10° C. for 2 hours, then the mixture was quenched with ice water and extracted with cold ethyl acetate (2×30 mL). The organic extracts were washed with ice water, dried with anhydrous sodium sulfate, filtered and concentrated under reduced pressure to obtain the crude dimesylate.

The crude dimesylate (227 mg, 0.467 mmol) and IGN monomer A (303 mg, 1.028 mmol) were dissolved in anhydrous DMF (3.11 mL). Potassium carbonate (161 mg, 1.169 mmol) was added and the mixture stirred for 18 hours at room temperature. Deionized water was added and the resulting precipitate was filtered and rinsed with water. The solid was re-dissolved in dichloromethane and washed with water. The organic layer was dried with anhydrous magnesium sulfate, filtered, and concentrated. The crude residue was purified by silica gel chromatography (Methanol/Dichloromethane) to give compound 1b (227 mg, 36% yield). MS (m/z), found 882.5 (M+1)⁺.

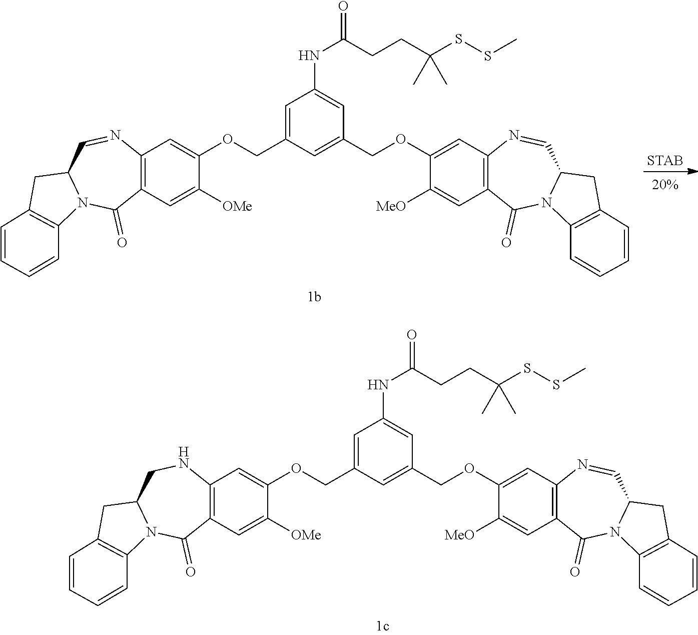 [SMILES: COc1cc2c(cc1OCc1cc(COc3cc4c(cc3OC)C(=O)N3c5ccccc5C[C@H]3C=N4)cc(NC(=O)CCC(C)(C)SSC)c1)N=C[C@@H]1Cc3ccccc3N1C2=O ; COc1cc2c(cc1OCc1cc(COc3cc4c(cc3OC)C(=O)N3c5ccccc5C[C@H]3CN4)cc(NC(=O)CCC(C)(C)SSC)c1)N=C[C@@H]1Cc3ccccc3N1C2=O]

### Compound 1c:

To a suspension of compound 1b (227 mg, 0.167 mmol) in anhydrous 1,2-dichloroethane (3.346 mL) was added sodium triacetoxyborohydride (37.3 mg, 0.167 mmol). The mixture was stirred at room temp for one hour upon which it was quenched with saturated ammonium chloride solution. The mixture was extracted with dichloromethane and washed with brine. The organic layer was dried with anhydrous magnesium sulfate, filtered and concentrated. The crude residue was purified by RP-HPLC (C18, Water/Acetonitrile). Fractions containing desired product were extracted with dichloromethane, dried with anhydrous magnesium sulfate, filtered and concentrated to give compound 1c (35 mg, 19% yield). MS (m/z), found 884.3 (M+1)⁺.

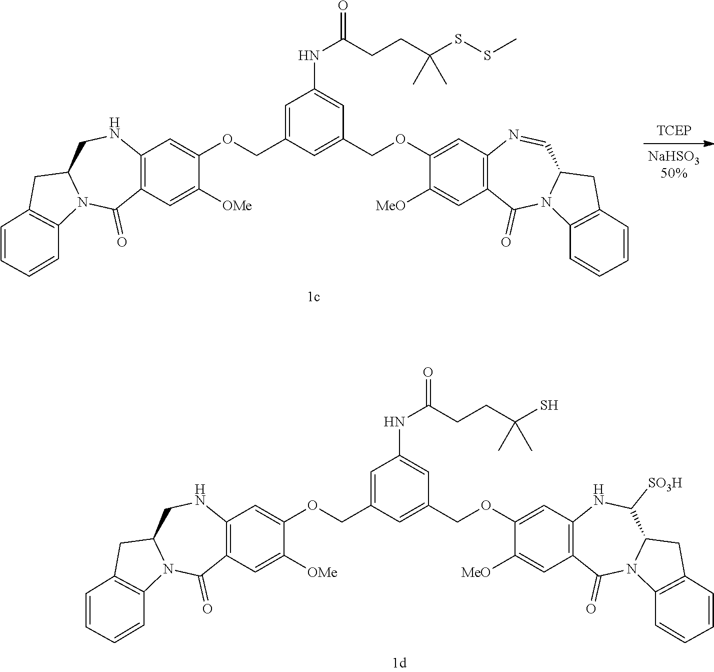

### Compound 1d:

To a solution of compound 1c (18 mg, 0.017 mmol) in acetonitrile (921 μL) and methanol (658 μL) was added tris(2-carboxyethyl)phosphine hydrochloride (17.51 mg, 0.060 mmol) (neutralized with saturated sodium bicarbonate solution (0.2 mL) in sodium phosphate buffer (132 μL, 0.75 M, pH 6.5). The mixture was stirred at room temperature for 3.5 hours, then diluted with dichloromethane and deionized water. The organic layer was separated, washed with brine, dried with anhydrous sodium sulfate, filtered and concentrated under reduced pressure to obtain the crude thiol. MS (m/z), found 838.3 (M+1)⁺.

The crude thiol from step 5 (15.5 mg, 0.018 mmol) was dissolved in 2-propanol (1.23 mL). Deionized water (617 μL) and sodium bisulfite (5.77 mg, 0.055 mmol) were added and the mixture stirred for five hours at room temperature. The reaction was frozen in an acetone/dry ice bath, lyophilized, and purified by RP-HPLC (C18, deionized water/acetonitrile). Fractions containing desired product were frozen and lyophilized to give compound (12S,12aS)-9-(β-(4-mercapto-4-methylpentanamido)-5-((((R)-8-methoxy-6-oxo-11,12,12a,13-tetrahydro-6H-benzo[5,6][1,4]diazepino[1,2-a]indol-9-yl)oxy)methyl)benzyl)oxy)-8-methoxy-6-oxo-11,12,12a,13-tetrahydro-6H-benzo[5,6][1,4]diazepino[1,2-a]indole-12-sulfonic acid (compound 1d) (6.6 mg, 39% yield). MS (m/z), found 918.2 (M−1)⁻.

### Example 2

Synthesis of 2,5-dioxopyrrolidin-1-yl 6-(((S)-1-(((S)-1-((3-((((S)-8-methoxy-6-oxo-11,12,12a,13-tetrahydro-6H-benzo[5,6][1,4]diazepino[1,2-a]indol-9-yl)oxy)methyl)-5-((((R)-8-methoxy-6-oxo-12a,13-dihydro-6H-benzo[5,6][1,4]diazepino[1,2-a]indol-9-yl)oxy)methyl) phenyl)amino)-1-oxopropan-2-yl)amino)-1-oxopropan-2-yl)amino)-6-oxohexanoate, compound 90.

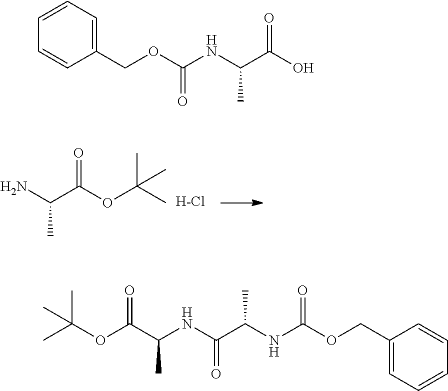

Step 1: (S)-2-(((benzyloxy)carbonyl)amino)propanoic acid (5 g, 22.40 mmol) and (S)-tert-butyl 2-aminopropanoate hydrochloride (4.48 g, 24.64 mmol) were dissolved in anhydrous DMF (44.8 mL). EDC.HCl (4.72 g, 24.64 mmol), HOBt (3.43 g, 22.40 mmol), and DIPEA (9.75 mL, 56.0 mmol) were added. The reaction stirred under argon, at room temperature, overnight. The reaction mixture was diluted with dichloromethane and then washed with saturated ammonium chloride, saturated sodium bicarbonate, water, and brine. The organic layer was dried over sodium sulfate and concentrated. The crude oil was purified via silica gel chromatography (Hexanes/Ethyl Acetate) to yield compound 2a (6.7 g, 85% yield). ¹H NMR (400 MHz, CDCl₃): δ 7.38-7.31 (m, 5H), 6.53-6.42 (m, 1H), 5.42-5.33 (m, 1H), 5.14 (s, 2H), 4.48-4.41 (m, 1H), 4.32-4.20 (m, 1H), 1.49 (s, 9H), 1.42 (d, 3H, J=6.8 Hz), 1.38 (d, 3H, J=7.2 Hz).

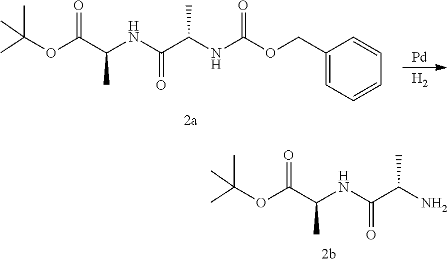

Step 2: Compound 2a (6.7 g, 19.12 mmol) was dissolved in methanol (60.7 mL) and water (3.03 mL). The solution was purged with argon for five minutes. Palladium on carbon (wet, 10%) (1.017 g, 0.956 mmol) was added slowly. The reaction was stirred overnight under an atmosphere of hydrogen. The solution was filtered through Celite, rinsed with methanol and concentrated. It was azeotroped with methanol and acetonitrile and the resulting oil was placed directly on the high vacuum to give compound 2b (4.02 g, 97% yield) which was used directly in the next step. ¹H NMR (400 MHz, CDCl₃): δ 7.78-7.63 (m, 1H), 4.49-4.42 (m, 1H), 3.55-3.50 (m, 1H), 1.73 (s, 2H), 1.48 (s, 9H), 1.39 (d, 3H, J=7.2 Hz), 1.36 (d, 3H, J=6.8 Hz).

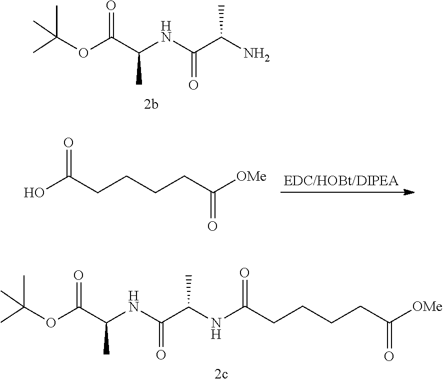 [SMILES: C[C@H](N)C(=O)N[C@@H](C)C(=O)OC(C)(C)C ; COC(=O)CCCCC(=O)O ; COC(=O)CCCCC(=O)N[C@@H](C)C(=O)N[C@@H](C)C(=O)OC(C)(C)C]

Step 3: Compound 2b (4.02 g, 18.59 mmol) and mono methyladipate (3.03 mL, 20.45 mmol) were dissolved in anhydrous DMF (62.0 mL). EDC.HCl (3.92 g, 20.45 mmol), HOBt (2.85 g, 18.59 mmol) and DIPEA (6.49 mL, 37.2 mmol) were added. The mixture was stirred overnight at room temperature. The reaction was diluted with dichloromethane/methanol (150 mL, 5:1) and washed with saturated ammonium chloride, saturated sodium bicarbonate, and brine. It was dried over sodium sulfate, filtered and stripped. The compound was azeotroped with acetonitrile (5×), then pumped on the high vacuum at 35° C. to give compound 2c (6.66 g, 100% yield). The crude material was taken onto next step without purification. ¹H NMR (400 MHz, CDCl₃): δ 6.75 (d, 1H, J=6.8 Hz), 6.44 (d, 1H, J=6.8 Hz), 4.52-4.44 (m, 1H), 4.43-4.36 (m, 1H), 3.65 (s, 3H), 2.35-2.29 (m, 2H), 2.25-2.18 (m, 2H), 1.71-1.60 (m, 4H), 1.45 (s, 9H), 1.36 (t, 6H, J=6.0 Hz).

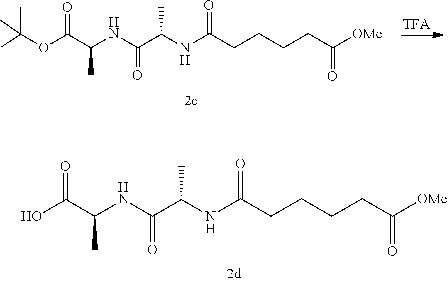 [SMILES: COC(=O)CCCCC(=O)N[C@@H](C)C(=O)N[C@@H](C)C(=O)OC(C)(C)C ; COC(=O)CCCCC(=O)N[C@@H](C)C(=O)N[C@@H](C)C(=O)O]

Step 4: Compound 2c (5.91 g, 16.5 mmol) was stirred in TFA (28.6 mL, 372 mmol) and deionized water (1.5 mL) at room temperature for three hours. The reaction mixture was concentrated with acetonitrile and placed on high vacuum to give crude compound 2d as a sticky solid (5.88 g, 100% yield). ¹H NMR (400 MHz, CDCl₃): δ 7.21 (d, 1H, J=6.8 Hz), 6.81 (d, 1H, J=7.6 Hz), 4.69-4.60 (m, 1H), 4.59-4.51 (m, 1H), 3.69 (s, 3H), 2.40-2.33 (m, 2H), 2.31-2.24 (m, 2H), 1.72-1.63 (m, 4H), 1.51-1.45 (m, 3H), 1.42-1.37 (m, 3H).

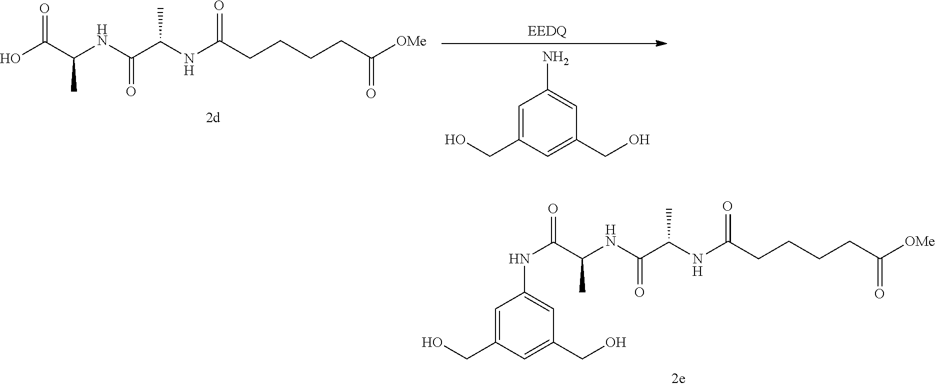 [SMILES: COC(=O)CCCCC(=O)N[C@@H](C)C(=O)N[C@@H](C)C(=O)O ; Nc1cc(CO)cc(CO)c1 ; COC(=O)CCCCC(=O)N[C@@H](C)C(=O)N[C@@H](C)C(=O)Nc1cc(CO)cc(CO)c1]

Step 5: Compound 2d (5.6 g, 18.52 mmol) was dissolved in anhydrous dichloromethane (118 mL) and anhydrous methanol (58.8 mL). (5-amino-1,3-phenylene)dimethanol (2.70 g, 17.64 mmol) and EEDQ (8.72 g, 35.3 mmol) were added and the reaction was stirred at room temperature, overnight. The solvent was stripped and ethyl acetate was added. The resulting slurry was filtered, washed with ethyl acetate and dried under vacuum/N₂ to give compound 2e (2.79 g, 36% yield). ¹H NMR (400 MHz, DMSO-d6): δ 9.82 (s, 1H), 8.05, (d, 1H, J=9.2 Hz), 8.01 (d, 1H, J=7.2 Hz), 7.46 (s, 2H), 6.95 (3, 1H), 5.21-5.12 (m, 2H), 4.47-4.42 (m, 4H), 4.40-4.33 (m, 1H), 4.33-4.24 (m, 1H), 3.58 (s, 3H), 2.33-2.26 (m, 2H), 2.16-2.09 (m, 2H), 1.54-1.46 (m, 4H), 1.30 (d, 3H, J=7.2 Hz), 1.22 (d, 3H, J=4.4 Hz).

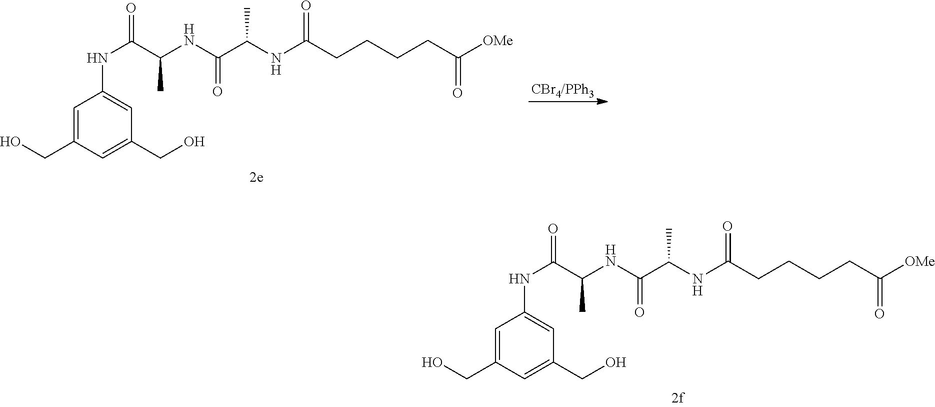 [SMILES: COC(=O)CCCCC(=O)N[C@@H](C)C(=O)N[C@@H](C)C(=O)Nc1cc(CO)cc(CO)c1 ; COC(=O)CCCCC(=O)N[C@@H](C)C(=O)N[C@@H](C)C(=O)Nc1cc(CO)cc(CO)c1]

Step 6: Compound 2e (0.52 g, 1.189 mmol) and carbon tetrabromide (1.183 g, 3.57 mmol) were dissolved in anhydrous DMF (11.89 mL). Triphenylphosphine (0.935 g, 3.57 mmol) was added and the reaction stirred under argon for four hours. The reaction mixture was diluted with DCM/MeOH (10:1) and washed with water and brine, dried over sodium sulfate, filtered, and concentrated. The crude material was purified by silica gel chromatography (DCM/MeOH) to give compound 2f (262 mg, 39% yield). ¹H NMR (400 MHz, DMSO-d6): δ 10.01 (s, 1H), 8.11 (d, 1H, J=6.8 Hz), 8.03 (d, 1H, J=6.8 Hz), 7.67 (s, 2H), 7.21 (s, 1H), 4.70-4.64 (m, 4H), 4.40-4.32 (m, 1H), 4.31-4.23 (m, 1H), 3.58 (s, 3H), 2.34-2.26 (m, 2H), 2.18-2.10 (m, 2H) 1.55-1.45 (m, 4H) 1.31 (d, 3H, J==7.2 Hz), 1.21 (d, 3H, J=7.2 Hz).

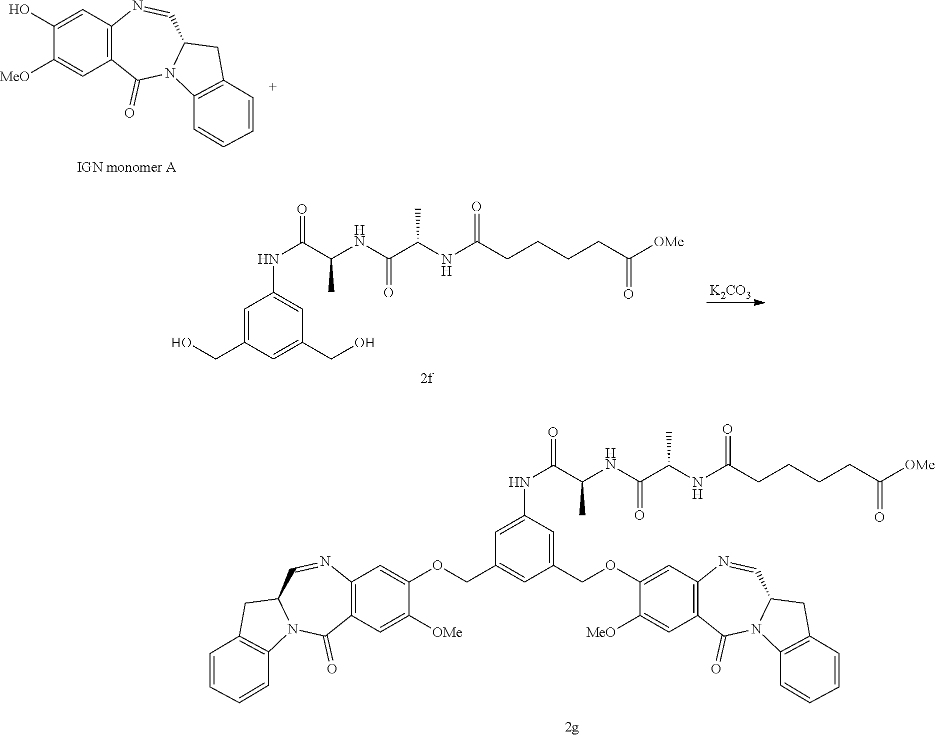

Step 7: Dibromide compound 2f and IGN monomer compound A were dissolved in DMF. Potassium carbonate was added and was stirred at room temperature overnight. Water was added to the reaction mixture to precipitate the product. The slurry was stirred at room temperature and was then filtered and dried under vacuum/N₂. The crude material was purified by silica gel chromatography (dichloromethane/methanol) to give compound 2g (336 mg, 74% yield). LCMS=5.91 min (15 min method). MS (m/z): 990.6 (M+1)⁺.

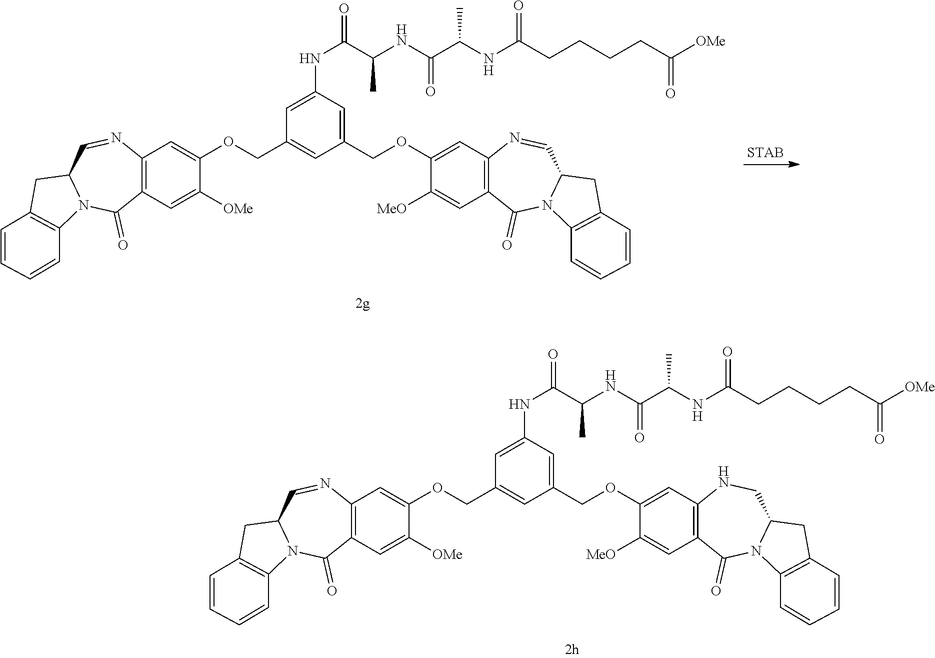 [SMILES: COC(=O)CCCCC(=O)N[C@@H](C)C(=O)N[C@@H](C)C(=O)Nc1cc(COc2cc3c(cc2OC)C(=O)N2c4ccccc4C[C@H]2C=N3)cc(COc2cc3c(cc2OC)C(=O)N2c4ccccc4C[C@H]2C=N3)c1 ; COC(=O)CCCCC(=O)N[C@@H](C)C(=O)N[C@@H](C)C(=O)Nc1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

Step 8: Diimine compound 2g was dissolved in 1,2-dichloroethane. NaBH(OAc)₃ (STAB) was added to the reaction mixture and was stirred at room temperature for 1 h. The reaction was diluted with CH₂Cl₂ and was quenched with saturated NH₄Cl solution. The layers were separated and was washed with brine, dried over Na₂SO₄ and concentrated. The crude material was purified via RPHPLC (C18 column, Acetonitrile/Water) to give compound 2h (85.5 mg, 25% yield). LCMS=6.64 min (15 min method). MS (m/z): 992.6 (M+1)⁺.

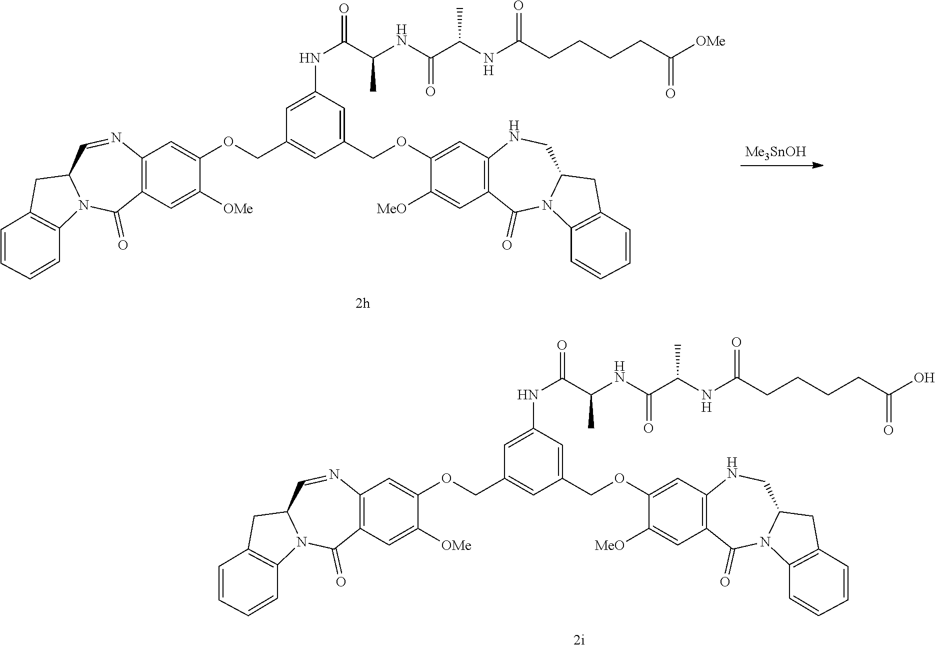 [SMILES: "Me3SnOH" ; COC(=O)CCCCC(=O)N[C@@H](C)C(=O)N[C@@H](C)C(=O)Nc1cc(COc2cc3c(cc2OC)C(=O)N2c4ccccc4C[C@H]2C=N3)cc(COc2cc3c(cc2OC)C(=O)N2c4ccccc4C[C@H]2CN3)c1 ; COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)O)c1)N=C[C@@H]1Cc3ccccc3N1C2=O]

Step 9: Compound 2h was dissolved in 1,2-dichloroethane. Trimethylstannanol was added to the reaction mixture and was heated at 80° C. overnight. The reaction mixture was then cooled to RT and diluted with water. The aqueous layer was acidified to pH ˜4 with 1 M HCl. The mixture was extracted with CH₂Cl₂/MeOH. The combined organic layers were washed with brine, dried over Na₂SO₄, and concentrated. The crude material was passed through a silica plug to give compound 2i (48.8 mg, 80% yield). LCMS=5.89 min (15 min method). MS (m/z): 978.6 (M+1)⁺.

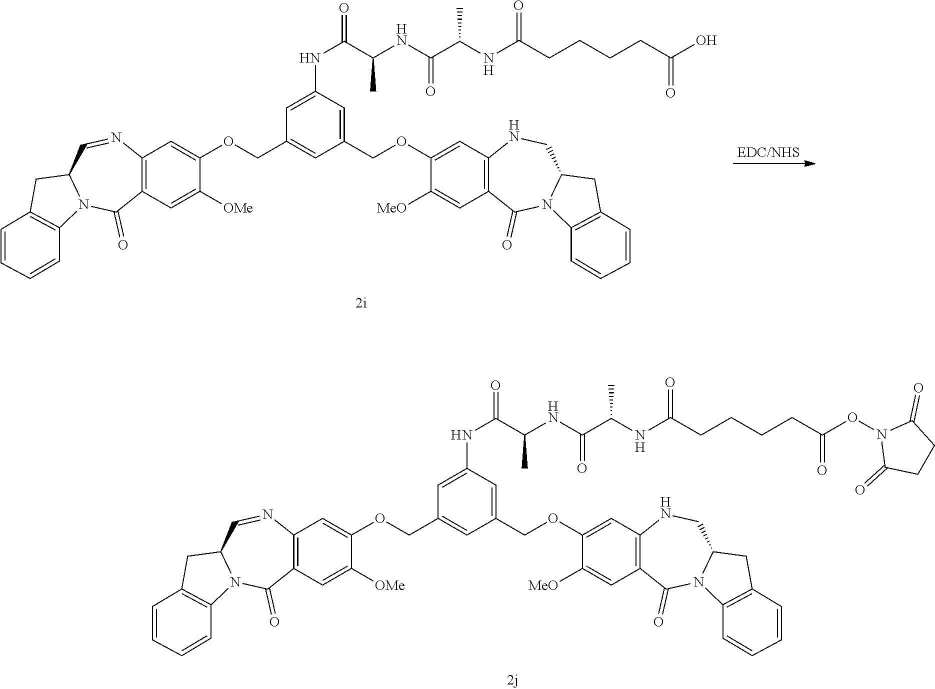 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)O)c1)N=C[C@@H]1Cc3ccccc3N1C2=O ; COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)ON3C(=O)CCC3=O)c1)N=C[C@@H]1Cc3ccccc3N1C2=O]

Step 10: EDC.HCl was added to a stirred solution of acid compound 2i and N-hydroxysuccinamide in CH₂Cl₂ at RT. The reaction mixture was stirred for 2 hrs. The reaction mixture was diluted with CH₂Cl₂ and washed with water and brine. The organic layer was dried over Na₂SO₄, filtered, and concentrated. The crude material was purified via RPHPLC (C18 column, Acetonitrile/Water) to give 2,5-dioxopyrrolidin-1-yl 6-(((S)-1-(((S)-1-((3-((((S)-8-methoxy-6-oxo-11,12,12a,13-tetrahydro-6H-benzo[5,6][1,4]diazepino[1,2-a]indol-9-yl)oxy)methyl)-5-((((R)-8-methoxy-6-oxo-12a,13-dihydro-6H-benzo[5,6][1,4]diazepino[1,2-a]indol-9-yl)oxy)methyl) phenyl)amino)-1-oxopropan-2-yl)amino)-1-oxopropan-2-yl)amino)-6-oxohexanoate, compound 2j (8.2 mg, 30% yield). LCMS=6.64 min (15 min method). MS (m/z): 1075.4 (M+1)⁺.

### Example 3

### Conjugation: Prior Protocol

AbX, a human anti-GCC antibody, 5F9 (having a heavy chain amino acid sequence of SEQ ID NO:19 and a light chain amino acid sequence of SEQ ID NO:20) was buffer exchanged into 15 mM HEPES, pH 8.5 prior to conjugation. AbX-(Ie) conjugates were then prepared using sulfonated form of compound (IIe). Compound (Ie) was initially sulfonated through incubation of compound (IIe) with a 5-fold molar excess of sodium bisulfite and 50 mM succinate (pH 5.0) in a 90/10 organic:aqueous solution at ambient temperature for 3 hrs followed by overnight incubation at 4° C. The conjugation reaction was then performed using 2.0 mg/mL of AbX antibody in 15 mM HEPES, pH 8.5 and the addition of compound (Ie) at a specified molar excess based on the antibody (see Table 1 for representative conjugation). The conjugation reaction had a final 90/10 aqueous:organic composition of 15 mM HEPES, pH 8.5 and DMA, and was incubated in a water bath at 25° C. for 4 hrs prior to purification into formulation buffer (10 mM histidine, 50 mM sodium chloride, 8.5% sucrose, 0.01% Tween-20, 50 μM sodium bisulfite, pH 6.2).

TABLE 1

| | Molar | | |
|---|---|---|---|
| | Excess of | Conjugation | Conjugation |
| Conjugate | compound (Ie) | Scale (mg) | Yield |
| AbX-(Ie) | 5.4 | 220 | 24% |

### Conjugation: Optimized Protocol

Various parameters including isotonic strength, conductivity, pH, reaction concentration, and molar equivalents of compound (Ie) were explored to optimize the yield of desired AbX-(Ie) conjugate. An optimized protocol utilizing 75 mM EPPS, pH 8.0 buffer emerged from these studies. Similar to the standard platform protocol, AbX-(Ie) conjugates were made using compound (Ie), sulfonated form of compound (IIe) (prepared as described in the previous section). The optimized conjugation reaction was carried out using 2.0 mg/mL of AbX antibody in 75 mM EPPS, pH 8.0 and the addition of compound (Ie) at a specified molar excess based on the antibody (see Table 2 for representative conjugation). The conjugation reaction had a final 90/10 aqueous:organic composition of 75 mM EPPS, pH 8.0 and DMA, and was incubated in a water bath at 25° C. for 4 hrs prior to purification into formulation buffer (10 mM histidine, 50 mM sodium chloride, 8.5% sucrose, 0.01% Tween-20, 50 μM sodium bisulfite, pH 6.2).

TABLE 2

| | Molar | | |
|---|---|---|---|
| | Excess of | Conjugation | Conjugation |
| Conjugate | compound (Ie) | Scale (mg) | Yield |
| AbX-(Ie) | 4.0 | 60 | 64% |

As shown in Table 2, the conjugation yield increased 24% to 64%, ˜2 fold increase, with protocols involving the use of buffer with higher ionic strength at pH 8.0 as compared to prior protocol using buffer with lower ionic strength at pH 8.5.

### Purification

The AbX-(Ie) conjugation reaction mixture was purified using Sephadex G-25 NAP columns equilibrated with 20 mM histidine, 50 mM sodium chloride, 8.5% sucrose, 0.01% Tween-20, and 50 μM sodium bisulfite, pH 6.2. The purified conjugate was filtered using a 0.22 μm PVDF syringe filter and dialyzed overnight against fresh formulation buffer at 4° C., followed by dialysis at ambient temperature for 4 hrs using fresh formulation buffer. The conjugate was re-filtered using a 0.22 μm PVDF syringe filter before analysis.

### Analytical Methods:

The concentration of antibody and cytotoxic agent (D) in purified conjugate samples was determined by UV/Vis using absorbance values at 280 nm and 330 nm. Since both the antibody and the cytotoxic agent absorb at 280 nm, a binomial equation was required to consider the portion of total signal attributed to each moiety. Only the cytotoxic agent indolinobenzodiazepine (IGN) absorbs at 330 nm, so the concentration at that wavelength can be attributed solely to the cytotoxic agent. The extinction co-efficient values of conjugated moiety are listed in Table 3.

The antibody and cytotoxic agent components were quantified using the following algebraic expressions, which account for the contribution of each constituent at each wavelength:

C_{D}=A₃₃₀/ε_{330 nm IGN}

C_{Ab}=(A₂₈₀−(ε_{280 nm IGN}/ε_{330 nm IGN})×A₃₃₀)/ε_{280 nm Ab}

Aₓ is the absorbance value at X nm wavelength, whereas C_{Ab} is the molar concentration of antibody (i.e., AbX) and C_{D} is the molar concentration of cytotoxic agent. The ratio of cytotoxic agent:Ab (DAR) was calculated as a ratio of the above molar concentrations. The mg/mL (g/L) concentrations of AbX and cytotoxic agent were calculated using the molecular weights listed in Table 3.

TABLE 3

| | MW of | | |
|---|---|---|---|
| | conjugated | ε₂₈₀ | ε₃₃₀ |
| Moiety | moiety* (g/mol) | (M⁻¹cm⁻¹) | (M⁻¹cm⁻¹) |
| AbX antibody | 144,898 | 224,000 | n/a |
| Compound (Ie) | 961 | 30,115 | 15,484 |

### Determining the Percent of Monomeric Conjugate

The percentage of monomeric conjugate in purified AbX-cytotoxic agent samples was determined via HPLC analysis using size-exclusion chromatography (SEC). Approximately 10-100 μg of AbX-cytotoxic agent conjugate was injected onto an HPLC instrument with an attached SEC column (TSK GEL G3000SWx1 5 μm, 7.8 mm×30 cm, Part No. 08541; recommended guard column TSK GEL, 4 cm, Part No. 08543, TOSOH Biosciences, King of Prussia, Pa.), and run at 0.5 mL per minute with an isocratic mobile phase of 400 mM sodium perchlorate, 50 mM sodium phosphate, 5% isopropanol. Absorbance signal was collected for 30 min at 280 nm and 330 nm wavelengths.

AbX antibody monomer typically eluted at ˜17 min, while AbX-cytotoxic agent conjugate monomer often eluted as a doublet with peaks at ˜17 and ˜19 min. High molecular weight species (HMW, e.g., dimer, aggregate) and low molecular weight species (LMW, e.g., fragment) typically eluted at ˜12 and ˜24 min, respectively.

The % monomeric antibody (or conjugate) was calculated from the 280 nm peak area of the 17 min peak (or the 17/19 doublet), and compared to the area of all of the protein peaks combined.

The DAR on the monomer peak was also determined by substituting the peak areas of 280 nm and 330 nm signals into the A₂₈₀ and A₃₃₀ spaces in the C_{D} and C_{Ab} equations shown in the above section, and then dividing C_{D}/C_{Ab}.

### Determining the Percent of Unconjugated Cytotoxic Agent

The amount of unconjugated cytotoxic agent (“free drug”) present in purified conjugate samples was determined via UPLC analysis using tandem SEC and C-18 reverse-phase columns (“dual-column”). Two Waters Acquity UPLC Protein BEH SEC columns (1.7 μm, 4.6×30 mm, Part No. 186005793, Waters Corporation, Milford, Mass.) were connected in series to separate the intact conjugate from free drug, which was then channeled to a Waters Cortecs UPLC C-18 column (2.1×50 mm, Part No. 186007093) to separate and quantify free CDA species. The conjugate was prepared by diluting with acetonitrile (ACN) to 20% (v/v) ACN, injected onto the column series (25 μL), and run according to the gradient listed in Table 4:

TABLE 4

| Time (min) | Flow (mL/min) | % A | % B |
|---|---|---|---|
| 0.0 | 0.35 | 70 | 30 |
| 1.0 | 0.35 | 70 | 30 |
| 8.0 | 0.35 | 20 | 80 |
| 9.0 | 0.35 | 5 | 95 |
| 10.0 | 0.35 | 5 | 95 |
| 10.1 | 0.35 | 70 | 30 |
| 11.0 | 0.35 | 70 | 30 |
| 12.0 | 0.35 | 70 | 30 |
| 13.5 | 0.35 | 70 | 30 |
| 14.0 | 0.35 | 95 | 5 |
| 20.0 | 0.35 | 95 | 5 |
| 21.0 | 0.0 | 95 | 5 |

Table 7: Flow rate = 0.35 ml/min; run time = 12.5 minutes; C-18 column temperature = 30° C.; mobile phases = A: 0.1% (v/v) TFA in water, B: 0.1% (v/v) TFA in ACN

The column was diverted from in-line SEC to C-18 at 2.2 min and back to in-line SEC at 14.0 minutes. Signal was collected at 265 nm. Using a standard curve derived from compound (Ie), the amount of free drug present in the sample was calculated from peaks found in the 2.2-14.0 minute window, using the following formulas:

ng_{free}=(AUC_{265 nm}+11805)/4888%

% free CDA=ng_{free}/ng_{injected}

### Example 4

Conjugates of a humanized antibody Abl and a murine antibody, murine My9-6, with compound (Ie) were prepared according to the protocols described in Example 3. The results are shown in Table 5.

TABLE 5

| | Ab1 | | murine My9-6 | |
|---|---|---|---|---|
| Conjugation scale (mg) | 1.5 | 1.5 | 1.5 | 1.5 |
| Drug excess | 5 | 5 | 5 | 5 |
| Conj. Buffer | 15 mM | 75 mM | 15 mM | 75 mM |
| | HEPES, | EPPS, | HEPES, | EPPS, |
| | pH 8.5 | pH 8 | pH 8.5 | pH 8 |
| DAR (drug to antibody | 2.7 | 2.9 | 4.4 | 4 |
| ratio) | | | | |
| SEC DAR | 2.6 | 2.8 | 2 | 1.8 |
| % monomer | 99.2 | 99.2 | 95.5 | 96.8 |
| Conc. (mg/mL) | 0.5 | 0.7 | 0.4 | 0.5 |
| % Yield | 42 | 57 | 30 | 41 |
| % incorporation | 54 | 58 | 88 | 80 |

As shown in Table 5, conjugation using high ionic strength buffer at pH 8 results in significant increase in reaction yield compared to conjugation using a buffer with low ionic strength at pH 8.5.

The protocol described in Example 3 utilizing 75 mM EPPS, pH 8.0 buffer was used to prepare the 5F9-PVAdG-(Ie) conjugate. The 5F9-PVAdG antibody contains amino acid substitutions that replace ELLG in the heavy chain of IgG1 (SEQ ID NO:9), which are important for binding FcγRIIIb, with PVA, the highly conserved amino acids in IgG2 at the analogous location (Vidarsson et al., IgG subclasses and allotypes: from structure to effector functions, Frontiers in Immunology, 5(520): 1-17(2014)).

The conjugation reaction was carried out using 5F9 PVAdG antibody at 2.0 mg/mL in 75 mM EPPS, pH 8.0 with the addition of sulfonated form of compound (IIe) at a specified molar excess based on the antibody (see Table 6 for representative conjugation). The conjugation reaction had a final 90/10 aqueous:organic composition of 75 mM EPPS, pH 8.0 and DMA, and was incubated in a water bath at 25° C. for 4 hours prior to purification into formulation buffer (10 mM histidine, 50 mM sodium chloride, 8.5% sucrose, 0.01% Tween-20, 50 μM sodium bisulfite, pH 6.2).

The 5F9-PVAdG-(Ie) conjugation reaction mixture was purified using Sephadex G-25 HiPrep columns equilibrated with 10 mM histidine, 50 mM sodium chloride, 8.5% sucrose, 0.01% Tween-20, 50 μM sodium bisulfite, pH 6.2. The purified conjugate was filtered using a 0.22 μm PVDF syringe filter before analysis.

TABLE 6

| | Molar | Conju- | | | |
|---|---|---|---|---|---|
| | Excess of | gation | Conju- | (Ie)/ | |
| | Sulfonated | Scale | gation | Antibody | Monomeric |
| Conjugate | (IIe) | (mg) | Yield | Ratio | Conjugate |
| 5F9- | 3.9 | 98 | 78% | 2.85 | 99% |
| PVAdG- | | | | | |
| (Ie) | | | | | |

### Example 6

### Optimized Sulfonation

Compound (IIe) was sulfonated as follows to generate compound (Ie). To 3.75 mL of a 50 mM sodium succinate, pH 3.3 solution, DMA in the amount of 6.11 mL was added. After mixing and equilibration to 10° C. in a water bath, 1.39 mL of a 21.5 mM compound (IIe) stock solution in DMA (30.0 μmol compound (IIe)) was added and mixed. Following this addition, 3.75 mL of a 20 mM aqueous sodium bisulfite solution (2.5 equivalents, 75 μmol) was introduced into the reaction. After mixing, the reaction was allowed to proceed at 10° C. for 15.5 hours and was used immediately in the next step without purification. Liquid chromatography (reverse phase) analysis of the reaction mixture indicated 92.4% conversion to compound (Ie) with 2.4% remaining unreacted compound (IIe).

### Post Conjugation Quench

In order to determine a condition wherein an increase in ionic strength post conjugation results in a decrease in the formation of the high molecular weight (HMW) species, the following optimization was performed. 5F9 antibody (2 mg/mL) was conjugated to 3.8 molar equivalents of compound (Ie) at 22° C. for 80-90 minutes. The final composition of the conjugation reaction comprised of 130 mM EPPS, pH 8.7 with 15% DMA by volume. Immediately upon completion of the conjugation reaction, aliquots were diluted with the indicated volume of the quench solution as detailed in Table 7. Changes in the percent HMW species were monitored for the indicated time upon holding at 22° C. Based on this finding, 2-fold dilution using 300, 500, or 700 mM EPPS quench solutions, 1.4-1.6-fold dilutions using 750 mM EPPS, and 1.4-1.6-fold dilutions using 750 mM EPPS/150 mM histidine hydrochloride were selected. In the following conjugation example, 1.5-fold dilution using 750 mM EPPS/150 mM histidine hydrochloride was used. Table 7 depicts the effects of quench solutions on the stability of crude 5F9-(Ie) conjugate. Crude 5F9-(Ie) conjugate was incubated with different quench solutions for the specified amount of time and the changes in the percent molecular weight species were determined by size exclusion chromatography.

TABLE 7

| | | | Crude | Quench | Time post | Δ % HMW | |
|---|---|---|---|---|---|---|---|
| | Quench | Fold | reaction | solution | mixing | (initial- | Visible |
| Experiment | Solution | dilution | (mL) | vol. (mL) | (min) | final)* | precipitation |
| 1 | None (control | NA | 0.5 | NA | 720 | 2.0 | no |
| | for experiments | | | | | | |
| | 2-6) | | | | | | |
| 2 | 6.7% w/v % | 2.0 | 0.5 | 0.5 | 760 | NA | yes |
| | sucrose, | | | | | | |
| | 50 uM sodium | | | | | | |
| | bisulfite | | | | | | |
| 3 | 13.4% w/v % | 2.0 | 0.5 | 0.5 | 800 | NA | yes |
| | sucrose, | | | | | | |
| | 50 uM sodium | | | | | | |
| | bisulfite | | | | | | |
| 4 | 20% w/v % | 1.5 | 0.5 | 0.25 | 840 | NA | yes |
| | sucrose, | | | | | | |
| | 100 uM | | | | | | |
| | sodium | | | | | | |
| | bisulfite | | | | | | |
| 5 | 50 mM | 2.0 | 0.5 | 0.5 | 880 | NA | yes |
| | histidine, | | | | | | |
| | 6.7 w/v % | | | | | | |
| | sucrose, 50 | | | | | | |
| | uM sodium | | | | | | |
| | bisulfite, | | | | | | |
| | pH 5.5 | | | | | | |
| 6 | 130 mM | 2.0 | 0.5 | 0.5 | 920 | NA | yes |
| | EPPS, 50 uM | | | | | | |
| | sodium | | | | | | |
| | bisulfite, | | | | | | |
| | pH 8.7 | | | | | | |
| 7 | None (control | NA | 0.5 | NA | 850 | 2.4 | no |
| | for experiments | | | | | | |
| | 8-13) | | | | | | |
| 8 | 400 mM | 1.2 | 0.5 | 0.075 | 890 | 4.2 | no |
| | succinic acid, | | | | | | |
| | 50 uM sodium | | | | | | |
| | bisulfite | | | | | | |
| 9 | 60 mM | 2.0 | 0.5 | 0.5 | 930 | 2.3 | no |
| | succinic acid, | | | | | | |
| | 50 uM sodium | | | | | | |
| | bisulfite | | | | | | |
| 10 | 60 mM | 2.0 | 0.5 | 0.5 | 970 | 4.2 | no |
| | succinic acid, | | | | | | |
| | 13.4% | | | | | | |
| | sucrose, 50 | | | | | | |
| | uM sodium | | | | | | |
| | bisulfite | | | | | | |
| 11 | 300 mM | 2.0 | 0.5 | 0.5 | 1010 | 1.0 | no |
| | EPPS, 50 uM | | | | | | |
| | sodium | | | | | | |
| | bisulfite, | | | | | | |
| | pH 8.7 | | | | | | |
| 12 | 500 mM | 2.0 | 0.5 | 0.5 | 1050 | 0.8 | no |
| | EPPS, 50 uM | | | | | | |
| | sodium | | | | | | |
| | bisulfite, | | | | | | |
| | pH 8.7 | | | | | | |
| 13 | 700 mM | 2.0 | 0.5 | 0.5 | 1090 | 0.6 | no |
| | EPPS, 50 uM | | | | | | |
| | sodium | | | | | | |
| | bisulfite, | | | | | | |
| | pH 8.7 | | | | | | |
| 14 | None (control | NA | 1.0 | NA | 880 | 2.9 | no |
| | experiments | | | | | | |
| | for 15-20) | | | | | | |
| 15 | 600 mM | 1.4 | 1.0 | 0.4 | 600 | 2.8 | no |
| | histidine | | | | | | |
| | hydrochloride | | | | | | |
| 16 | 600 mM | 1.5 | 1.0 | 0.5 | 640 | 3.2 | no |
| | histidine | | | | | | |
| | hydrochloride | | | | | | |
| 17 | 600 mM | 1.6 | 1.0 | 0.6 | 680 | 3.6 | no |
| | histidine | | | | | | |
| | hydrochloride | | | | | | |
| 18 | 600 mM | 1.4 | 1.0 | 0.4 | 720 | 2.7 | no |
| | histidine | | | | | | |
| | hydrochloride, | | | | | | |
| | 20 w/v % | | | | | | |
| | sucrose | | | | | | |
| 19 | 600 mM | 1.5 | 1.0 | 0.5 | 760 | 2.9 | no |
| | histidine | | | | | | |
| | hydrochloride, | | | | | | |
| | 20 w/v % | | | | | | |
| | sucrose | | | | | | |
| 20 | 600 mM | 1.6 | 1.0 | 0.6 | 800 | 2.9 | no |
| | histidine | | | | | | |
| | hydrochloride, | | | | | | |
| | 20 w/v % | | | | | | |
| | sucrose | | | | | | |
| 21 | None (control | NA | 1.0 | NA | 840 | 2.2 | no |
| | experiments | | | | | | |
| | for 22-27) | | | | | | |
| 22 | 750 mM | 1.4 | 1.0 | 0.4 | 600 | 0.4 | no |
| | EPPS | | | | | | |
| 23 | 750 mM | 1.5 | 1.0 | 0.5 | 640 | 0.5 | no |
| | EPPS | | | | | | |
| 24 | 750 mM | 1.6 | 1.0 | 0.6 | 680 | 0.4 | no |
| | EPPS | | | | | | |
| 25 | 750 mM | 1.4 | 1.0 | 0.4 | 720 | 0.5 | no |
| | EPPS, 150 | | | | | | |
| | mM histidine | | | | | | |
| | hydrochloride | | | | | | |
| 26 | 751 mM | 1.5 | 1.0 | 0.5 | 760 | 0.5 | no |
| | EPPS, 150 | | | | | | |
| | mM histidine | | | | | | |
| | hydrochloride | | | | | | |
| 27 | 752 mM | 1.6 | 1.0 | 0.6 | 800 | 0.5 | no |
| | EPPS, 150 | | | | | | |
| | mM histidine | | | | | | |
| | hydrochloride | | | | | | |

*Calculated by subtracting the % HMW of the appropriate control at t = 0 min from the experiment % HMW at the time indicated in the table.

### Optimized Conjugation and Purification

In a 1 L jacketed glass reactor equipped with an overhead stirrer containing 325 mL of 130 mM EPPS, pH 8.7, 68.6 mL of DMA was added. Following mixing and equilibration of the solution to 22° C., 100 mL of a 10.0 mg/mL solution of 5F9 antibody in 130 mM EPPS, pH 8.7 was introduced into the reactor and allowed to mix for 15 minutes. Subsequently 12.8 mL of the 2 mM compound (Ie) solution (25.5 μmol, 3.7 equivalents of 5F9 antibody; prepared using the optimized sulfonation protocol described previously) was introduced into the reaction solution. After stirring for 60 min at 22° C., 250 mL of an aqueous solution containing 150 mM histidine hydrochloride and 750 mM EPPS was transferred into the reaction vessel. Subsequent to mixing thoroughly, this material was filtered through a Millipore Optiscale 47 Express SHC 0.5/0.2 μM filter. The crude reaction mixture was then concentrated by ultrafiltration with a TangenX 0.02 m² HyStream 30 kD Sius LSN TFF cassette to a calculated bulk protein concentration of 2.5 mg/mL. Following the concentration step, the solution was diafiltered against 4.8 L of a 50 mM histidine, 6.7 w/v (weight/volume) % sucrose, 0.1 v/v (volume/volume) % polysorbate-80, 50 μM sodium bisulfite, pH 5.5 buffer. After diafiltration, polysorbate-80 was added to the retentate solution at a final concentration of 0.1 v/v (volume/volume) % polysorbate-80 and the resulting solution was filtered with a Millipore Optiscale 47 Express SHC 0.5/0.2 μM filter. Following storage at 2-8° C. for 2 d, the solution was diluted to 1.0 mg/mL conjugate by addition of the necessary volume of additional 50 mM histidine, 6.7 w/v % sucrose, 0.1 v/v % polysorbate-80, 50 μM sodium bisulfite, pH 5.5 buffer. This solution was then filtered through a Millipore Optiscale 47 Durapore 0.22 μM filter giving 818 mL of 1.0 mg/mL conjugate. The measured DAR of the final conjugate is 2.6 by UV/vis with 97.4% monomer and 2.5% HMW by SEC. The final yield of the product was 82%.

### Analytical:

The concentration of antibody and cytotoxic agent (Ie) in purified conjugate samples was determined by UV/Vis using absorbance values at 280 nm and 330 nm. Since both the antibody and the cytotoxic agent absorb at 280 nm, a binomial equation was required to consider the portion of total signal attributed to each moiety. Only the cytotoxic agent indolinobenzodiazepine (IGN) absorbs at 330 nm, so the concentration at that wavelength can be attributed solely to the cytotoxic agent. The extinction co-efficient values of conjugated moiety used in this example are 34150 and 16270 M⁻¹cm⁻¹ at 280 and 330 nm, respectively.

The antibody and cytotoxic agent components were quantified using the following algebraic expressions, which account for the contribution of each constituent at each wavelength:

C_{D}=A₃₃₀/ε_{330 nm IGN}

C_{Ab}=(A₂₈₀−(ε_{280 nm IGN}/ε_{330 nm IGN})×A₃₃₀)/ε_{280 nm Ab}

Aₓ is the absorbance value at X nm wavelength, whereas C_{Ab} is the molar concentration of antibody (i.e., AbX) and C_{D} is the molar concentration of cytotoxic agent. The ratio of cytotoxic agent:Ab (DAR) was calculated as a ratio of the above molar concentrations. The mg/mL (g/L) concentration of AbX was calculated using a molecular weight of 144887 g/mol.

We claim:

1. A method of preparing a cell-binding agent-cytotoxic agent conjugate comprising the step of reacting a cell-binding agent with a cytotoxic agent or a cytotoxic agent-linker compound having a reactive group capable of forming a covalent bond with the cell-binding agent at a pH between 4 to 9 in the presence of a buffer solution with high ionic strength, wherein the cell-binding agent comprises a lysine ε—NH₂ group that forms a covalent bond with the cytotoxic agent or the cytotoxic agent-linker compound having an amine-reactive group.

2. The method of claim 1, wherein the pH is between 7.3 and 8.7.

3. The method of claim 1, wherein the pH is between 7.3 and 8.4.

4. The method of claim 1, wherein the pH is between 7.6 and 8.4.

5. The method of claim 1, wherein the pH is between 7.7 and 8.3

6. The method of claim 1, wherein the pH is between 7.8 and 8.2.

7. The method of claim 1, wherein the pH is between 7.9 and 8.1.

8. The method of claim 1, wherein the pH is at 8.0.

9. The method of claim 1, wherein the pH is between 8.5 to 8.9.

10. The method of claim 1, wherein the pH is between 8.6 to 8.8.

11. The method of claim 1, wherein the pH is 8.7.

12. The method of any one of claims 1-11, wherein the buffer solution has an ionic strength of 20 mM to 500 mM.

13. The method of claim 12, wherein the buffer solution has an ionic strength of 50 mM to 100 mM.

14. The method of claim 12, wherein the buffer solution has an ionic strength of 60 mM to 90 mM.

15. The method of claim 12, wherein the buffer solution has an ionic strength of 70 mM to 80 mM.

16. The method of claim 12, wherein the buffer solution has an ionic strength of 75 mM.

17. The method of claim 12, wherein the buffer solution has an ionic strength of 100 nM to 200 mM.

18. The method of claim 12, wherein the buffer solution has an ionic strength of 100 nM to 160 nM.

19. The method of claim 12, wherein the buffer solution has an ionic strength of 120 nM to 140 nM.

20. The method of claim 12, wherein the buffer solution has an ionic strength of 130 nM.

21. The method of claim 1, wherein the buffer solution has a pH between 7.8 to 8.9 and an ionic strength between 50 mM and 200 mM.

22. The method of claim 1, wherein the buffer has a pH between 7.8 to 8.2 and an ionic strength between 70 mM and 80 mM.

23. The method of claim 1, wherein the buffer has a pH of 8.0 and an ionic strength of 75 mM.

24. The method of claim 1, wherein the buffer has a pH between 8.5 to 8.9 and an ionic strength between 120 mM to 140 mM.

25. The method of claim 1, wherein the buffer has a pH of 8.7 and an ionic strength of 130 mM.

26. The method of any one of claims 1-25, wherein the buffer solution is selected from the group consisting of MES ((2-(N-morpholino)ethanesulfonic acid)) buffer, bis-tris methane (2-[Bis(2-hydroxyethyl)amino]-2-(hydroxymethyl)propane-1,3-diol) buffer, ADA (N-(2-Acetamido)iminodiacetic acid) buffer, ACES (N-2-aminoethanesulfonic acid) buffer, PIPES (piperazine-N,N′-bis(2-ethanesulfonic acid)), MOPSO (β-Hydroxy-4-morpholinepropanesulfonic acid) buffer, bis-tris propane (1,3-bis(tris(hydroxymethyl)methylamino)propane) buffer, BES (N,N-bis(2-hydroxyethyl)-2-aminoethanesulfonic acid), TES (N-[tris(hydroxymethyl)methyl]-2-aminoethanesulfonic acid) buffer, HEPES (4-(2-hydroxyethyl)piperazine-1-ethanesulfonic acid) buffer, DIPSO β-(N,N-Bis[2-hydroxyethyl]amino)-2-hydroxypropanesulfonic acid or N,N-Bis(2-hydroxyethyl)-3-amino-2-hydroxypropanesulfonic acid), MOBS (4-(N-morpholino)butanesulfonic acid) buffer, TAPSO β-[[1,3-dihydroxy-2-(hydroxymethyl)propan-2-yl]amino]-2-hydroxypropane-1-sulfonic acid) buffer, trizma (Tris or 2-Amino-2-(hydroxymethyl)-1,3-propanediol) buffer, HEPPSO (N-(2-hydroxyethyl)piperazine-N′-(2-hydroxypropanesulfonic acid)) buffer, POPSO (piperazine-1,4-bis-(2-hydroxy-propane-sulfonic acid) dehydrate) buffer, EPPS (4-(2-hydroxyethyl)piperazine-1-propanesulfonic acid) buffer, tricine (N-(2-Hydroxy-1,1-bis(hydroxymethyl)ethyl)glycine) bufer, gly-gly, bicine (2-(Bis(2-hydroxyethyl)amino)acetic acid) buffer, HEPBS (N-(2-Hydroxyethyl)piperazine-N′-(4-butanesulfonic acid)) buffer, TAPS β-[[1,3-dihydroxy-2-(hydroxymethyl)propan-2-yl]amino]propane-1-sulfonic acid) buffer, AMPD (2-amino-2-methyl-1,3-propanediol) buffer, TABS (N-tris(Hydroxymethyl)methyl-4-aminobutanesulfonic acid) buffer, AMPSO (N-(1,1-Dimethyl-2-hydroxyethyl)-3-amino-2-hydroxypropanesulfonic acid) buffer and a combination thereof.

27. The method of claim 26, wherein the buffer solution is an EPPS buffer.

28. The method of claim 1, wherein the buffer solution is 50 mM to 200 mM EPPS buffer having a pH between 7.8 and 8.9.

29. The method of claim 1, wherein the buffer solution is 70 mM to 80 mM EPPS buffer having a pH between 7.8 and 8.2.

30. The method of claim 1, wherein the buffer solution is 75 mM EPPS buffer having a pH of 8.0.

31. The method of claim 1, wherein the buffer solution is 120 mM to 140 mM EPPS buffer having a pH between 8.5 and 8.9.

32. The method of claim 1, wherein the buffer solution is 130 mM EPPS buffer having a pH of 8.7.

33. A method of preparing a cell-binding agent-cytotoxic agent conjugate comprising the step of reacting a cell-binding agent with a cytotoxic agent or a cytotoxic agent-linker compound in a buffer solution having a pH of 7.3 to 9.0, wherein the cell-binding agent comprises a lysine ε—NH₂ group that forms a covalent bond with the cytotoxic agent or the cytotoxic agent-linker compound having an amine-reactive group.

34. The method of claim 33, wherein the pH of the buffer solution is between 7.3 and 8.4.

35. The method of claim 33, wherein the pH is between 7.6 and 8.4.

36. The method of claim 33, wherein the pH is between 7.7 and 8.3

37. The method of claim 33, wherein the pH is between 7.8 and 8.2.

38. The method of claim 33, wherein the pH is between 7.9 and 8.1.

39. The method of claim 33, wherein the pH is at 8.0.

40. The method of claim 33, wherein the pH is between 8.5 and 8.9.

41. The method of claim 33, wherein the pH is between 8.6 and 8.8.

42. The method of claim 33, wherein the pH is 8.7.

43. A method of preparing a cell-binding agent-cytotoxic agent conjugate comprising the step of reacting a cell-binding agent with a cytotoxic agent or a cytotoxic agent-linker compound having a reactive group capable of forming a covalent bond with the cell-binding agent at a pH between 4 to 9 in the presence of a high concentration buffer solution, wherein the cell-binding agent comprises a lysine ε—NH₂ group that forms a covalent bond with the cytotoxic agent or the cytotoxic agent-linker compound having an amine-reactive group.

44. The method of claim 43, wherein the concentration of the buffer solution is between 20 mM and 750 mM, between 20 mM and 500 mM, 20 mM and 200 mM, between 25 mM and 150 mM, between 50 mM and 150 mM, between 50 mM and 100 mM, between 100 mM and 200 mM, or between 100 mM and 150 mM.

45. The method of claim 43 or 44, wherein the pH is between 7.3 and 8.9, 7.3 and 8.4, between 7.6 and 8.4, between 7.7 and 8.3, between 7.8 and 8.2, 8.5 and 8.9, or between 8.6 and 8.8.

46. The method of claim 43, wherein the buffer solution has a concentration between 20 mM and 200 mM and a pH between 7.1 and 8.5.

47. The method of claim 43, wherein the buffer solution has a concentration between 50 mM and 150 mM and a pH between 7.6 and 8.4.

48. The method of claim 43, wherein the buffer solution has a concentration between 50 mM and 100 mM and a pH between 7.7 and 8.3.

49. The method of claim 43, wherein the buffer solution has a concentration between 60 mM and 90 mM and a pH between 7.8 and 8.2.

50. The method of claim 43, wherein, the buffer solution has a concentration between 70 mM and 80 mM and a pH between 7.9 and 8.1.

51. The method of claim 43, wherein the buffer solution has a concentration between 50 mM and 200 mM and a pH between 7.8 and 8.9.

52. The method of claim 43, wherein the buffer solution has a concentration between 110 mM and 150 mM and a pH between 8.5 and 8.9.

53. The method of claim 43, wherein the buffer solution has a concentration between 120 mM and 140 mM and a pH between 8.6 and 8.8.

54. The method of any one of claims 33-53, wherein the buffer solution is selected from the group consisting of MES ((2-(N-morpholino)ethanesulfonic acid)) buffer, bis-tris methane (2-[Bis(2-hydroxyethyl)amino]-2-(hydroxymethyl)propane-1,3-diol) buffer, ADA (N-(2-Acetamido)iminodiacetic acid) buffer, ACES (N-2-aminoethanesulfonic acid) buffer, PIPES (piperazine-N,N′-bis(2-ethanesulfonic acid)), MOPSO (β-Hydroxy-4-morpholinepropanesulfonic acid) buffer, bis-tris propane (1,3-bis(tris(hydroxymethyl)methylamino)propane) buffer, BES (N,N-bis(2-hydroxyethyl)-2-aminoethanesulfonic acid), TES (N-[tris(hydroxymethyl)methyl]-2-aminoethanesulfonic acid) buffer, HEPES (4-(2-hydroxyethyl)piperazine-1-ethanesulfonic acid) buffer, DIPSO β-(N,N-Bis[2-hydroxyethyl]amino)-2-hydroxypropanesulfonic acid or N,N-Bis(2-hydroxyethyl)-3-amino-2-hydroxypropanesulfonic acid), MOBS (4-(N-morpholino)butanesulfonic acid) buffer, TAPSO β-[[1,3-dihydroxy-2-(hydroxymethyl)propan-2-yl]amino]-2-hydroxypropane-1-sulfonic acid) buffer, trizma (Tris or 2-Amino-2-(hydroxymethyl)-1,3-propanediol) buffer, HEPPSO (N-(2-hydroxyethyl)piperazine-N′-(2-hydroxypropanesulfonic acid)) buffer, POPSO (piperazine-1,4-bis-(2-hydroxy-propane-sulfonic acid) dehydrate) buffer, EPPS (4-(2-hydroxyethyl)piperazine-1-propanesulfonic acid) buffer, tricine (N-(2-Hydroxy-1,1-bis(hydroxymethyl)ethyl)glycine) bufer, gly-gly, bicine (2-(Bis(2-hydroxyethyl)amino)acetic acid) buffer, HEPBS (N-(2-Hydroxyethyl)piperazine-N′-(4-butanesulfonic acid)) buffer, TAPS β-[[1,3-dihydroxy-2-(hydroxymethyl)propan-2-yl]amino]propane-1-sulfonic acid) buffer, AMPD (2-amino-2-methyl-1,3-propanediol) buffer, TABS (N-tris(Hydroxymethyl)methyl-4-aminobutanesulfonic acid) buffer, AMPSO (N-(1,1-Dimethyl-2-hydroxyethyl)-3-amino-2-hydroxypropanesulfonic acid) buffer and a combination thereof.

55. The method of claim 54, wherein the buffer solution is an EPPS buffer.

56. The method of claim 54, wherein the buffer solution is a 75 mM EPPS buffer.

57. The method of claim 54, wherein the buffer solution is a 130 mM EPPS buffer.

58. The method of any one of claims 1-57, further comprises the step of mixing a quenching solution with high ionic strength after the reaction of the cytotoxic agent or the cytotoxic agent-linker compound with the cell-binding agent.

59. The method of claim 58, wherein the quenching solution has an ionic strength between 200 mM and 3000 mM, between 200 mM and 2000 mM, between 200 mM and 1000 mM, between 500 mM and 1000 mM, between 550 mM and 1000 mM, or between 600 mM and 1000 mM.

60. The method of claim 58, wherein the quenching solution has an ionic strength between 700 mM and 1000 mM.

61. The method of any one of claims 58-60, wherein the quenching solution comprises EPPS.

62. The method of any one of claims 58-60, wherein the quenching solution comprises EPPS and histidine hydrochloride.

63. The method of any one of claims 1-57, further comprises the step of mixing a quenching solution comprising a high centration buffer after the reaction of the cytotoxic agent or the cytotoxic agent-linker compound with the cell-binding agent.

64. The method of claim 63, wherein the concentration of the buffer in the quenching solution is between 200 mM and 3000 mM, between 200 nM and 2000 mM, between 200 mM and 1000 mM, between 500 mM and 1000 mM, between 550 mM and 1000 mM, or between 600 mM and 1000 mM.

65. The method of claim 63 or 64, wherein subsequent to the mixing, the final concentration of the buffer is between 150 mM and 750 mM, between 150 mM and 600 mM, between 200 mM and 500 nM, between 200 mM and 400 nM, or between 250 mM and 350 mM.

66. The method of any one of claims 58-65, wherein the quenching solution has a pH between 5 to 9.

67. The method of claim 66, wherein the quenching solution has a pH between 5 to 7.

68. The method of claim 66, wherein the quenching solution has pH between 5 to 6.

69. The method of claim 66, wherein the quenching solution has a pH of 5.5.

70. The method of claim 69, wherein the quenching solution comprises 750 mM EPPS and 150 mM of histidine hydrochloride.

71. The method of any one of claims 58-70, wherein the addition of the quenching buffer reduces the amount of high molecular weight species.

72. The method of any one of claims 1-71, wherein the cytotoxic agent or the cytotoxic agent-linker compound is represented by one of the following structural formulas: 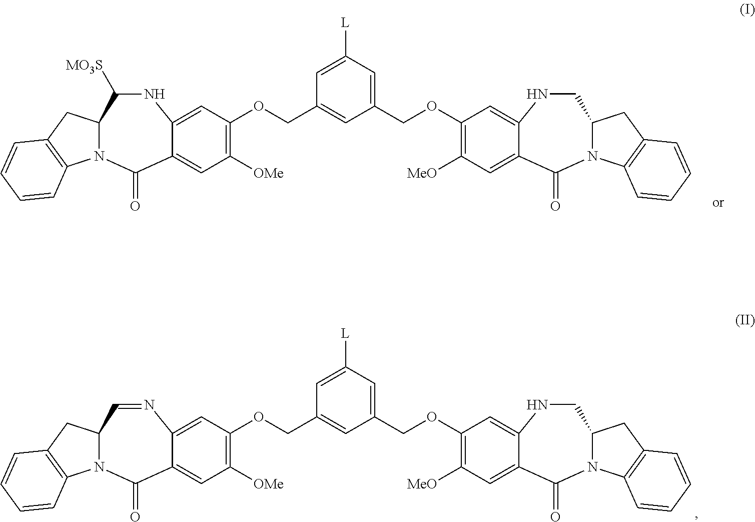
or a pharmaceutically acceptable salt thereof, wherein:
L is represented by the following formula:
—NR₅-P—C(═O)—(CRₐR_{b})ₘ—C(═O)E (A1); or
—NR₅-P—C(═O)—(CRₐR_{b})ₘ—S—Z^{s1} (A3);
wherein:
R₅ is —H or a (C₁-C₃)alkyl;
P is an amino acid residue or a peptide containing between 2 to 20 amino acid residues;
Rₐ and R_{b}, for each occurrence, are each independently —H, (C₁-C₃)alkyl, or a charged substituent or an ionizable group Q;
m is an integer from 1 to 6; and
Z^{s1} is selected from any one of the following formulas: 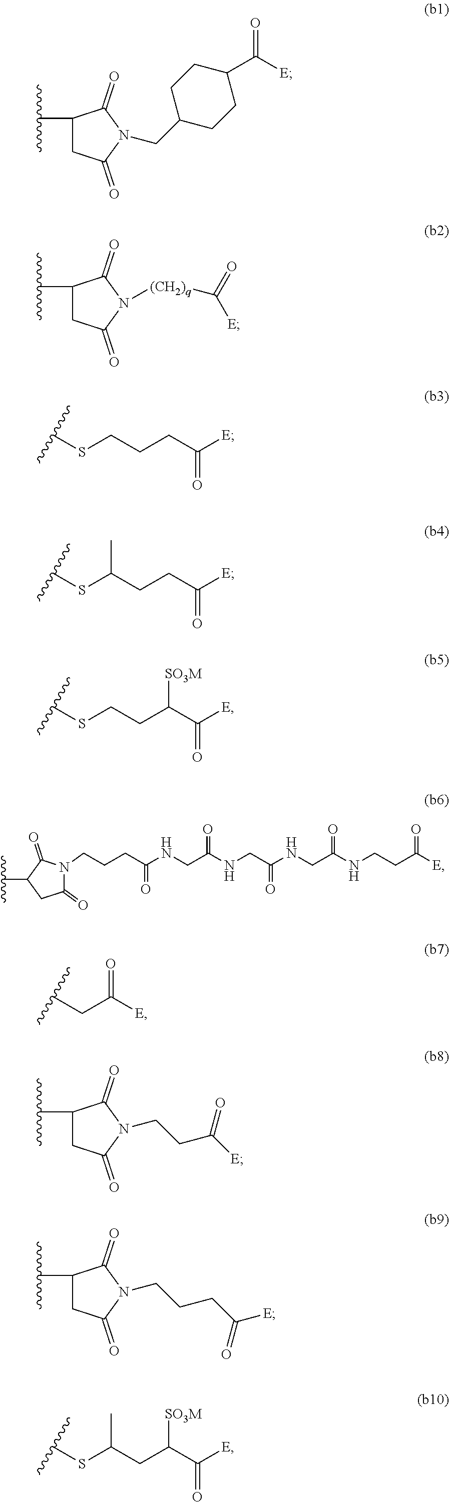
wherein:
q is an integer from 1 to 5;
M is —H or a cation; and
—C(═O)E represents a reactive ester group.

73. The method of claim 72, wherein Rₐ and R_{b} are both H; and R₅ is H or Me.

74. The method of claim 72 or 73, wherein P is a peptide containing 2 to 5 amino acid residues.

75. The method of claim 74, wherein P is a peptide cleavable by a protease.

76. The method of claim 75, wherein P is a peptide cleavable by a protease expressed in tumor tissue.

77. The method of any one of claims 72-74, wherein P is selected from Gly-Gly-Gly, Ala-Val, Val-Ala, Val-Cit, Val-Lys, Phe-Lys, Lys-Lys, Ala-Lys, Phe-Cit, Leu-Cit, Ile-Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu (SEQ ID NO:21), β-Ala-Leu-Ala-Leu (SEQ ID NO:22), Gly-Phe-Leu-Gly (SEQ ID NO:23), Val-Arg, Arg-Val, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, and Met-Ala.

78. The method of claim 77, wherein P is Gly-Gly-Gly, Ala-Val, Ala-Ala, Ala-D-Ala, D-Ala-Ala, or D-Ala-D-Ala.

79. The method of any one of claims 72-78, wherein Q is —SO₃M.

80. The method of claim 72, wherein the cytotoxic agent or the cytotoxic agent-linker compound is represented by one of the following formulas: 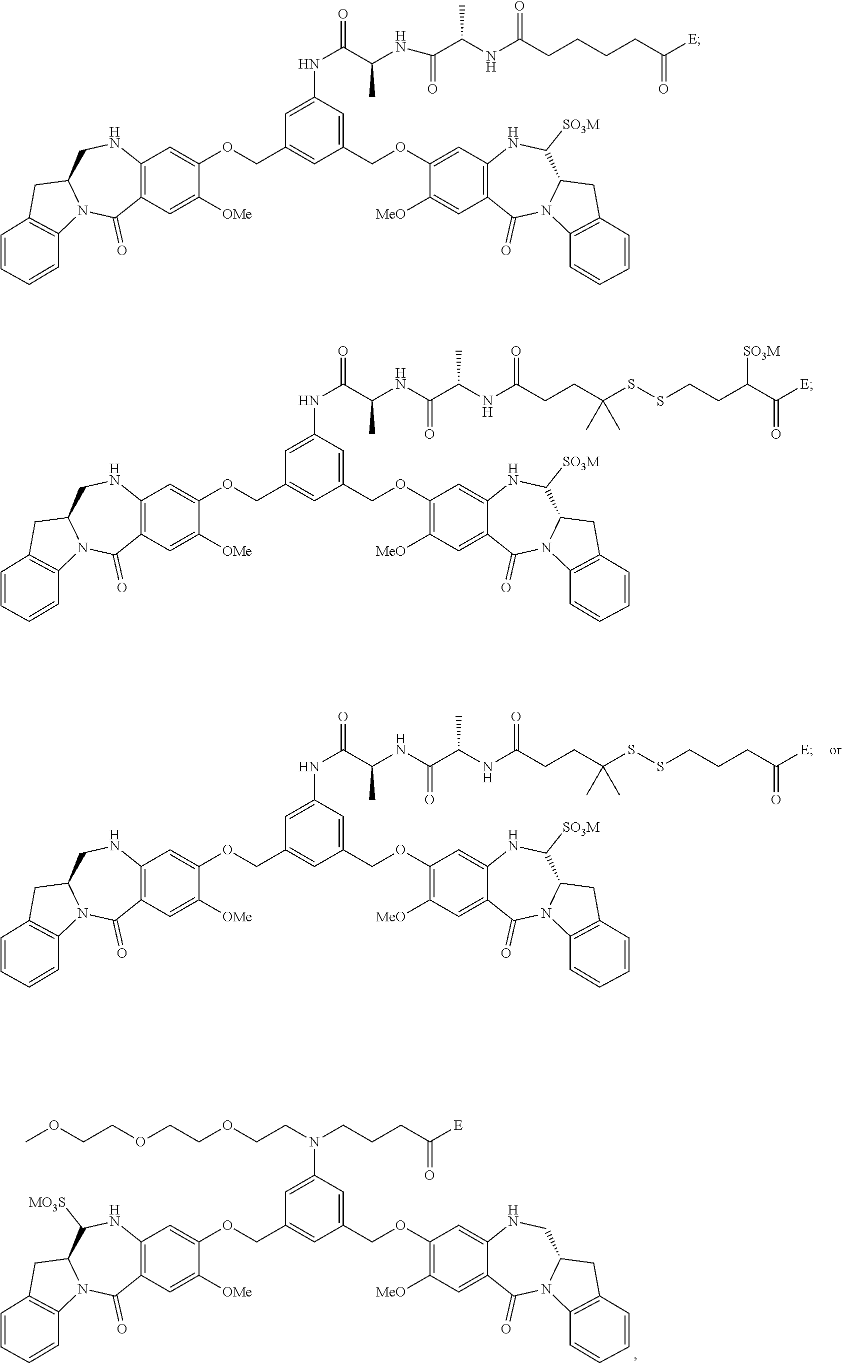
or a pharmaceutically acceptable salt thereof.

81. The method of claim 72, wherein the cytotoxic agent or the cytotoxic agent-linker compound is represented by one of the following structural formulas: 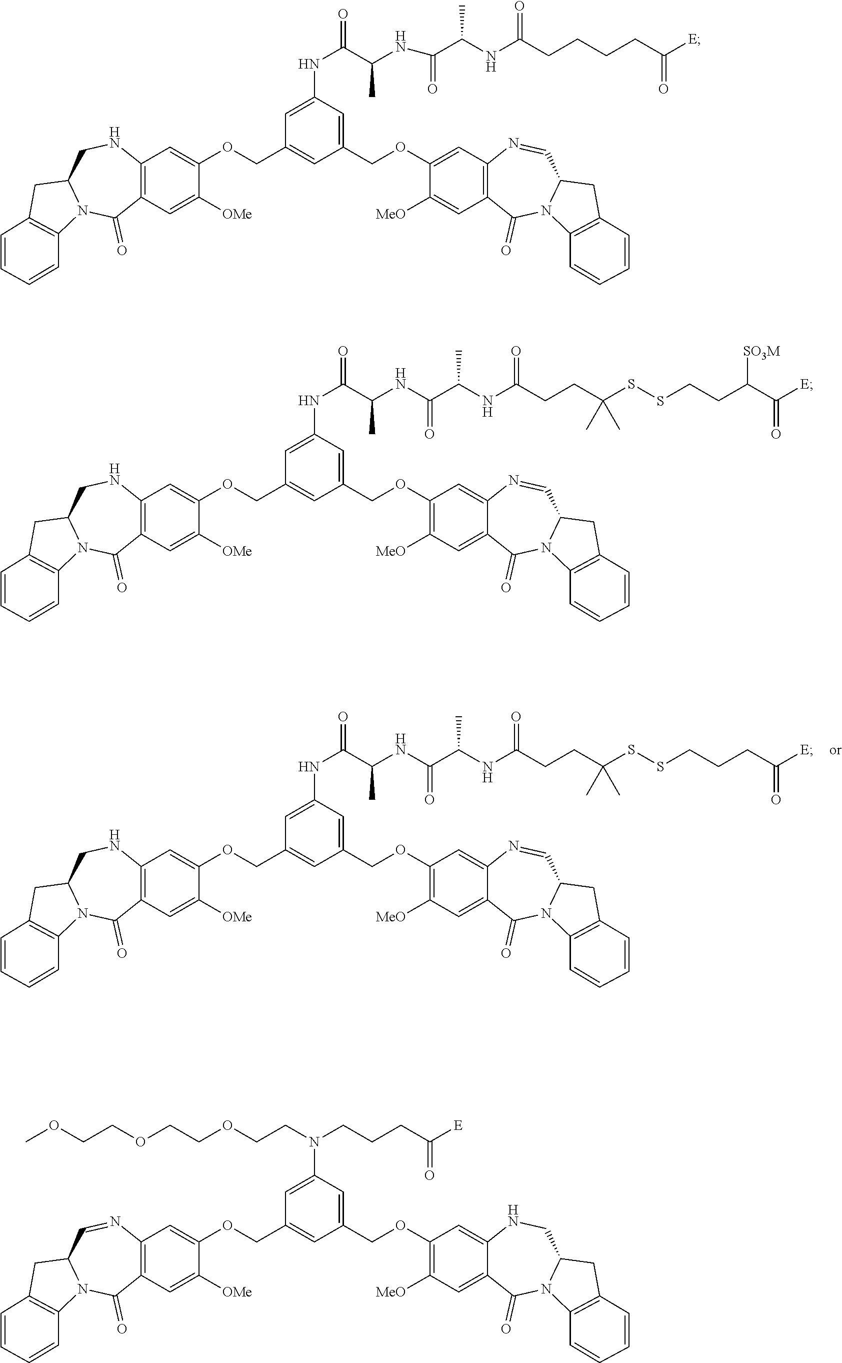
or a pharmaceutically acceptable salt thereof.

82. The method of any one of claims 72-81, wherein the reactive ester group is selected from N-hydroxysuccinimide ester, N-hydroxy sulfosuccinimide ester, nitrophenyl (e.g., 2 or 4-nitrophenyl) ester, dinitrophenyl (e.g., 2,4-dinitrophenyl) ester, sulfo-tetrafluorophenyl (e.g., 4-sulfo-2,3,5,6-tetrafluorophenyl) ester, and pentafluorophenyl ester.

83. The method of claim 82, wherein the reactive ester group is represented by the following formula: 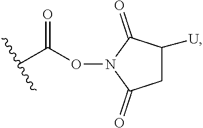
wherein U is H or —SO₃M.

84. The method of claim 82, wherein the reactive ester group is represented by the following formula:

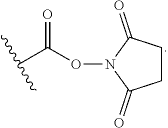 [SMILES: *C(=O)ON1C(=O)CCC1=O]

85. The method of claim 72, wherein the cytotoxic agent is represented by the following structural formula: 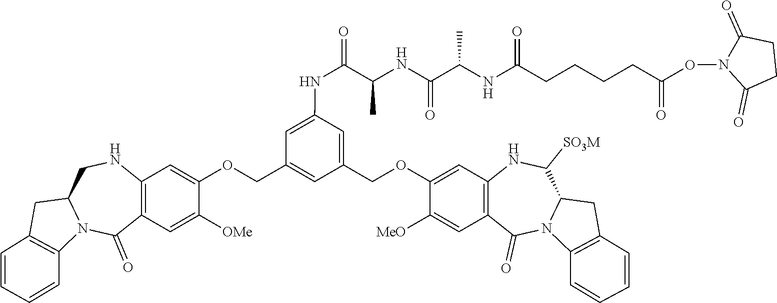
or a pharmaceutically acceptable salt thereof.

86. The method of claim 72, wherein the cytotoxic agent is represented by the following structural formula: 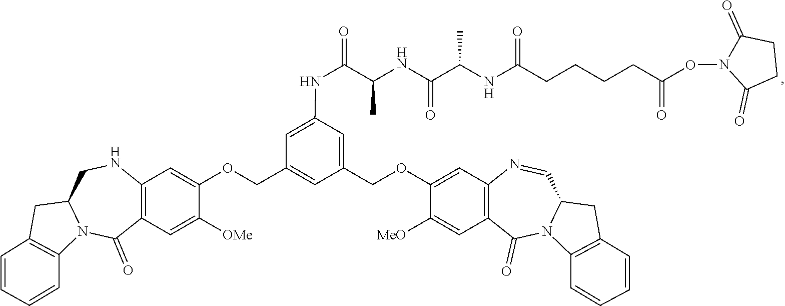 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)ON3C(=O)CCC3=O)c1)N=C[C@@H]1Cc3ccccc3N1C2=O]
or a pharmaceutically acceptable salt thereof.

87. The method of any of claims 72-79 and 82-84, wherein the cytotoxic agent or the cytotoxic agent-linker compound is represented by structural formula (I) prepared by reacting the compound of structural formula (II) with a sulfonating reagent.

88. The method of any one of claims 80 and 82-84, wherein the cytotoxic agent or the cytotoxic agent-linker compound is prepared by reacting a compound represented by one of the following structural formulas: 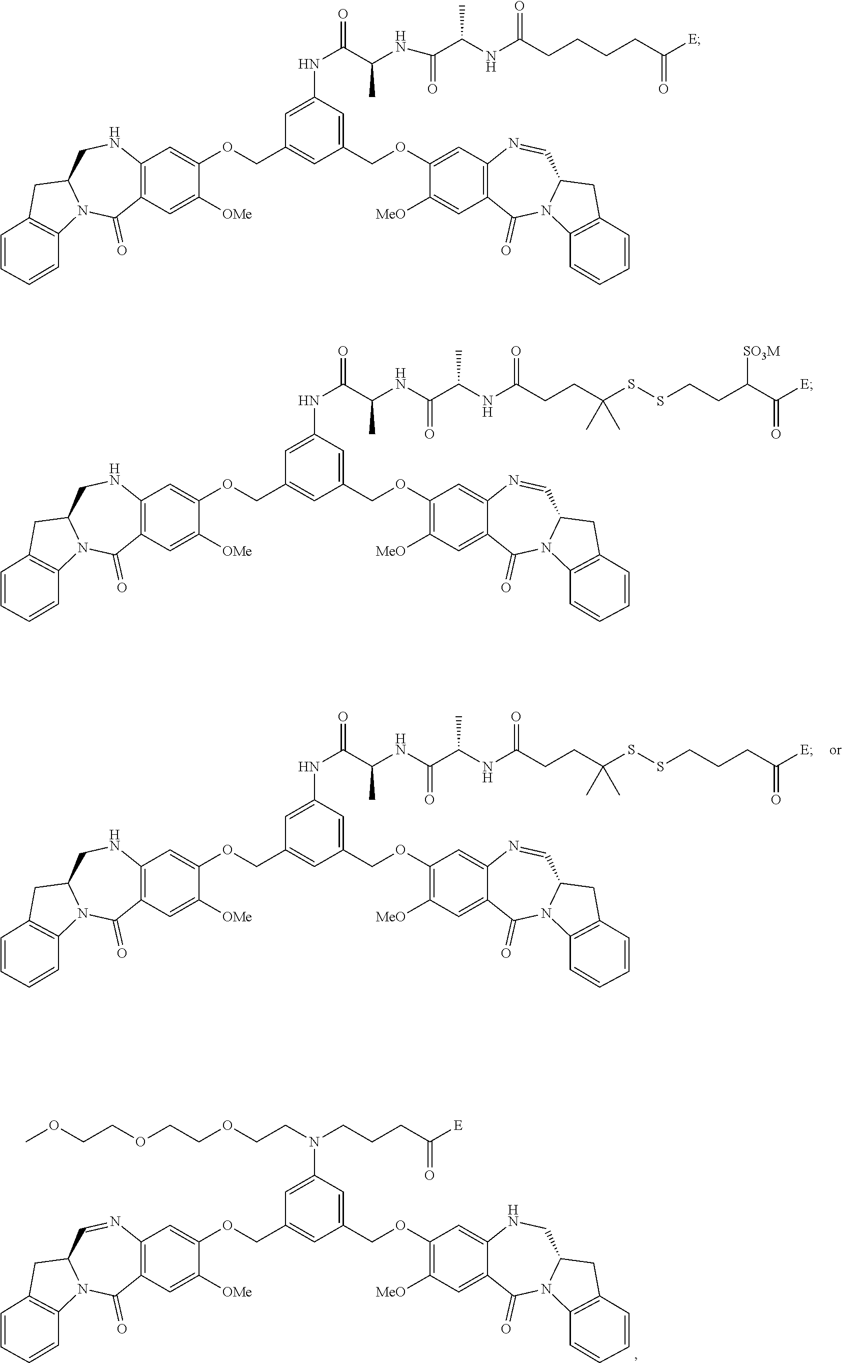
or a pharmaceutically acceptable salt thereof, with a sulfonating reagent.

89. The method of claim 85, wherein the cytotoxic agent is prepared reacting a compound represented by one of the following formulas: 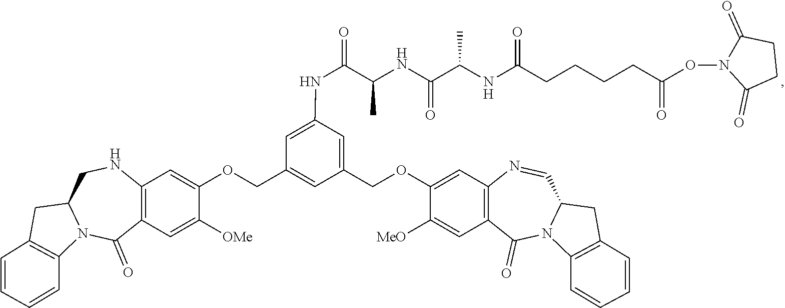 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)ON3C(=O)CCC3=O)c1)N=C[C@@H]1Cc3ccccc3N1C2=O]
or a pharmaceutically acceptable salt thereof, with a sulfonating reagent.

90. The method of any of one of claims 72-79 and 82-84, wherein the cytotoxic agent or the cytotoxic agent-linker compound is represented by structural formula (II), and wherein the method further comprises reacting the cell-binding agent-cytotoxic agent conjugate with a sulfonating reagent.

91. The method of any of one of claims 72-79 and 82-84, wherein the cytotoxic agent or the cytotoxic agent-linker compound is represented by structural formula (II), and wherein the method comprises reacting the cell-binding agent with the cytotoxic agent or the cytotoxic agent-linker compound represented by structural formula (II) in the presence of a sulfonating reagent.

92. The method of any one of claims 81-84 and 86, wherein the method further comprises reacting the cell-binding agent-cytotoxic agent conjugate with a sulfonating reagent.

93. The method of any one of claims 81-84 and 86, wherein the method comprises reacting the cell-binding agent with the cytotoxic agent or the cytotoxic agent-linker compound in the presence of a sulfonating reagent.

94. The method of any one of claims 1-71, wherein the cytotoxic agent or the cytotoxic agent-linker compound is represented by one of the following structural formulas: 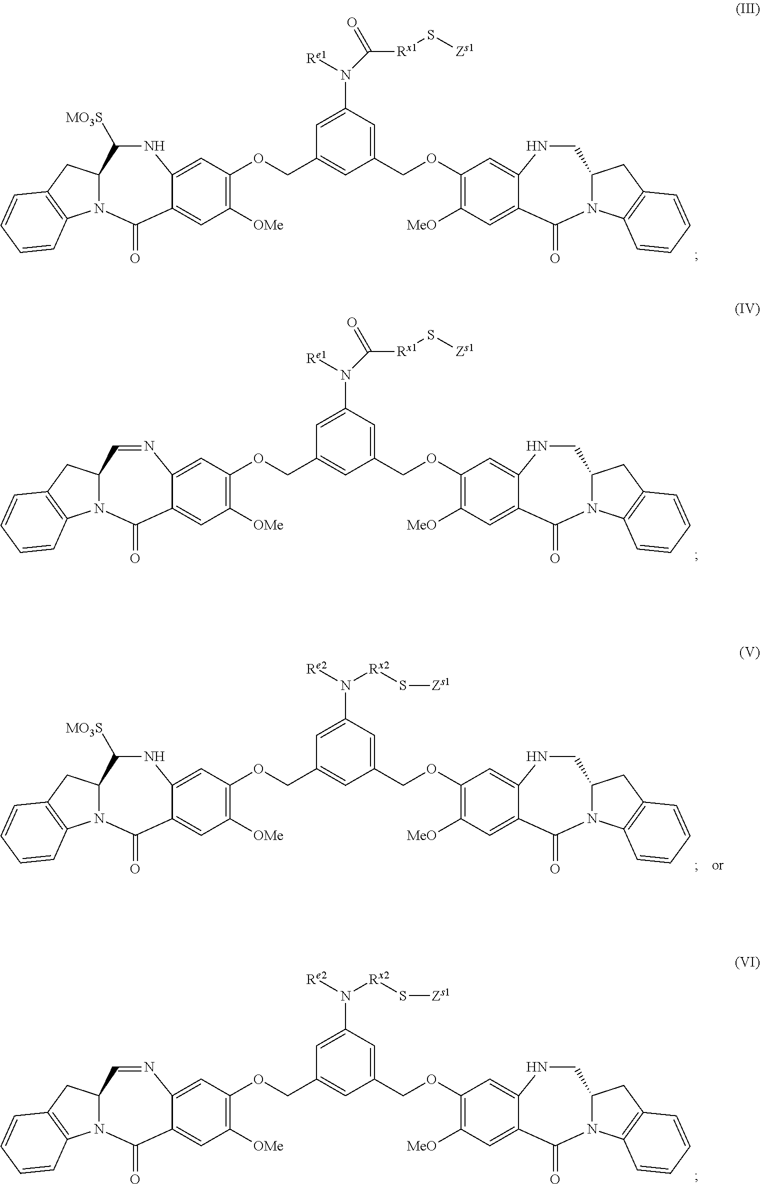
or a pharmaceutically acceptable salt thereof, wherein:
R^{x1} and R^{x2} are independently (C₁-C₆)alkyl;
R^{e1} is —H or a (C₁-C₆)alkyl;
R^{e2} is —(CH₂—CH₂—C₆)ₙ—R^{k};
n is an integer from 2 to 6;
R^{k} is —H or -Me;
Z^{s1} is selected from any one of the following formulas: 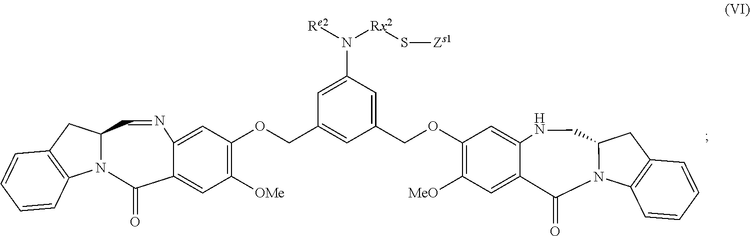
wherein:
q is an integer from 1 to 5;
M is —H or a cation; and
C(═O)E represents a reactive ester group.

95. The method of claim 94, wherein R¹ is H or Me; R^{x1} and R^{x2} are independently —(CH₂)ₚ—(CR^{f}R^{g})—, wherein R^{f} and R^{g} are each independently —H or a (C₁-C₄)alkyl; and p is 0, 1, 2 or 3.

96. The method of claim 95, wherein R^{f} and R^{g} are the same or different, and are selected from —H and -Me.

97. The method of claim 94, wherein the cytotoxic agent or the cytotoxic agent-linker compound is represented by one of the following formulas: 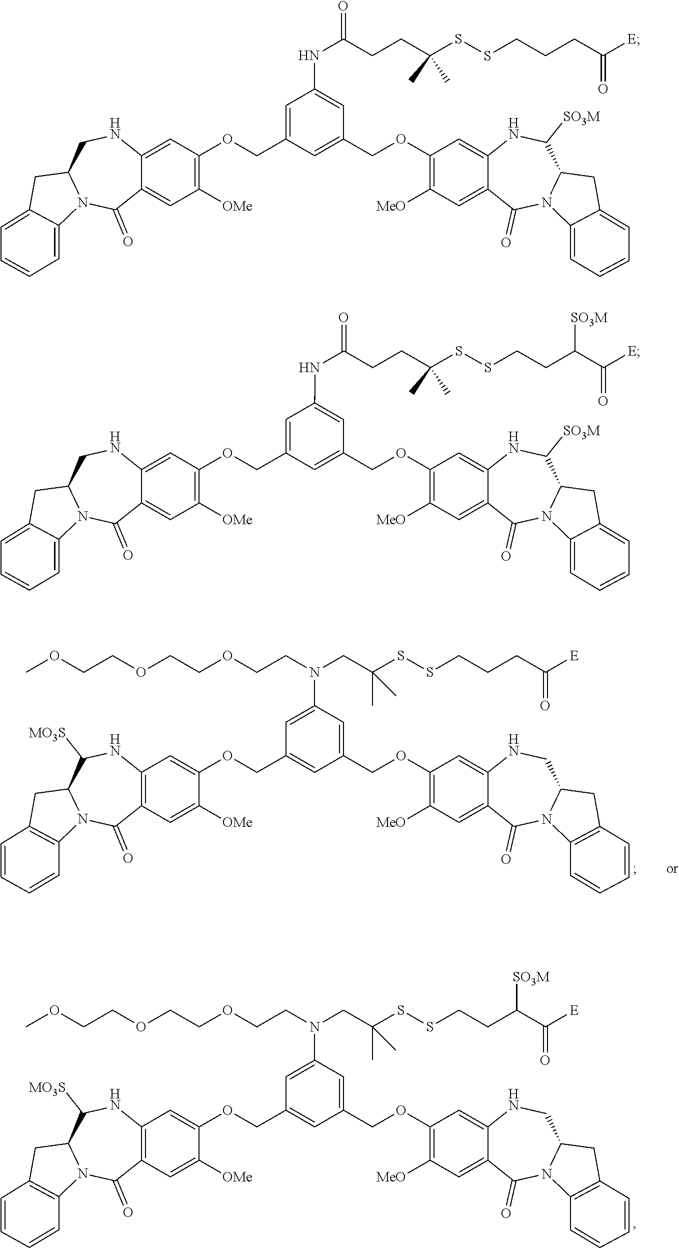
or a pharmaceutically acceptable salt thereof.

98. The method of claim 94, wherein the cytotoxic agent or the cytotoxic agent-linker compound is represented by one of the following formulas: 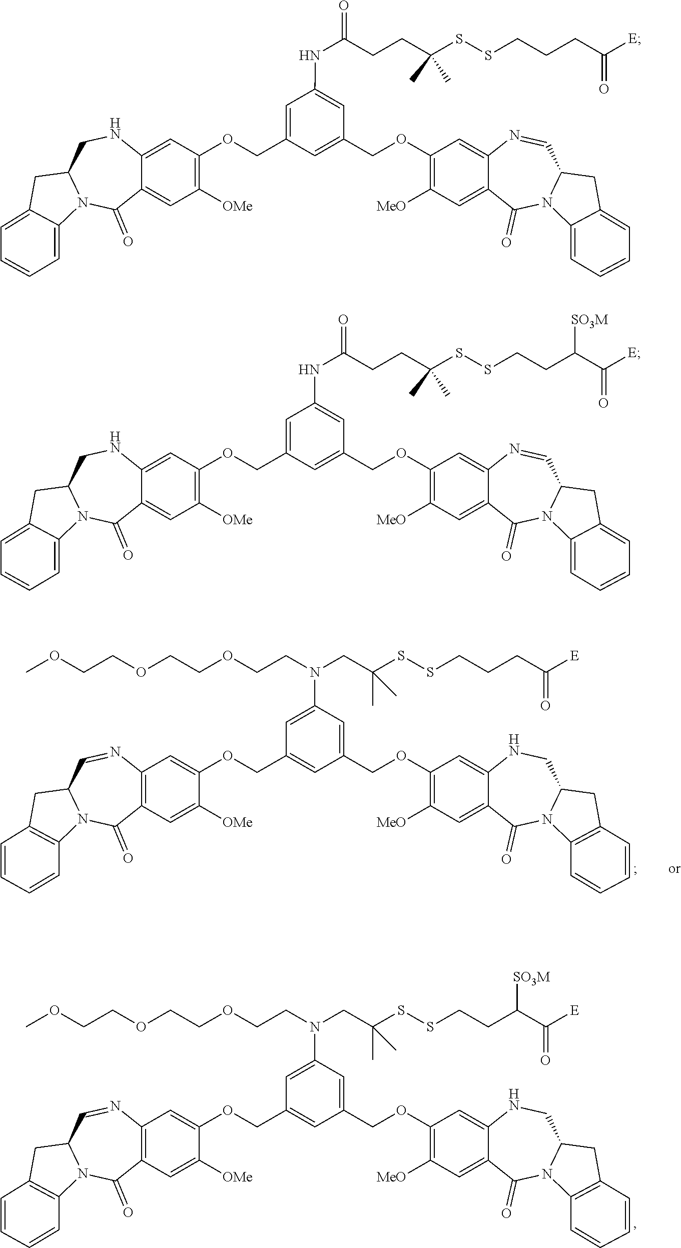
or a pharmaceutically acceptable salt thereof.

99. The method of any one of claims 94-98, wherein the reactive ester group is selected from N-hydroxysuccinimde ester, N-hydroxy sulfosuccinimide ester, nitrophenyl (e.g., 2 or 4-nitrophenyl) ester, dinitrophenyl (e.g., 2,4-dinitrophenyl) ester, sulfo-tetraflurophenyl (e.g., 4-sulfo-2,3,5,6-tetrafluorophenyl) ester, and pentafluorophenyl ester.

100. The method of claim 99, wherein the reactive ester group is represented by the following formula: 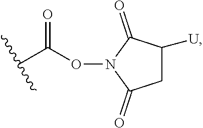
wherein U is H or —SO₃M.

101. The method of claim 100, wherein the reactive ester group is represented by the following formula:

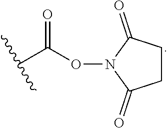 [SMILES: *C(=O)ON1C(=O)CCC1=O]

102. The method of claim 94, wherein the cytotoxic agent or the cytotoxic agent-linker compound is represented by one of the following structural formulas: 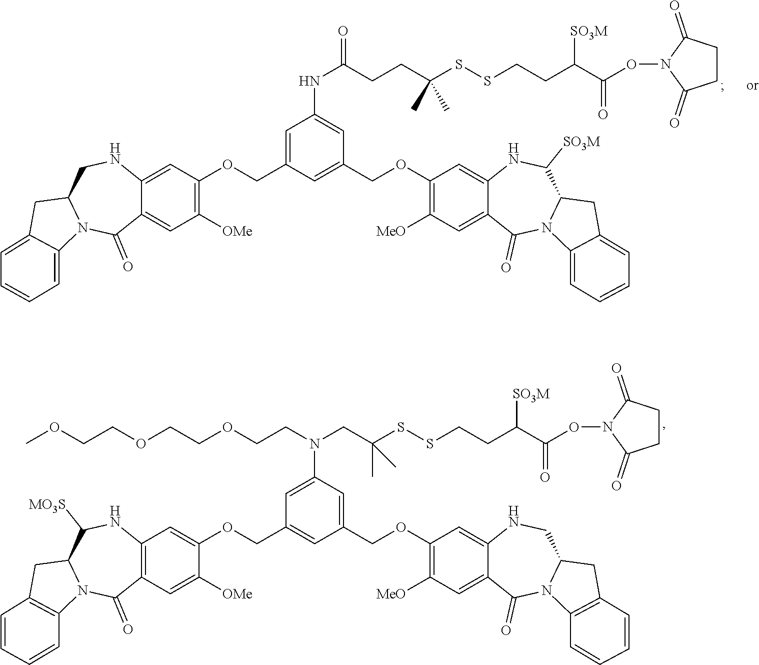
or a pharmaceutically acceptable salt thereof.

103. The method of claim 94, wherein the cytotoxic agent or cytotoxic agent-linker compound is represented by one of the following structural formulas: 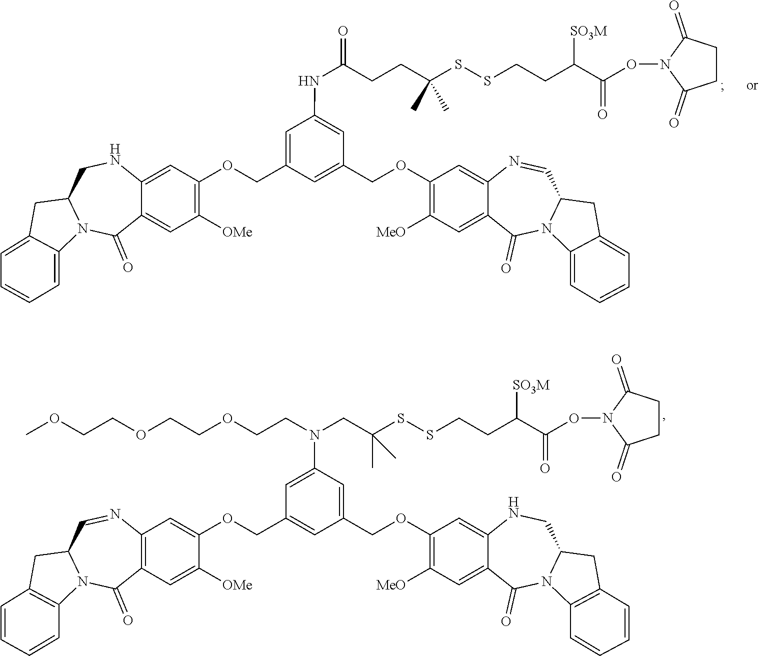
or a pharmaceutically acceptable salt thereof.

104. The method of any one of claims 94-96 and 99-101, wherein the cytotoxic agent or the cytotoxic agent-linker compound is represented by structural formula (III) prepared by reacting the compound of structural formula (IV) with a sulfonating reagent.

105. The method of any one of claims 94-96 and 99-101, wherein the cytotoxic agent or the cytotoxic agent-linker compound is represented by structural formula (V) prepared by reacting the compound of structural formula (VI) with a sulfonating reagent.

106. The method of any one of claims 97 and 99-101, wherein the cytotoxic agent or the cytotoxic agent-linker compound is prepared by reacting a compound represented by one of the following structural formulas: 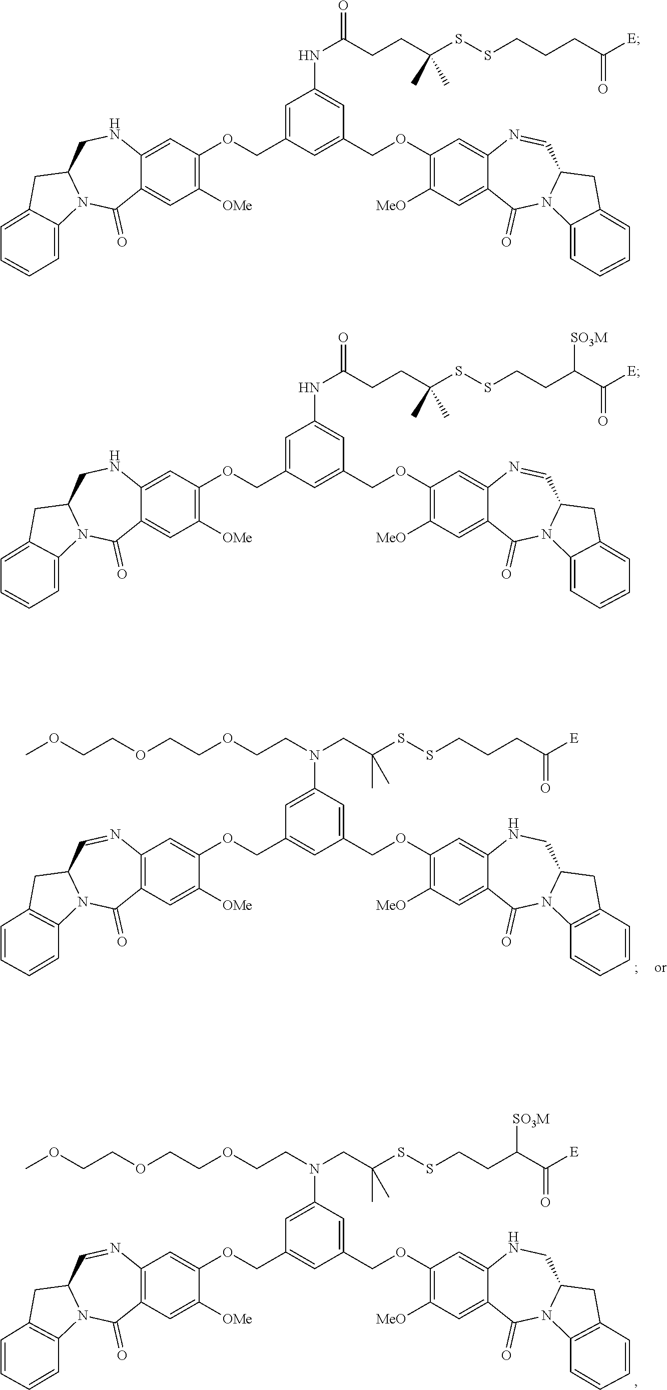
or a pharmaceutically acceptable salt thereof, with a sulfonating reagent.

107. The method of any one of claims 97 and 99-101, wherein the cytotoxic agent-linker compound is prepared by reacting a cytotoxic agent represented by one of the following structural formulas: 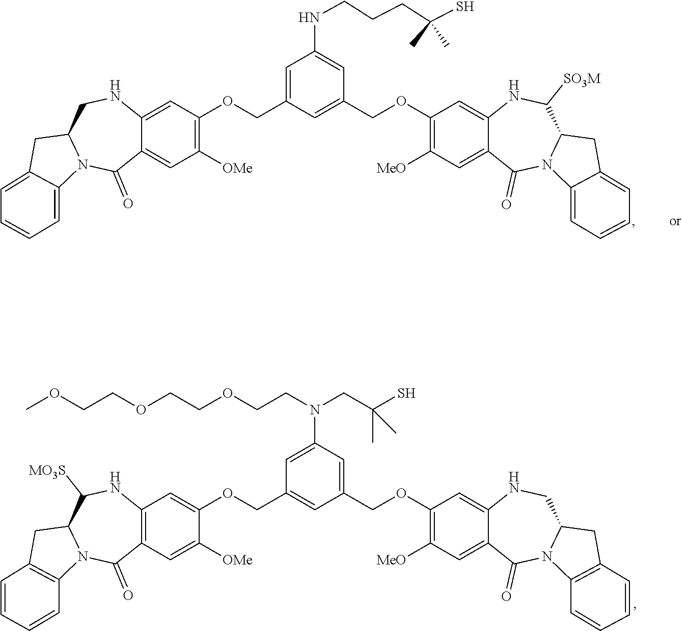
or a pharmaceutically acceptable salt thereof, with a linker compound represented by one of the following structural formulas:

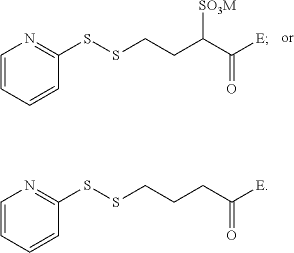

108. The method of claim 102, wherein the cytotoxic agent-linker compound is prepared by reacting a compound with one of the following structural formulas: 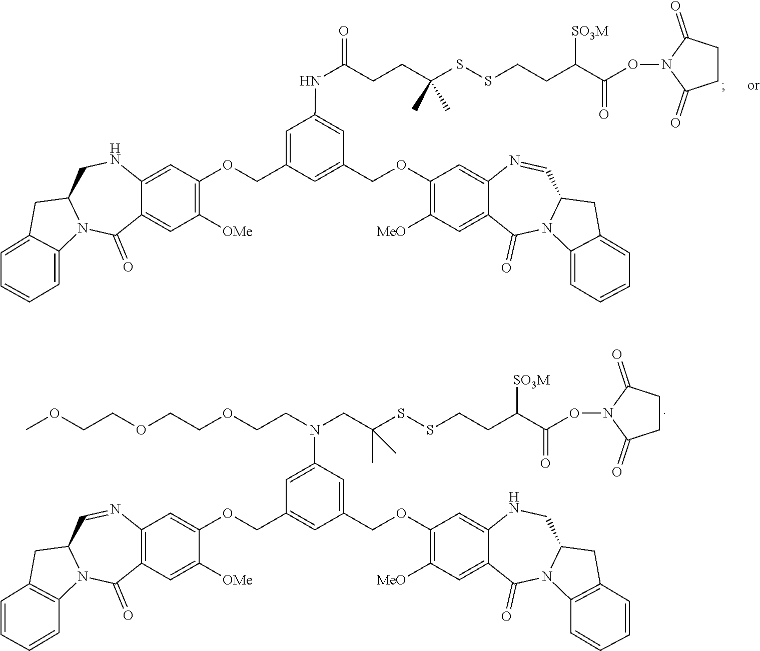
or a pharmaceutically acceptable salt thereof, with a sulfonating reagent.

109. The method of claim 102, wherein the cytotoxic agent-linker compound is prepared by reacting a cytotoxic agent represented by the following structural formula: 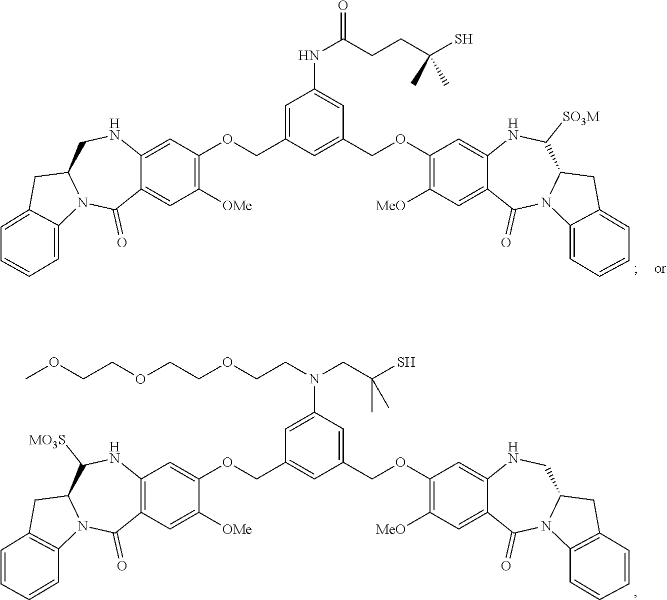
or a pharmaceutically acceptable salt thereof, with a linker compound of the following structural formula:

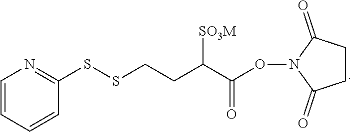

110. The method of any one of claims 94-96 and 99-101, wherein the cytotoxic agent or the cytotoxic agent-linker compound is represented by structural formula (IV) or (VI), and wherein the method further comprises reacting the cell-binding agent-cytotoxic agent conjugate with a sulfonating reagent.

111. The method of any of one of claims 94-96 and 99-101, wherein the cytotoxic agent or cytotoxic agent-linker compound is represented by structural formula (IV) or (VI), and wherein the method comprises reacting the compound represented by structural formula (IV) or (VI) in the presence of a sulfonating reagent.

112. The method of any one of claims 98-101 and 103, wherein the method further comprises reacting the cell-binding agent-cytotoxic agent conjugate with a sulfonating reagent.

113. The method of any one of claims 98-101 and 103, wherein the method comprises reacting the cell-binding agent with the cytotoxic agent or cytotoxic agent-linker compound in the presence of a sulfonating reagent.

114. The method of any one of claims 87-93 and 104-113, wherein the sulfonating reagent is NaHSO₃.

115. The method of any one of claims 72-113, wherein M is —H, Na⁺ or K⁺.

116. The method of claim 115, wherein M is Na⁺.

117. The method of any one of claims 1-116, wherein the cell-binding agent is an antibody.

118. The method of claim 117, wherein the antibody is a monoclonal antibody.

119. The method of claim 118, wherein the antibody is a humanized monoclonal antibody.


SEQUENCE LISTING (24 sequences)

SEQ ID NO: 1 - protein, 5 aa, artificial
  FOLR1 antibody heavy chain CDR1
GYFMN

SEQ ID NO: 2 - protein, 17 aa, artificial
  FOLR1 antibody heavy chain CDR2
  X (14)..(14): Lys, Gln, His, or Arg
  X (16)..(16): Gln, His, Asn, or Arg
  X (17)..(17): Gly, GLu, Thr, Ser, Ala, or Val
RIHPYDGDTFYNQXFXX

SEQ ID NO: 3 - protein, 9 aa, artificial
  FOLR1 antibody heavy chain CDR3
YDGSRAMDY

SEQ ID NO: 4 - protein, 15 aa, artificial
  FOLR1 antibody light chain CDR1
KASQSVSFAGTSLMH

SEQ ID NO: 5 - protein, 7 aa, artificial
  FOLR1 antibody light chain CDR2
RASNLEA

SEQ ID NO: 6 - protein, 9 aa, artificial
  FOLR1 antibody light chain CDR3
QQSREYPYT

SEQ ID NO: 7 - protein, 17 aa, artificial
  FOLR1 antibody heavy chain CDR2
RIHPYDGDTFYNQKFQG

SEQ ID NO: 8 - protein, 448 aa, artificial
  FOLR1 antibody heavy chain
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 112 aa, artificial
  FOLR1 antibody light chain variable domain
DIVLTQSPLSLAVSLGQPAIISCKASQSVSFAGTSLMHWYHQKPGQQPRLLIYRASNLEAGVPDRFSGSGSKTDFTLNISPVEAEDAATYYCQQSREYPYTFGGGTKLEIKR

SEQ ID NO: 10 - protein, 112 aa, artificial
  FOLR1 antibody light chain variable domain
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKR

SEQ ID NO: 11 - protein, 5 aa, artificial
  anti-GCC antibody heavy chain CDR1
GYYWS

SEQ ID NO: 12 - protein, 16 aa, artificial
  anti-GCC antibody heavy chain CDR2
EINHRGNTNDNPSLKS

SEQ ID NO: 13 - protein, 11 aa, artificial
  anti-GCC antibody heavy chain CDR3
ERGYTYGNFDH

SEQ ID NO: 14 - protein, 11 aa, artificial
  anti-GCC antibody light chain CDR1
RASQSVSRNLA

SEQ ID NO: 15 - protein, 7 aa, artificial
  anti-GCC antibody light chain CDR2
GASTRAT

SEQ ID NO: 16 - protein, 9 aa, artificial
  anti-GCC antibody light chain CDR3
QQYKTWPRT

SEQ ID NO: 17 - protein, 119 aa, artificial
  anti-GCC antibody heavy chain variable domain
QVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 18 - protein, 107 aa, artificial
  anti-GCC antibody light chain variable domain
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIK

SEQ ID NO: 19 - protein, 468 aa, artificial
  anti-GCC antibody heavy chain
MGWSCIILFLVATATGVHSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 233 aa, artificial
  anti-GCC antibody light chain
MGWSCIILFLVATATGVHSEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 4 aa, artificial
  Synthetic peptide
ALAL

SEQ ID NO: 22 - protein, 4 aa, artificial
  Synthetic peptide
  X (1)..(1): beta-Ala
XLAL

SEQ ID NO: 23 - protein, 4 aa, artificial
  Synthetic peptide
GFLG

SEQ ID NO: 24 - protein, 118 aa, artificial
  FOLR1 antibody heavy chain variable domain
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS